SEQUENCE LISTING (6 sequences)

SEQ ID NO: 1 - DNA, 762 bp, Homo sapiens
  source 1..762: mol_type = genomic DNA
cgtggttgctgcgtgtgtcccgcagccccctgcagccagcgtctcctcggccccggcccgaccctgccgtccctgctctgctccctcagcgaccgcagacccctcacgcacatgcccagccctgcagtcctactctgccaccccaaggggcttgggcttcagtgggggcagcgtgcggggcgtggagagggagacgtgtaaagccctgagaggctctgcagccacagtcccgcagtcagcgttccctctcggccaagcctcctggctgcacatcgtttcccgggcatctcagtgagaccggcctcggctcacagcggcacgttgtttttacacttcggggactctggtgggaccgcgggaagccaggaggcgggcgcgccccgggagccgataggaaagtgcaacagcgccatctagtggccgcgcggggaggctgcggagcgcgcgccgcgacccgggatccaccgtccgcaggaaaacgtgttccctccgatgcgtggtcacagcgcggcagcgcggccttgggagcttcttaaacaacaggttagctatcgctgccgcctcagtaagagaagtcaaggccaaaacgcttaaaggattttgacctattcggcttggtggagagcgctaaaaggggacgctatagcgggattctcagctctcctgggctagtgggacccccggtgcgccgccgccgatggggtcttagggcctgtctgtctgggctagagccggcccgggagcctgtttgcggggagtgcg

SEQ ID NO: 2 - DNA, 791 bp, Homo sapiens
  source 1..791: mol_type = genomic DNA
atctccgatactcctctcctcagcttcccgggggcagtatgtccctttgggtcatggcctgtggagtggccatgacccaaagttcaattaaggctggcgggtccccagaggggcctgcttccattcccccttccccgcagccctgcaggcgtcagcatggggcgaggttaccagttcctgacccagcagctcgtcctcaggtggtcctgctgctcgggaggtcagctgcgtggggagcctgtccacctggctgatgggggtgatggccgcgtggctgggattcctcctccagccctgcccttcaccgccatacagtctgggcagccccttcctgtcccgtcctcctgcctcgccctctgctggggtctacggggcccaagtcttgctggaacacaggcaggacaagggttctggaggttcaggccggaagtgaaataaaagtcgagaggggccgggcccggcacccaccgcatcctgcctccgggcctcctcgggcccctcacacggtggggcagcccctcctccagtggagcggaacggtgggccccgctgcccctccacccctgcgtgtgacacggacgtgcttggaagctgggttcgtcctttcagttttgtttcctggttggaaacccctgagactcctccccctccccccgcccctgcccacctgtgctttcccaggctggaaggggcagcccctctcagcacgtgggtggttaggctgtgacgtccccaggcctcctgggcctgcaccggggtccacccaggtgtgggccatgcccaggtgcggg

SEQ ID NO: 3 - DNA, 625 bp, Homo sapiens
  source 1..625: mol_type = genomic DNA
cggtttcgctccctatcggggccagggacgcctcaggctgtctcggtcaggacctacagctccggttgttgtcccaggctcttccgcgaggtgctctcctgtctcctgaccacccccattcctccccactccagctcctcagcgagcggctgcgaaggacgcgcacaacaacactccgcgcagcgggaaggtaccgaactcgcatcgcagcctgaaactgctcaactaagctcccgccccgctgctctctggtcaatctaaagcgaagacgagccttagggccaatcagaagcgacagcggtggagtcatgcccgcctgcttgaggcgccctggcgtctcattggctatgcttgagaacgaatcccaggctaagccacttagaaaggagcggagccagccaatcagcggtgcaaccgccgcgggggccgggccagaagccccgcagacaagcacctcgggagactggcgaggggcgagctcgcagcttgttagccccgaagccctgcccagggggaaaccctgctggagggagtctaacccccgggccagttaatgttggggcagcgcaatcggccgttccctctgcggtatggttggaggtgggagtgctgacacgtccgcgcg

SEQ ID NO: 4 - DNA, 658 bp, Homo sapiens
  source 1..658: mol_type = genomic DNA
gcctggggccactgctcctgggtcctcaggactgcctgggggaaggtagtgcattgtgcagcgcgcggtccagaagtgaaaagggaggcgcggagataagctgccggcggaagttccctctcctgcctgggccgacccggcgctttactgcttctcacgaaggtgcgccggctgctccagaaatcgcagactgcctccaggaagaacttgctggagtcacagcagcttctcagcgacttgacagcagtgattcagacttcaacttgggcggaggggcgggggaggagaaagagatttccagagaaaacgactgagcggtagggaggggaagagagaccgagccacgcgcctcgagaagcagtgcagagagcggaagagacagaggctgcgagacctacccacagagaccaagagagacgctcggaggggagaccgccttaggcgcagagattcagaggcagacagacaggcagacagaaggatacaggaaaggaatgtcgccgaaaggcagggacaaacctgagtcccagaaaaataagagacaactcccacacaccaggctgtccgcgggccgccttgtcacagaaaggcagctccccagccccgcagagtcccgacagctgcccccgcgaaggtggggcgaggggcggcttttccg

SEQ ID NO: 5 - DNA, 1355 bp, Homo sapiens
  source 1..1355: mol_type = genomic DNA
cggaccaaatataaccatttctaaaagcattcctcagcgcttggaacttgcgagctgatggcttttggtggagcccaggaggggcgctgtggggcacagacgtgtgcggggccggcagctgcggagagagcagcgcaccccagccttcaattccccgccgaagatgggactctgcccagagccgaggcccctggctgctgccccgctcccttgcggccgcagtgacactcagagccagattcctcagacaatcttcactcctccgtggtggctgccttgggaggcgttgcggggtgaaggaagtcgcagctggtcccaagcagtggggagggcaggacagggagtaagacaccgccttcctggggtgcacacggaggaggggcgctgcccaggagcggaggggaggggagcggagcgcggagaggagaccggtgccagacttcacccgcctagctctcggaacccccgggggccgcgctcggctccgcctgggggctaccggttctgggccaggagaagaagtggcatccgcggggcagggcgggcggtgacggccggctcaggtggcggccccaccccagcgcccttcagcaggcctgggtcggtcggcctcggaccccgtaggcgccggcggagctggcggcagggacccgacagctctgccgcggactccgcaggggacatcccccaccctggcggcccacccaggcggagcgcggcggggaccgtgcatctagcgcgccctggtgacctgcgggccgaggcgggctgtggggcttggccccgcgactcagccttccgggcccacagcggccctgacccgggaagtgcggaggtttggccgccttcaacagcacctgccaggccactgggagcagcgggggagaagtgggctcgcacaccgacggcctcccgggttcggcggggacgacccgagctaggagcccgcgggggccgtgggagctgctccgccgaggtggacccggggcgcccgcacccctcaccttctttccgtggggtccggggccagggacgcagcgggggacggcgggcatcagccgggcagcgcccgagccgtccctggccgggtccccacacctgcgtgctcgggcatccccagggtcgggggcgcggtgaatcgtggtccccgcagcccggggcccgccaggcccgcggagtcagcccaccctggcgaggggcccaggcacccgcatttaaccagtcccgacctcggcacgtggcggcgccggtgtcgcagactcgggcccacttctgggagctttggtgcgagggggaaagtgcccctgcggacgggctcgggtgcccggtgctgccccgccgccatccccggcacg

SEQ ID NO: 6 - DNA, 545 bp, Homo sapiens
  source 1..545: mol_type = genomic DNA
cgcgcctcccggaaccacgcgtctctgtgcacagacattcctgggggcaggctcctgtcctttaacacagtctcaaaggagtcttcaaaaacaaaaagtttacaagcactgatagggaaaacagaaggatgataacaccaagagggactttcccgggagaggacggcttccagggactgagaaaggatgggcaagtgggcggggcccggcgccgtgcgggcagggctggcgccgggagtccccagactcccccgcagtgggaagcacctctcccattcacgccgggcaggacacctggccgggcgggggaggcagcgcaagggccggccggggagtacgggactcgagccggggacctgaggcaggagccaagcatcgtcgcagggcaaccagcagaacggagagggaggcgcgggggcgaaggctggcgggagccgcgctgagggcaggagcccggagccccctagggcagcgccgatccgcccgccccgtcccgccgagctgggcctccgtctgtggcctgcgcagccagggtcgccaagccg

(19) United States
(12) Patent Application Publication
(10) Pub. No.: US 2026/0209856 A1
(43) Pub. Date: Jul. 23, 2026

(54) COMPOSITIONS AND METHODS FOR DETECTING ESOPHAGEAL CANCER

(71) Applicant: Mayo Foundation for Medical Education and Research, Rochester, MN (US)

(72) Inventors: David A. Ahlquist, Rochester, MN (US); John B. Kisiel, Rochester, MN (US); Douglas W. Mahoney, Rochester, MN (US); Seth W. Slettedahl, Rochester, MN (US); Zhifu Sun, Rochester, MN (US); William R. Taylor, Rochester, MN (US)

(21) Appl. No.: 19/140,037

(22) PCT Filed: Dec. 20, 2023
(86) PCT No.: PCT/US23/85117

§ 371 Date: Jun. 17, 2025

Related U.S. Application Data
(60) Provisional application No. 63/433,784, filed on Dec. 20, 2022.

(51) Int. Cl. C12Q 1/6886; C12Q 1/6806; C12Q 1/6809

(52) U.S. Cl. CPC C12Q 1/6886; C12Q 1/6806; C12Q 1/6809; C12Q 2600/154

(57) ABSTRACT

The present disclosure provides compositions and methods for distinguishing non-cancerous, pre-cancerous, and cancerous conditions in the esophagus. In particular, the present disclosure provides compositions and methods for distinguishing non-dysplastic Barrett's Esophagus (NDBE) samples from precancerous (e.g., low or high grade dysplasia) and/or cancerous (e.g., esophageal adenocarcinoma) samples based on methylation status and/or DNA copy number aberrations.

### CROSS REFERENCE TO RELATED APPLICATIONS

This application claims priority to and the benefit of U.S. Provisional Patent Application No. 63/433,784 filed Dec. 20, 2022, which is incorporated herein by reference in its entirety and for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 10,938 Byte file named “41473-601_SEQUENCE_LISTING” created on Dec. 14, 2023.

### FIELD

The present disclosure provides compositions and methods for distinguishing non-cancerous, pre-cancerous, and cancerous conditions in the esophagus. In particular, the present disclosure provides compositions and methods for distinguishing non-dysplastic Barrett's Esophagus (NDBE) and/or normal esophageal samples from precancerous (e.g., low or high grade dysplasia) and/or cancerous (e.g., esophageal adenocarcinoma) samples based on methylation status and/or DNA copy number aberrations.

### BACKGROUND

Barrett's Esophagus (BE) is a condition in which tissue that is similar to the tissue lining in the intestines changes or replaces the lining of the esophagus. Even though BE has a chance of progressing into low-grade dysplasia (LGD) or high-grade dysplasia (HGD), which are precancerous conditions that can transform into esophageal adenocarcinoma (EAC), in most cases, the dysplasia may not be presented or cannot be identified by biopsy specimens. This condition is termed non-dysplastic BE (NDBE). Generally, endoscopic surveillance is recommended to identify and differentiate dysplastic, pre-cancerous, and cancerous conditions. However, this diagnostic paradigm is limited by esophageal sampling error and subtle histologic changes, leading to delayed treatment of dysplasia and EAC.

### SUMMARY

Embodiments of the present disclosure provide methods, compositions, and systems for screening various types of esophageal disorders in a biological sample. In accordance with these embodiments, the present disclosure includes, but is not limited to, methods and compositions for detecting the presence of esophageal cancer or pre-cancer from a biological sample. In some embodiments, the biological sample is a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample. In some embodiments, the tissue sample is an esophageal tissue sample. In some embodiments, the esophageal sample is obtained from an esophageal biopsy, or by swabbing, brushing, or using a sponge capsule device.

As described further herein, embodiments of the present disclosure include novel differentially methylated regions (DMRs), each individually capable of distinguishing esophageal cancer or pre-cancer from control or benign tissue. In some embodiments, the novel DMRs are capable of distinguishing high grade dysplastic Barrett's Esophagus (HGD-BE) or esophageal adenocarcinoma (EAC) from non-dysplastic Barrett's Esophagus (NDBE) or a normal esophageal control. In accordance with these embodiments, the novel DMR(s) is from a gene selected from ACVRL1, ADAMTS8, ADAP2, ADRBK2, AKR1B1, ANK1, ANKRD13B, ANXA6, ARNT2, B4GALNT2, BACH2, BCL11A, BSCL2, C12orf53, C14orf82, C17orf107, C18orf1, C1orf95, C5orf42, CACNA1C, CAMK1D, CAMTA1, CBX6, CCDC85A, CCKBR, CD38, CDKN2A, CH25H, CHST1, CHST15, CNTLN, CRHR1, CRTC1, CXCR4, CYP1B1, DCTN2, DIDO1, DMKN, DSE, DYNC1I1, EML6, ENOX1, EPHA4, ESRRG, FAM176A, FAM78B, FBXO10, FERMT2, FHOD3, FLJ45079, FMNL1, FOXP2, FRMD4B, FZD8, GALNTL1, GALNTL4, GAS1, GBGT1, GLIPR2, GNAI1, GNAL, GPR37, GRASP, GRID1, GRM8, GSC, HAR1A, HCN2, HEY2, HIST1H2BE, HS3ST3B1, HTR7, IGFBP2, INHA, INSRR, IRX3, ISM2, KCNG3, KCNK4, KCNS2, KCTD15, KIAA1522, KIAA1614, KIF26A, KL, KLF15, KLHL10, KRT77, KSR2, LBH, LMX1A, LMX1B, LOC100526820, LONRF2, LRFN2, LRRN1, MAF, MAFB, MARK1, ADAMTSL4-AS1, PGBD5, HSPA12A, SFTPD, LOC107984507, LOC100128253, CISTR, SLC16A7, CTXND1, MAX.chr15.4912, ZNF423, RBFOX1, LOC105376772, GSE1, MAX.chr17.8070, ZNF709, MAX.chr19.3439, GREB1, CYP27C1, AC068134.6, LOC388780, STOX2, PPARGC1A, MAX.chr4.4552, PDZD2, MAX.chr6.6743, LPAL2, CDK14, MAX.chr8.3003, MCOLN2, MEGF11, MFSD11, MRC2, MSX1, NAT8L, NAV1, NBEA, NCRNA00092, NECAB2, NEURL, NGEF, NHLH2, NKD1, NLGN1, NOG, NPPC, NR3C1, NRXN2, NTN1, OBSCN, OCA2, OSBPL1A, OXR1, P2RY1, PDE2A, PDE9A, PDGFRA, PID1, PLCG2, PLCL1, PNPLA3, POU3F1, POU3F2, PRKACB, PRKAR2B, PRKG1, PRR18, PRR5L, PTHLH, PYGL, RARG, RHBDL3, RIMS2, ROR2, RPRML, SDK2, SOX9, SYNGR1, TAC4, TNFRSF19, TRANK1, TSPAN33, TSPAN4, TSPAN5, UBE2E2, UCHL1, UNC5A, VASH2, VIM, WNT6, ZBTB10, ZNF680, ZNF738, ZNF808, and ZNF845 (Table 1), including any combinations thereof. In some embodiments, the novel DMR(s) is from any gene selected from Table 1, including any combinations thereof. Each novel DMR alone is capable of distinguishing HGD-BE and/or EAC from NDBE and/or a control sample, and combining two or more of the novel DMRs can provide increased sensitivity. Therefore, combinations of two or more novel DMRs selected from Table 1 are provided.

Embodiments of the present disclosure also include novel differentially methylated regions (DMRs), each individually capable of distinguishing esophageal cancer or pre-cancer from control or benign tissue. In some embodiments, the novel DMRs are capable of distinguishing high grade dysplastic Barrett's Esophagus (HGD-BE) or esophageal adenocarcinoma (EAC) from non-dysplastic Barrett's Esophagus (NDBE) or a normal esophageal control. In accordance with these embodiments, the novel DMR(s) is from a gene selected ACVRL1, ADAMTS8, ADAP2, ADRBK2, AKR1B1, ANK1, ANKRD13B, ANXA6, ARNT2, B4GALNT2, BACH2, BCL11A, BSCL2, C14orf82, C18orf1, C1orf95, C5orf42, CAMK1D, CAMTA1, CCDC85A, CD38, CDKN2A, CHST1, CHST15, CRHR1, CYP1B1, DIDO1, DSE, DYNC1I1, EML6, ENOX1, EPHA4, FAM176A, FBXO10, FERMT2, FHOD3, FLJ45079, FMNL1, FRMD4B, FZD8, GALNTL1, GALNTL4, GAS1, GBGT1, GLIPR2, GNAI1, GNAL, GRASP, GRID1, GRM8, GSC, HAR1A, HCN2, HEY2, HIST1H2BE, HS3ST3B1, HTR7, IGFBP2, INHA, IRX3, ISM2, KCNG3, KCNK4, KCNS2, KCTD15, KIAA1614, KL, KLHL10, KSR2, LBH, LMX1A, LMX1B, LOC100526820, LONRF2, LRFN2, MAFB, MARK1, PGBD5, HSPA12A, SFTPD, LOC107984507, LOC100128253, CISTR, SLC16A7, CTXND1, ZNF423, LOC105376772, GSE1, MAX.chr19.3439, GREB1, CYP27C1, AC068134.6, LOC388780, STOX2, PPARGC1A, PDZD2, MAX.chr6.6743, LPAL2, CDK14, MCOLN2, MEGF11, MRC2, MSX1, NAT8L, NAV1, NBEA, NCRNA00092, NEURL, NGEF, NHLH2, NKD1, NLGN1, NOG, NPPC, NR3C1, NRXN2, OBSCN, OCA2, OSBPL1A, OXR1, P2RY1, PDE2A, PDGFRA, PID1, PLCG2, PLCL1, PNPLA3, POU3F1, POU3F2, PRKACB, PRKAR2B, PRKG1, PRR18, PRR5L, PTHLH, PYGL, RHBDL3, RIMS2, ROR2, RPRML, SDK2, SOX9, SYNGR1, TAC4, TRANK1, TSPAN4, UBE2E2, UCHL1, UNC5A, VASH2, ZBTB10, ZNF680, ZNF709, ZNF738, ZNF808, and ZNF845 (Table 2), including any combinations thereof. In some embodiments, the novel DMR(s) is from any gene selected from Table 2, including any combinations thereof. Each novel DMR alone is capable of distinguishing HGD-BE and/or EAC from NDBE and/or a control sample, and combining two or more of the novel DMRs can provide increased sensitivity. Therefore, combinations of two or more novel DMRs selected from Table 2 are provided.

Embodiments of the present disclosure also include novel differentially methylated regions (DMRs), each individually capable of distinguishing esophageal cancer or pre-cancer from control or benign tissue. In some embodiments, the novel DMRs are capable of distinguishing high grade dysplastic Barrett's Esophagus (HGD-BE) or esophageal adenocarcinoma (EAC) from non-dysplastic Barrett's Esophagus (NDBE) or a normal esophageal control. In accordance with these embodiments, the novel DMR(s) is from a gene selected from BACH2, C5orf42, FHOD3, HIST1H2BE, IRX3, KIAA1614, LONRF2, MAFB, PDGFRA, PID1, POU3F1, PRR5L, RHBDL3, and SDK2 (Table 3), including any combinations thereof. In some embodiments, the novel DMR(s) is from any gene selected from Table 3, including any combinations thereof. Each novel DMR alone is capable of distinguishing HGD-BE and/or EAC from NDBE and/or a control sample, and combining two or more of the novel DMRs can provide increased sensitivity. Therefore, combinations of two or more novel DMRs selected from Table 3 are provided.

Embodiments of the present disclosure also include novel differentially methylated regions (DMRs), each individually capable of distinguishing esophageal cancer or pre-cancer from control or benign tissue. In some embodiments, the novel DMRs are capable of distinguishing high grade dysplastic Barrett's Esophagus (HGD-BE) or esophageal adenocarcinoma (EAC) from non-dysplastic Barrett's Esophagus (NDBE) or a normal esophageal control. In accordance with these embodiments, the novel DMR(s) is from a gene selected from KL, PGBD5, ROR2, and LMX1B (Example 4), including any combinations thereof. In some embodiments, the novel DMR(s) is from any gene selected from Example 4, including any combinations thereof. Each novel DMR alone is capable of distinguishing HGD-BE and/or EAC from NDBE and/or a control sample, and combining two or more of the novel DMRs can provide increased sensitivity. Therefore, combinations of two or more novel DMRs selected from Example 4 are provided.

In some embodiments, the novel DMR(s) capable of distinguishing esophageal cancer or pre-cancer from control or benign tissue were validated using at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR, and based on at least one of an area under a ROC curve (AUC), fold-change in methylation, methylation percentage, and/or hypermethylation ratio between a test sample and a control sample.

In accordance with the above, a control sample comprises a sample from a subject that does not have cancer, a sample from a subject that does not have esophageal cancer, a sample from a subject that does not have esophageal pre-cancer, or a sample from a subject that has a type of cancer that is not an esophageal cancer or pre-cancer. In some embodiments, a control sample comprises a sample from a subject that has non-dysplastic Barrett's Esophagus (NDBE). In some embodiments, the control sample is from a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and a stool sample. In some embodiments, the tissue sample is an esophageal tissue sample. In some embodiments, the esophageal sample is obtained from an esophageal biopsy, or by swabbing, brushing, or using a sponge capsule device.

In some embodiments, the novel DMR(s) capable of distinguishing esophageal cancer or pre-cancer from a control sample is associated with an area under a ROC curve (AUC) greater than or equal to 0.5, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.6, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.7, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.75, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.8, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.85, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.9, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.95, wherein the ROC curve discriminates between a subject having or suspected of having esophageal cancer or pre-cancer and a control DNA sample.

In some embodiments, the novel DMR(s) capable of distinguishing esophageal cancer or pre-cancer from a control sample comprises an increased methylation percentage as compared to a control DNA sample. In some embodiments, the novel DMR(s) capable of distinguishing esophageal cancer or pre-cancer from a control sample a control sample comprises an increased hypermethylation ratio as compared to a control DNA sample.

Embodiments of the present disclosure also include methods and compositions for characterizing a biological sample and determining a methylation profile in at least one differentially methylated region (DMR) of a DNA sample obtained from a subject having or suspected of having an esophageal cancer or pre-cancer by treating the sample with a reagent that modifies DNA in a methylation-specific manner. In some embodiments, the method includes detecting the presence of esophageal cancer or pre-cancer from a biological sample. In some embodiments, the at least one DMR is capable of distinguishing high grade dysplastic Barrett's Esophagus (HGD-BE) or esophageal adenocarcinoma (EAC) from non-dysplastic Barrett's Esophagus (NDBE) or a normal esophageal control.

In accordance with these embodiments, the method includes determining copy number variation (CNV) for the DNA sample from the subject. In some embodiments, the CNV discriminates between a subject having or suspected of having high grade dysplastic Barrett's Esophagus or esophageal adenocarcinoma (EAC) and a control DNA sample. In some embodiments, the at least one DMR comprises an increased CNV compared to a control DNA sample.

In some embodiments, the method includes determining an aneuploidy score (AS) for the DNA sample from the subject. In some embodiments, the AS discriminates between a subject having or suspected of having high grade dysplastic Barrett's Esophagus or esophageal adenocarcinoma (EAC) and a control DNA sample. In some embodiments, the at least one DMR comprises an increased AS as compared to a control DNA sample.

As described further herein, the present disclosure provides the materials and methods for utilizing sequencing reads from a genome that has been modified in a methylation-specific manner, for instance a cytosine or 5-methylcytosine converted genome, to make copy number aberration (CNA) determinations, including polyploidy and aneuploidy (e.g., determine an aneuploidy score). Most currently available technologies use direct NGS on wild-type, unconverted DNA. However, embodiments of the present disclosure include the ability to perform methylation and CNV analysis from the same chemistry/dataset. In accordance with these embodiments, the methylation profile in the at least one DMR and the CNV and/or the AS can be determined using the same DNA sample obtained from a subject. In some embodiments, the methylation profile in the at least one DMR and the CNV and/or the AS can be determined using a single DNA sample obtained from the subject. In some embodiments, the sample (e.g., the same sample or the single sample) has been treated with a reagent that modifies DNA in a methylation-specific manner.

In some embodiments, the control DNA sample used in the method and compositions of the present disclosure is from a subject that does not have an esophageal cancer or pre-cancer. In some embodiments, the control DNA sample is from a subject that has non-dysplastic Barrett's Esophagus (NDBE). In some embodiments, the control DNA sample is selected from a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and a stool sample. In some embodiments, the tissue sample is an esophageal tissue sample or a gastric cardia sample.

In some embodiments, the biological sample is obtained from a human subject. In some embodiments, the biological sample is obtained from a human subject, and the method includes extracting the DNA sample from the biological sample. In some embodiments, the biological sample is collected with a collection device. For example, the biological sample can be an esophageal sample obtained from an esophageal biopsy, or by swabbing, brushing, or using a sponge capsule device.

In some embodiments, the methods of the present disclosure include using a reagent that modifies DNA in a methylation-specific manner. In some embodiments, the reagent is a borane reducing agent. In some embodiments, the reagent that modifies DNA in a methylation-specific manner comprises one or more of a methylation-sensitive restriction enzyme, a methylation-dependent restriction enzyme, and a bisulfite reagent.

In some embodiments, determining the methylation profile of at least one DMR comprises amplifying at least a portion of the DMR using a set of primers. In some embodiments, determining the methylation profile of at least one DMR comprises performing at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR. In some embodiments, determining the methylation profile of at least one DMR comprises determining the presence or absence of methylation at a CpG site. In some embodiments, the one or more CpG sites are present in a coding region, a non-coding region, and/or a regulatory region of a gene (e.g., any one of the genes disclosed herein).

Embodiments of the present disclosure also include a method of identifying an esophageal cancer or pre-cancer. In accordance with these embodiments, the method includes determining a methylation profile in at least one differentially methylated region (DMR) of a DNA sample obtained from a subject having or suspected of having an esophageal cancer or pre-cancer by treating the sample with a reagent that modifies DNA in a methylation-specific manner. In some embodiments, the methylation profile indicates that the subject has an esophageal cancer (e.g., esophageal adenocarcinoma (EAC)) or an esophageal pre-cancer (e.g., high grade dysplastic Barrett's Esophagus (HGD-BE)). In some embodiments, the method includes determining copy number variation (CNV) for the DNA sample from the subject. In some embodiments, the method includes determining an aneuploidy score (AS) for the DNA sample from the subject. In some embodiments, the method further includes treating the subject with an anti-cancer therapy.

### BRIEF DESCRIPTION OF THE DRAWINGS

FIG. 1: Representative bar graph of total CNV results at the level of chromosome arm gains and losses. (The higher the signal, the greater degree of chromosomal instability and aneuploidy.)

FIG. 2: Representative violin plot of total CNV results at the level of chromosome arm gains and losses. (The higher the signal, the greater degree of chromosomal instability and aneuploidy.)

FIG. 3: Representative chromosomal scatter plot illustrating normal euploidy (two copies of chromosomes/genes) for the normal esophagus (NE) cohort generated from whole genome sequencing data. Each point represents a binned genomic segment.

FIG. 4: Representative chromosomal scatter plot illustrating normal euploidy (two copies of chromosomes/genes) for the non-dysplastic Barrett's Esophagus (NDBE) cohort generated from whole genome sequencing data. Each point represents a binned genomic segment.

FIG. 5: Representative chromosomal scatter plot illustrating aneuploidy for the high grade dysplasia (HGD) cohort. These were generated from the whole genome NGS data. Each point represents a binned genomic segment.

FIG. 6: Representative chromosomal scatter plot illustrating aneuploidy for the esophageal adenocarcinoma (EAC) cohort. These were generated from the whole genome NGS data. Each point represents a binned genomic segment.

FIG. 7: Representative heatmap of the DMRs from the MAFB gene, the top methylation marker candidate identified in the present disclosure, for the high grade dysplasia (HGD) and esophageal adenocarcinoma (EAC) cohorts. The columns represent samples and the rows represent individual CpGs, in genomic order, which make up the DMR. Dark green indicates no methylation; increasing shades of red indicates increasing methylation intensity.

FIG. 8: Representative heatmap of the DMRs from the MAFB gene, the top methylation marker candidate identified in the present disclosure, for the normal esophagus (NE) and non-dysplastic Barrett's Esophagus (NDBE) cohorts. The columns represent samples and the rows represent individual CpGs, in genomic order, which make up the DMR. Dark green indicates no methylation; increasing shades of red indicates increasing methylation intensity.

FIG. 9: Representative heatmap illustrating complementarity between CNV (aneuploidy) and methylation analysis among the esophageal adenocarcinoma (EAC), high grade dysplasia (HGD), and non-dysplastic Barrett's Esophagus (NDBE) cohorts.

### DETAILED DESCRIPTION

Barrett's esophagus (BE) is the strongest risk factor for and only known precursor for esophageal adenocarcinoma (EAC), a lethal malignancy with poor survival (<20% at 5 years) when detected after the onset of symptoms. The incidence of esophageal adenocarcinoma has increased by almost 600% in the last three decades in the population. BE progresses to EAC through a step-wise pathway from no dysplasia (also referred to as non-dysplastic Barrett's Esophagus or NDBE), to low grade dysplasia (LGD), to high grade dysplasia (HGD), and then to carcinoma. This metaplasia to dysplasia to carcinoma sequence has prompted several national gastroenterology societies to recommend screening for BE in high risk subjects with multiple risk factors followed by endoscopic surveillance (depending on the grade of dysplasia) to detect the development of dysplasia or carcinoma at an early stage. Endoscopic treatments of LGD, HGD and early carcinoma have been developed and shown to be effective in reducing the incidence of carcinoma and improving survival in BE subjects.

Screening for BE is currently performed using conventional sedated endoscopy (sEGD), which reveals the replacement of the normal squamous lining of the esophagus by metaplastic columnar epithelium in subjects with BE. However sedated endoscopy is expensive with both direct and indirect costs and not suitable for widespread application. It is also associated with potential complications. Other techniques such as unsedated transnasal endoscopy (uTNE) have comparable accuracy to sEGD with lower cost, but continue to be poorly regarded as a widely applicable tool by providers. Despite adequate access to the uTNE device the utilization of uTNE by referring physicians remains limited. The absence of accurate risk stratification tools to determine BE risk and target screening efforts are additional limitations to a widely applicable BE screening.

Endoscopic detection of dysplasia is currently performed using four quadrant random biopsies every 1-2 cm of the BE segment in addition to careful inspection of the BE segment with high resolution white light imaging and advanced imaging techniques. While this has been recommended by GI societies, the compliance with these recommendations amongst practicing gastroenterologists remains poor. Indeed, compliance decreases with increasing BE segment length leading to increasing rates of missed dysplasia. Other challenges with dysplasia detection in BE include the spotty distribution of dysplasia in BE, which leads to sampling error, poor inter-observer agreement amongst pathologists while grading dysplasia and the relatively poor sensitivity of current surveillance strategies in detecting prevalent dysplasia or carcinoma. The utility of advanced imaging techniques in the community remains unclear with only a third of practicing gastroenterologists reporting use routinely in BE surveillance. Recently, a sponge on a string device has been studied in BE screening. This device consists of a polyurethane foam sponge compressed in a gelatin capsule, attached to a string. The capsule is swallowed by the patient. The gelatin shell of the capsule dissolves in the gastric fluid releasing the foam device as a sphere, which is then pulled out with the attached string, providing brushing/cytology samples of the proximal stomach and esophagus. Biomarker studies can then be performed on these samples to detect BE.

As described further herein, BE is a metaplastic change in the lining of the distal esophageal epithelium, characterized by replacement of the normal squamous epithelium (NE) by specialized intestinal metaplasia. The presence of Barrett esophagus increases the risk of esophageal adenocarcinoma several-fold. The present disclosure addresses the detection of high-grade dysplastic (HGD) Barrett's esophagus, including adenocarcinoma (EAC), distinct from non-dysplastic Barrett's esophagus (NDBE). Knowing the dysplastic status of a Barrett's patient is crucial for follow-up clinical surveillance and treatment strategies. As described further herein, RRBS analysis was performed on tissue biopsies and whole genome sequencing on endoscopic brushing samples to generate methylation and copy number variation profiles of BE patients. Using proprietary analysis algorithms, 199 methylation DNA markers (MDMs) were identified, 156 of which were confirmed in both NGS datasets. In addition, aneuploidy scores were developed using the WGS data, and these scores demonstrated how this marker class complements the MDM analysis. For the aneuploidy/CNV analysis, the converted genome from the methylation analysis was used to make the calls, which is an approach that is not currently used. Thus, this technology can be implemented in a clinical testing format for use with endoscopic brushings and non-endoscopic non-invasive esophageal sponge samples.

Section headings as used in this section and the entire disclosure herein are merely for organizational purposes and are not intended to be limiting.

### 1. DEFINITIONS

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

The term “one or more,” as used herein, refers to a number higher than one. For example, the term “one or more” encompasses any of the following: two or more, three or more, four or more, five or more, six or more, seven or more, eight or more, nine or more, ten or more, twelve or more, thirteen or more, fourteen or more, fifteen or more, twenty or more, fifty or more, 100 or more, or an even greater number.

The term “one or more but less than a higher number,” “two or more but less than a higher number,” “three or more but less than a higher number,” “four or more but less than a higher number,” “five or more but less than a higher number,” “six or more but less than a higher number,” “seven or more but less than a higher number,” “eight or more but less than a higher number,” “nine or more but less than a higher number,” “ten or more but less than a higher number,” “eleven or more but less than a higher number,” “twelve or more but less than a higher number,” “thirteen or more but less than a higher number,” “fourteen or more but less than a higher number,” or “fifteen or more but less than a higher number” is not limited to a higher number. For example, the higher number can be 10,000, 1,000, 100, 50, etc. For example, the higher number can be approximately 50 (e.g., 50, 49, 48, 47, 46, 45, 44, 43, 42, 41, 40, 39, 38, 37, 36, 35, 34, 33, 32, 31, 32, 30, 29, 28, 27, 26, 25, 24, 23, 22, 21, 20, 19, 18, 17, 16, 15, 14, 13, 12, 11, 10, 9, 8, 7, 6, 5, 4, 3 or 2).

The term “one or more methylated markers” or “one or more DMRs” or “one or more genes” or “one or more markers” or “a plurality of methylated markers” or “a plurality of markers” or “a plurality of genes” or “a plurality of DMRs” is similarly not limited to a particular numerical combination. Indeed, any numerical combination of methylated markers is contemplated (e.g., 1-2 methylated markers, 1-3, 1-4, 1-5. 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38) (e.g., 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38) (e.g., 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38) (e.g., 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 4-25, 4-26, 4-27, 4-28, 4-29, 4-30, 4-31, 4-32, 4-33, 4-34, 4-35, 4-36, 4-37, 4-38) (e.g., 5-6, 5-7, 5-8, 5-9, 5-10, 5-11, 5-12, 5-13, 5-14, 5-15, 5-16, 5-17, 5-18, 5-19, 5-20, 5-21, 5-22, 5-23, 5-24, 5-25, 5-26, 5-27, 5-28, 5-29, 5-30, 5-31, 5-32, 5-33, 5-34, 5-35, 5-36, 5-37, 5-38) (e.g., 6-7, 6-8, 6-9, 6-10, 6-11, 6-12, 6-13, 6-14, 6-15, 6-16, 6-17, 6-18, 6-19, 6-20, 6-21, 6-22, 6-23, 6-24, 6-25, 6-26, 6-27, 6-28, 6-29, 6-30, 6-31, 6-32, 6-33, 6-34, 6-35, 6-36, 6-37, 6-38) (e.g., 7-8, 7-9, 7-10, 7-11, 7-12, 7-13, 7-14, 7-15, 7-16, 7-17, 7-18, 7-19, 7-20, 7-21, 7-22, 7-23, 7-24, 7-25, 7-26, 7-27, 7-28, 7-29, 7-30, 7-31, 7-32, 7-33, 7-34, 7-35, 7-36, 7-37, 7-38) (e.g., 8-9, 8-10, 8-11, 8-12, 8-13, 8-14, 8-15, 8-16, 8-17, 8-18, 8-19, 8-20, 8-21, 8-22, 8-23, 8-24, 8-25, 8-26, 8-27, 8-28, 8-29, 8-30, 8-31, 8-32, 8-33, 8-34, 8-35, 8-36, 8-37, 8-38) (e.g., 9-10, 9-11, 9-12, 9-13, 9-14, 9-15, 9-16, 9-17, 9-18, 9-19, 9-20, 9-21, 9-22, 9-23, 9-24, 9-25, 9-26, 9-27, 9-28, 9-29, 9-30, 9-31, 9-32, 9-33, 9-34, 9-35, 9-36, 9-37, 9-38) (e.g., 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 10-31, 10-32, 10-33, 10-34, 10-35, 10-36, 10-37, 10-38) (e.g., 11-12, 11-13, 11-14, 11-15, 11-16, 11-17, 11-18, 11-19, 11-20, 11-21, 11-22, 11-23, 11-24, 11-25, 11-26, 11-27, 11-28, 11-29, 11-30, 11-31, 11-32, 11-33, 11-34, 11-35, 11-36, 11-37, 11-38) (e.g., 12-13, 12-14, 12-15, 12-16, 12-17, 12-18, 12-19, 12-20, 12-21, 12-22, 12-23, 12-24, 12-25, 12-26, 12-27, 12-28, 12-29, 12-30, 12-31, 12-32, 12-33, 12-34, 12-35, 12-36, 12-37, 12-38) (e.g., 13-14, 13-15, 13-16, 13-17, 13-18, 13-19, 13-20, 13-21, 13-22, 13-23, 13-24, 13-25, 13-26, 13-27, 13-28, 13-29, 13-30, 13-31, 13-32, 13-33, 13-34, 13-35, 13-36, 13-37, 13-38) (e.g., 14-15, 14-16, 14-17, 14-18, 14-19, 14-20, 14-21, 14-22, 14-23, 14-24, 14-25, 14-26, 14-27, 14-28, 14-29, 14-30, 14-31, 14-32, 14-33, 14-34, 14-35, 14-36, 14-37, 14-38) (e.g., 15-16, 15-17, 15-18, 15-19, 15-20, 15-21, 15-22, 15-23, 15-24, 15-25, 15-26, 15-27, 15-28, 15-29, 15-30, 15-31, 15-32, 15-33, 15-34, 15-35, 15-36, 15-37, 15-38) (e.g., 16-17, 16-18, 16-19, 16-20, 16-21, 16-22, 16-23, 16-24, 16-25, 16-26, 16-27, 16-28, 16-29, 16-30, 16-31, 16-32, 16-33, 16-34, 16-35, 16-36, 16-37, 16-38) (e.g., 17-18, 17-19, 17-20, 17-21, 17-22, 17-23, 17-24, 17-25, 17-26, 17-27, 17-28, 17-29, 17-30, 17-31, 17-32, 17-33, 17-34, 17-35, 17-36, 17-37, 17-38) (e.g., 18-19, 18-20, 18-21, 18-22, 18-23, 18-24, 18-25, 18-26, 18-27, 18-28, 18-29, 18-30, 18-31, 18-32, 18-33, 18-34, 18-35, 18-36, 18-37, 18-38) (e.g., 19-20, 19-21, 19-22, 19-23, 19-24, 19-25, 19-26, 19-27, 19-28, 19-29, 19-30, 19-31, 19-32, 19-33, 19-34, 19-35, 19-36, 19-37, 19-38) (e.g., 20-21, 20-22, 20-23, 20-24, 20-25, 20-26, 20-27, 20-28, 20-29, 20-30, 20-31, 20-32, 20-33, 20-34, 20-35, 20-36, 20-37, 20-38) (e.g., 21-22, 21-23, 21-24, 21-25, 21-26, 21-27, 21-28, 21-29, 21-30, 21-31, 21-32, 21-33, 21-34, 21-35, 21-36, 21-37, 21-38) (e.g., 22-23, 22-24, 22-25, 22-26, 22-27, 22-28, 22-29, 22-30, 22-31, 22-32, 22-33, 22-34, 22-35, 22-36, 22-37, 22-38) (e.g., 23-24, 23-25, 23-26, 23-27, 23-28, 23-29, 23-30, 23-31, 23-32, 23-33, 23-34, 23-35, 23-36, 23-37, 23-38) (e.g., 24-25, 24-26, 24-27, 24-28, 24-29, 24-30, 24-31, 24-32, 24-33, 24-34, 24-35, 24-36, 24-37, 24-38) (e.g., 25-26, 25-27, 25-28, 25-29, 25-30, 25-31, 25-32, 25-33, 25-34, 25-35, 25-36, 25-37, 25-38) (e.g., 26-27, 26-28, 26-29, 26-30, 26-31, 26-32, 26-33, 26-34, 26-35, 26-36, 26-37, 26-38) (e.g., 27-28, 27-29, 27-30, 27-31, 27-32, 27-33, 27-34, 27-35, 27-36, 27-37, 27-38) (e.g., 28-29, 28-30, 28-31, 28-32, 28-33, 28-34, 28-35, 28-36, 28-37, 28-38) (e.g., 29-30, 29-31, 29-32, 29-33, 29-34, 29-35, 29-36, 29-37, 29-38) (e.g., 30-31, 30-32, 30-33, 30-34, 30-35, 30-36, 30-37, 30-38) (e.g., 31-32, 31-33, 31-34, 31-35, 31-36, 31-37, 31-38) (e.g., 32-33, 32-34, 32-35, 32-36, 32-37, 32-38) (e.g., 33-34, 33-35, 33-36, 33-37, 33-38) (e.g., 34-35, 34-36, 34-37, 34-38) (e.g., 35-36, 35-37, 35-38) (e.g., 36-37, 36-38) (e.g., 37-38) (e.g., 38 or fewer; 37 or fewer; 36 or fewer; 35 or fewer; 34 or fewer; 33 or fewer; 32 or fewer; 31 or fewer; 30 or fewer; 29 or fewer; 28 or fewer; 27 or fewer; 26 or fewer; 25 or fewer; 24 or fewer; 23 or fewer; 22 or fewer; 21 or fewer; 20 or fewer; 19 or fewer; 18 or fewer; 17 or fewer; 16 or fewer; 15 or fewer; 14 or fewer; 13 or fewer; 12 or fewer; 11 or fewer; 10 or fewer; 9 or fewer; 8 or fewer; 7 or fewer; 6 or fewer; 5 or fewer; 4 or fewer; 3 or fewer; 2 or 1).

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” encompasses the coding regions of a structural gene and includes the sequences located adjacent to the coding region on both the 5′ and 3′ ends, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. As would be understood by one of ordinary skill in the art based on the present disclosure, one or more CpG sites in a DMR can be located in a coding region of a gene, a non-coding regulator region of a gene, or a non-coding region that is not known to be associated with a particular gene, such as a region comprising a long non-coding RNA (lncRNA). In some embodiments, sequences corresponding to these regions can be obtained using an accession number (see, e.g., Table 1) corresponding to a genomic database (e.g., GenBank, NCBI/Ensembl, UniProt, etc.). In some embodiments, one or more CpG sites in a DMR can be located in a genomic region that is unannotated. As provided further herein, unannotated genomic regions comprising one or more CpG sites in a DMR can be described using SEQ ID NOs (see, e.g., Table 1; SEQ ID NOs: 1-6).

As would be recognized by one of ordinary skill in the art based on the present disclosure, the location of one or more CpG sites within a gene or region (e.g., CpG island) and its relevance to a disease or condition can be determined using a variety of techniques, including but not limited to, those disclosed in Chen et al., “Methods for identifying differentially methylated regions for sequence- and array-based data,” Briefings in Functional Genomics, Volume 15, Issue 6, November 2016, Pages 485-490, which is herein incorporated by reference in its entirety and for all purposes.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “wild-type” when made in reference to a protein refers to a protein that has the characteristics of a naturally occurring protein. The term “naturally-occurring” as applied to an object refers to the fact that an object can be found in nature. For example, a polypeptide or polynucleotide sequence that is present in an organism (including viruses) that can be isolated from a source in nature, and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population, or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method. In some embodiments, the primer pair is specific for a differentially methylated region (e.g., DMRs in Tables 1, 2, and 3) and specifically binds at least a portion of a genetic region comprising the DMR.

The term “probe” refers to an oligonucleotide (e.g., a sequence of nucleotides), whether occurring naturally as in a purified restriction digest or produced synthetically, recombinantly, or by PCR amplification, which is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the embodiments of the present disclosure may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the various embodiment of the present disclosure be limited to any particular detection system or label.

The term “target,” as used herein refers to a nucleic acid sought to be sorted out from other nucleic acids, e.g., by probe binding, amplification, isolation, capture, etc. For example, when used in reference to the polymerase chain reaction, “target” refers to the region of nucleic acid bounded by the primers used for polymerase chain reaction, while when used in an assay in which target DNA is not amplified, e.g., in some embodiments of an invasive cleavage assay, a target comprises the site at which a probe and invasive oligonucleotides (e.g., INVADER oligonucleotide) bind to form an invasive cleavage structure, such that the presence of the target nucleic acid can be detected. A “segment” is defined as a region of nucleic acid within the target sequence.

Accordingly, as used herein, “non-target”, e.g., as it is used to describe a nucleic acid such as a DNA, refers to nucleic acid that may be present in a reaction, but that is not the subject of detection or characterization by the reaction. In some embodiments, non-target nucleic acid may refer to nucleic acid present in a sample that does not, e.g., contain a target sequence, while in some embodiments, non-target may refer to exogenous nucleic acid, i.e., nucleic acid that does not originate from a sample containing or suspected of containing a target nucleic acid, and that is added to a reaction, e.g., to normalize the activity of an enzyme (e.g., polymerase) to reduce variability in the performance of the enzyme in the reaction.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

As used herein, the term “amplification reagents” refers to those reagents (deoxyribonucleoside triphosphates, buffer, etc.), needed for amplification except for primers, nucleic acid template, and the amplification enzyme. Typically, amplification reagents along with other reaction components are placed and contained in a reaction vessel.

As used herein, the term “control” when used in reference to nucleic acid detection or analysis refers to a nucleic acid having known features (e.g., known sequence, known copy-number per cell), for use in comparison to an experimental target (e.g., a nucleic acid of unknown concentration). A control may be an endogenous, preferably invariant gene against which a test or target nucleic acid in an assay can be normalized. Such normalizing controls for sample-to-sample variations that may occur in, for example, sample processing, assay efficiency, etc., and allows accurate sample-to-sample data comparison. Genes that find use for normalizing nucleic acid detection assays on human samples include, e.g., b-actin, ZDHHC1, and B3GALT6 (see, e.g., U.S. patent application Ser. Nos 14/966,617 and 62/364,082, each incorporated herein by reference). As used herein “ZDHHC1” refers to a gene encoding a protein characterized as a zinc finger, DHHC-type containing 1, located in human DNA on Chr 16 (16q22.1) and belonging to the DHHC palmitoyltransferase family.

Controls may also be external. For example, in quantitative assays such as qPCR, QuARTS, etc., a “calibrator” or “calibration control” is a nucleic acid of known sequence, e.g., having the same sequence as a portion of an experimental target nucleic acid, and a known concentration or series of concentrations (e.g., a serially diluted control target for generation of calibration curved in quantitative PCR). Typically, calibration controls are analyzed using the same reagents and reaction conditions as are used on an experimental DNA. In certain embodiments, the measurement of the calibrators is done at the same time, e.g., in the same thermal cycler, as the experimental assay. In preferred embodiments, multiple calibrators may be included in a single plasmid, such that the different calibrator sequences are easily provided in equimolar amounts. In particularly preferred embodiments, plasmid calibrators are digested, e.g., with one or more restriction enzymes, to release calibrator portion from the plasmid vector. See, e.g., WO 2015/066695, which is included herein by reference.

As used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state,” “methylation profile,” and “methylation status” of a nucleic acid molecule refers to the presence or absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

As used herein, the term “methylation level” as applied to a methylation marker refers to the amount of methylation within a particular methylation marker. Methylation level may also refer to the amount of methylation within a particular methylation marker in comparison with an established norm or control. Methylation level may also refer to whether one or more cytosine residues present in a CpG context have or do not have a methylation group. Methylation level may also refer to the fraction of cells in a sample that do or do not have a methylation group on such cytosines. Methylation level may also alternatively describe whether a single CpG di-nucleotide is methylated.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.). A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids or by comparing TET-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

The term “methylation score” as used herein is a score indicative of detected methylation events in a marker or panel of markers in comparison with median methylation events for the marker or panel of markers from a random population of mammals (e.g., a random population of 10, 20, 30, 40, 50, 100, or 500 mammals) that do not have a specific neoplasm of interest. An elevated methylation score in a marker or panel of markers can be any score provided that the score is greater than a corresponding reference score. For example, an elevated score of methylation in a marker or panel of markers can be 0.5, 1, 2, 3, 4, 5, 6, 7, 8, 9, 10, or more fold greater than the reference methylation score.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state,” “methylation profile,” and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation,” whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who do not have recurrence. Differential methylation and specific levels or patterns of DNA methylation are prognostic and predictive biomarkers, e.g., once the correct cut-off or predictive characteristics have been defined. In some embodiments, one or more CpG sites in a DMR can be located in non-coding regions, such as regions corresponding to long non-coding RNAs (lncRNAs).

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g., Antequera et al. (1990) Cell 62: 503-514).

As used herein, a “CpG island” or “cytosine-phosphate-guanine island”) refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a “methylation-specific reagent” refers to a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. Examples of such reagents include, but are not limited to, a methylation-sensitive restriction enzyme, a methylation-dependent restriction enzyme, a bisulfite reagent, a TET enzyme, and a borane reducing agent.

A change in the nucleic acid nucleotide sequence by a methylation-specific reagent can also result in a nucleic acid molecule in which each methylated nucleotide is modified to a different nucleotide.

The term “methylation assay” refers to any assay for determining the methylation state of one or more CpG dinucleotide sequences within a sequence of a nucleic acid.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, as described by Gonzalgo et al. (1997) Cancer Research 57: 594-599.

The term “MethyLight™” refers to the art-recognized fluorescence-based real-time PCR technique described by Eads et al. (1999) Cancer Res. 59: 2302-2306.

The term “HeavyMethyl™” refers to an assay wherein methylation specific blocking probes (also referred to herein as blockers) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones (1997) Nucleic Acids Res. 25: 2529-2531.

The term “MSP” (Methylation-specific PCR) refers to the art-recognized methylation assay described by Herman et al. (1996) Proc. Natl. Acad. Sci. USA 93: 9821-9826, and by U.S. Pat. No. 5,786,146.

The term “COBRA” (Combined Bisulfite Restriction Analysis) refers to the art-recognized methylation assay described by Xiong & Laird (1997) Nucleic Acids Res. 25: 2532-2534.

The term “MCA” (Methylated CpG Island Amplification) refers to the methylation assay described by Toyota et al. (1999) Cancer Res. 59: 2307-12, and in WO 00/26401A1.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The term “methylation-specific restriction enzyme” refers to a restriction enzyme that selectively digests a nucleic acid dependent on the methylation state of its recognition site. In the case of a restriction enzyme that specifically cuts if the recognition site is not methylated or is hemi-methylated (a methylation-sensitive enzyme), the cut will not take place (or will take place with a significantly reduced efficiency) if the recognition site is methylated on one or both strands. In the case of a restriction enzyme that specifically cuts only if the recognition site is methylated (a methylation-dependent enzyme), the cut will not take place (or will take place with a significantly reduced efficiency) if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein the terms “copy number variation,” “CNV,” “copy number aberration,” and “CNA,” generally refer to variation in the number of copies of a nucleic acid sequence present in a test sample in comparison with the copy number of the nucleic acid sequence present in a reference sample. In some cases, the nucleic acid sequence is a whole chromosome or significant portion thereof. A “copy number variant” refers to the sequence of nucleic acid in which copy-number differences are found by comparison of a nucleic acid sequence of interest in test sample with an expected level of the nucleic acid sequence of interest. For example, the level of the nucleic acid sequence of interest in the test sample is compared to that present in a qualified sample. Copy number variants/variations include deletions, including microdeletions, insertions, including microinsertions, duplications, multiplications, and translocations. CNVs encompass chromosomal aneuploidies, partial aneuploidies, polyploidies, and partial polyploidies. In some cases, analyzing a nucleic acid sample for CNV refers to characterizing the status of a chromosomal or segment aneuploidy by one of three types of calls (e.g., normal or unaffected, affected, and no-call.) Thresholds for calling normal and affected are typically set. A parameter related to aneuploidy or other copy number variation can be measured in a sample and the measured value can be compared to the thresholds. For example, for duplication type aneuploidies, a call of affected is made if a chromosome or segment dose (or other measured value sequence content) is above a defined threshold set for affected samples. For such aneuploidies, a call of normal is made if the chromosome or segment dose is below a threshold set for normal samples. By contrast for deletion type aneuploidies, a call of affected is made if a chromosome or segment dose is below a defined threshold for affected samples, and a call of normal is made if the chromosome or segment dose is above a threshold set for normal samples. For example, in the presence of trisomy the “normal” call is determined by the value of a parameter, e.g., a test chromosome dose that is below a user-defined threshold of reliability, and the “affected” call is determined by a parameter, e.g., a test chromosome dose, that is above a user-defined threshold of reliability. A “no-call” result is determined by a parameter, e.g., a test chromosome dose that lies between the thresholds for making a “normal” or an “affected” call. The term “no-call” is used interchangeably with “unclassified.”

The term “aneuploidy” herein generally refers to an imbalance of genetic material, for instance caused by a loss or gain of a whole chromosome, or part of a chromosome. The terms “partial aneuploidy” and “partial chromosomal aneuploidy” herein refer to an imbalance of genetic material caused by a loss or gain of part of a chromosome, e.g., partial monosomy and partial trisomy, and encompasses imbalances resulting from translocations, deletions and insertions. The terms “chromosomal aneuploidy” and “complete chromosomal aneuploidy” herein refer to an imbalance of genetic material caused by a loss or gain of a whole chromosome, and includes germline aneuploidy and mosaic aneuploidy. For example, aneuploidy can result from an extra set of chromosomes (e.g., meiosis errors), which can lead to congenital disorders. This type of aneuploidy can be caused by a failure of the chromosomes to separate properly during meiosis, or by a sperm fertilizing an egg with more than one set of chromosomes. In other cases, aneuploidy results from chromosome instability (CIN) due to failure of the mitotic checkpoint, resulting in mis-segregation of chromosomes (e.g., mitosis errors), which leads to gains of oncogenes or loss of tumor suppressors in cancer-related disease states.

As described herein, the term “aneuploidy score” or “AS” generally refers to the total number of altered chromosomal arms in a sample, ranging from 0 (no arms) to 39 (all arms—long and short arms for each non-acrocentric chromosome, and only long arms for chromosomes 13, 14, 15, 21, and 22). As described further herein, the calculated aneuploidy score (AS) for each sample is the total number of chromosomal arm-level gains and losses, adjusted for ploidy.

As used herein, the “sensitivity” of a given marker (or set of markers used together) refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker (or set of markers used together) refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease), and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

The term “AUC” as used herein is an abbreviation for the “area under a curve”. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better; the optimum is 1; a random test would have a ROC curve lying on the diagonal with an area of 0.5; for reference: J. P. Egan. (1975) Signal Detection Theory and ROC Analysis, Academic Press, New York).

The term “neoplasm” as used herein refers to any new and abnormal growth of tissue. Thus, a neoplasm can be a premalignant neoplasm or a malignant neoplasm.

The term “neoplasm-specific marker,” as used herein, refers to any biological material or element that can be used to indicate the presence of a neoplasm. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid that are markers may be referred to, e.g., as “marker genes,” “marker regions,” “marker sequences,” “marker loci,” etc.

As used herein, the term “adenoma” refers to a benign tumor of glandular origin. Although these growths are benign, over time they may progress to become malignant (e.g., esophageal adenocarcinoma or EAC).

The term “pre-cancerous” or “pre-neoplastic” and equivalents thereof refer to any cellular proliferative disorder that is undergoing malignant transformation. For example, as described further herein, low grade dysplastic (LGD) BE and high grade dysplastic (HGD) BE are considered to be pre-cancerous conditions.

As used herein, the term “esophageal disorder” refers to types of disorder associated with the esophagus and/or esophageal tissue. Examples of esophageal disorders include, but are not limited to, Barrett's Esophagus (BE), non-dysplastic Barrett's Esophagus (NDBE), Barrett's esophageal dysplasia (BED), Barrett's esophageal low grade dysplasia (BE-LGD), Barrett's esophageal high-grade dysplasia (BE-HGD), and esophageal adenocarcinoma (EAC).

A “site” of a neoplasm, adenoma, cancer, etc. is the tissue, organ, cell type, anatomical area, body part, etc. in a subject's body where the neoplasm, adenoma, cancer, etc. is located.

As used herein, a “diagnostic” test application includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic test can be used for detecting the presence or likelihood of a subject contracting a neoplasm or the likelihood that such a subject will respond favorably to a compound (e.g., a pharmaceutical, e.g., a drug) or other treatment.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include a given DNA sequence (e.g., a gene) found on the host cell chromosome in proximity to neighboring genes, RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, be combined with other nucleic acids or molecules. For example, an isolated nucleic acid may be present in a host cell into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution containing salts (e.g., NaCl), detergents (e.g., SDS), and other components (e.g., Denhardt's solution, dry milk, salmon sperm DNA, etc.).

The term “sample” is used in its broadest sense. In one sense it can refer to an animal cell or tissue. In another sense, it refers to a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the various embodiments of the present disclosure.

As used herein, a “remote sample” as used in some contexts relates to a sample indirectly collected from a site that is not the cell, tissue, or organ source of the sample. For instance, when sample material originating from the pancreas is assessed in a stool sample the sample is a remote sample.

As used herein, the terms “patient” or “subject” refer to organisms to be subject to various tests described herein. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present disclosure provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including racehorses), and the like.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc. “Allele frequency information” refers to facts or data pertaining to allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### 2. METHYLATED DNA MARKERS AND BIOMARKER PANELS

A whole genome methylation approach was used to simultaneously query copy number aberration (CNA) and DNA methylation to discriminate non-dysplastic Barrett's Esophagus (NDBE) from high grade dysplastic Barrett's Esophagus (HGD-BE) or esophageal adenocarcinoma (EAC), with the potential to act as adjuncts for endoscopic histological surveillance. As described further herein, the present disclosure provides the materials and methods for utilizing sequencing reads from a genome that has been modified in a methylation-specific manner, for instance a cytosine or 5-methylcytosine converted genome, to make copy number aberration (CNA) determinations, including polyploidy and aneuploidy (e.g., determine an aneuploidy score). Most currently available technologies use direct NGS on wild-type, unconverted DNA. However, embodiments of the present disclosure include the ability to perform methylation and CNV analysis from the same chemistry/dataset, without the need to split samples to perform each respective analysis separately. That is, methylation analysis and CNV analysis can be performed simultaneously on the same converted DNA sample. In accordance with these embodiments, the methylation profile in the at least one DMR and the CNV and/or the AS can be determined using the same DNA sample obtained from a subject. In some embodiments, the methylation profile in the at least one DMR and the CNV and/or the AS can be determined using a single DNA sample obtained from the subject. In some embodiments, the sample (e.g., the same sample or the single sample) has been treated with a reagent that modifies DNA in a methylation-specific manner.

Thus, as described further herein, embodiments of the present disclosure provide methods, compositions, and systems for screening various types of esophageal disorders in a biological sample. In accordance with these embodiments, the present disclosure includes, but is not limited to, methods and compositions for detecting the presence of esophageal cancer or pre-cancer from a biological sample. In some embodiments, the biological sample is a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample. In some embodiments, the tissue sample is an esophageal tissue sample. In some embodiments, the esophageal sample is obtained from an esophageal biopsy, or by swabbing, brushing, or using a sponge capsule device. In some embodiments, the subject is a human.

As described further herein, embodiments of the present disclosure include novel differentially methylated regions (DMRs), each individually capable of distinguishing esophageal cancer or pre-cancer from control or benign tissue. In some embodiments, the novel DMRs are capable of distinguishing high grade dysplastic Barrett's Esophagus (HGD-BE) or esophageal adenocarcinoma (EAC) from non-dysplastic Barrett's Esophagus (NDBE) or a normal esophageal control. In accordance with these embodiments, the novel DMR(s) is from a gene selected from ACVRL1, ADAMTS8, ADAP2, ADRBK2, AKR1B1, ANK1, ANKRD13B, ANXA6, ARNT2, B4GALNT2, BACH2, BCL11A, BSCL2, C12orf53, C14orf82, C17orf107, C18orf1, C1orf95, C5orf42, CACNA1C, CAMK1D, CAMTA1, CBX6, CCDC85A, CCKBR, CD38, CDKN2A, CH25H, CHST1, CHST15, CNTLN, CRHR1, CRTC1, CXCR4, CYP1B1, DCTN2, DIDO1, DMKN, DSE, DYNC1I1, EML6, ENOX1, EPHA4, ESRRG, FAM176A, FAM78B, FBXO10, FERMT2, FHOD3, FLJ45079, FMNL1, FOXP2, FRMD4B, FZD8, GALNTL1, GALNTL4, GAS1, GBGT1, GLIPR2, GNAI1, GNAL, GPR37, GRASP, GRID1, GRM8, GSC, HAR1A, HCN2, HEY2, HIST1H2BE, HS3ST3B1, HTR7, IGFBP2, INHA, INSRR, IRX3, ISM2, KCNG3, KCNK4, KCNS2, KCTD15, KIAA1522, KIAA1614, KIF26A, KL, KLF15, KLHL10, KRT77, KSR2, LBH, LMX1A, LMX1B, LOC100526820, LONRF2, LRFN2, LRRN1, MAF, MAFB, MARK1, ADAMTSL4-AS1, PGBD5, HSPA12A, SFTPD, LOC107984507, LOC100128253, CISTR, SLC16A7, CTXND1, MAX.chr15.4912, ZNF423, RBFOX1, LOC105376772, GSE1, MAX.chr17.8070, ZNF709_6125, MAX.chr19.3439, GREB1, CYP27C1, AC068134.6, LOC388780, STOX2_8441, PPARGC1A, MAX.chr4.4552, PDZD2, MAX.chr6.6743, LPAL2, CDK14, MAX.chr8.3003, MCOLN2, MEGF11, MFSD11, MRC2, MSX1, NAT8L, NAV1, NBEA, NCRNA00092, NECAB2, NEURL, NGEF, NHLH2, NKD1, NLGN1, NOG, NPPC, NR3C1, NRXN2, NTN1, OBSCN, OCA2, OSBPL1A, OXR1, P2RY1, PDE2A, PDE9A, PDGFRA, PID1, PLCG2, PLCL1, PNPLA3, POU3F1, POU3F2, PRKACB, PRKAR2B, PRKG1, PRR18, PRR5L, PTHLH, PYGL, RARG, RHBDL3, RIMS2, ROR2, RPRML, SDK2, SOX9, STOX2_6730, SYNGR1, TAC4, TNFRSF19, TRANK1, TSPAN33, TSPAN4, TSPAN5, UBE2E2, UCHL1, UNC5A, VASH2, VIM, WNT6, ZBTB10, ZNF680, ZNF709_4918, ZNF738, ZNF808, and ZNF845 (Table 1), including any combinations thereof. In some embodiments, the novel DMR(s) is from any gene selected from Table 1, including any combinations thereof. Each novel DMR alone is capable of distinguishing HGD-BE and/or EAC from NDBE and/or a control sample, and combining two or more of the novel DMRs can provide increased sensitivity. Therefore, combinations of two or more novel DMRs selected from Table 1 are provided.

Embodiments of the present disclosure also include novel differentially methylated regions (DMRs), each individually capable of distinguishing esophageal cancer or pre-cancer from control or benign tissue. In some embodiments, the novel DMRs are capable of distinguishing high grade dysplastic Barrett's Esophagus (HGD-BE) or esophageal adenocarcinoma (EAC) from non-dysplastic Barrett's Esophagus (NDBE) or a normal esophageal control. In accordance with these embodiments, the novel DMR(s) is from a gene selected ACVRL1, ADAMTS8, ADAP2, ADRBK2, AKR1B1, ANK1, ANKRD13B, ANXA6, ARNT2, B4GALNT2, BACH2, BCL11A, BSCL2, C14orf82, C18orf1, C1orf95, C5orf42, CAMK1D, CAMTA1, CCDC85A, CD38, CDKN2A, CHST1, CHST15, CRHR1, CYP1B1, DIDO1, DSE, DYNC1I1, EML6, ENOX1, EPHA4, FAM176A, FBXO10, FERMT2, FHOD3, FLJ45079, FMNL1, FRMD4B, FZD8, GALNTL1, GALNTL4, GAS1, GBGT1, GLIPR2, GNAI1, GNAL, GRASP, GRID1, GRM8, GSC, HAR1A, HCN2, HEY2, HIST1H2BE, HS3ST3B1, HTR7, IGFBP2, INHA, IRX3, ISM2, KCNG3, KCNK4, KCNS2, KCTD15, KIAA1614, KL, KLHL10, KSR2, LBH, LMX1A, LMX1B, LOC100526820, LONRF2, LRFN2, MAFB, MARK1, PGBD5, HSPA12A, SFTPD, LOC107984507, LOC100128253, CISTR, SLC16A7, CTXND1, ZNF423, LOC105376772, GSE1, MAX.chr19.3439, GREB1, CYP27C1, AC068134.6, LOC388780, STOX2_8441, PPARGC1A, PDZD2, MAX.chr6.6743, LPAL2, CDK14, MCOLN2, MEGF11, MRC2, MSX1, NAT8L, NAV1, NBEA, NCRNA00092, NEURL, NGEF, NHLH2, NKD1, NLGN1, NOG, NPPC, NR3C1, NRXN2, OBSCN, OCA2, OSBPL1A, OXR1, P2RY1, PDE2A, PDGFRA, PID1, PLCG2, PLCL1, PNPLA3, POU3F1, POU3F2, PRKACB, PRKAR2B, PRKG1, PRR18, PRR5L, PTHLH, PYGL, RHBDL3, RIMS2, ROR2, RPRML, SDK2, SOX9, STOX2_6730, SYNGR1, TAC4, TRANK1, TSPAN4, UBE2E2, UCHL1, UNC5A, VASH2, ZBTB10, ZNF680, ZNF709_4918, ZNF738, ZNF808, and ZNF845 (Table 2), including any combinations thereof. In some embodiments, the novel DMR(s) is from any gene selected from Table 2, including any combinations thereof. Each novel DMR alone is capable of distinguishing HGD-BE and/or EAC from NDBE and/or a control sample, and combining two or more of the novel DMRs can provide increased sensitivity. Therefore, combinations of two or more novel DMRs selected from Table 2 are provided.

Embodiments of the present disclosure also include novel differentially methylated regions (DMRs), each individually capable of distinguishing esophageal cancer or pre-cancer from control or benign tissue. In some embodiments, the novel DMRs are capable of distinguishing high grade dysplastic Barrett's Esophagus (HGD-BE) or esophageal adenocarcinoma (EAC) from non-dysplastic Barrett's Esophagus (NDBE) or a normal esophageal control. In accordance with these embodiments, the novel DMR(s) is from a gene selected from BACH2, C5orf42, FHOD3, HIST1H2BE, IRX3, KIAA1614, LONRF2, MAFB, PDGFRA, PID1, POU3F1, PRR5L, RHBDL3, and SDK2 (Table 3), including any combinations thereof. In some embodiments, the novel DMR(s) is from any gene selected from Table 3, including any combinations thereof. Each novel DMR alone is capable of distinguishing HGD-BE and/or EAC from NDBE and/or a control sample, and combining two or more of the novel DMRs can provide increased sensitivity. Therefore, combinations of two or more novel DMRs selected from Table 3 are provided.

Embodiments of the present disclosure also include novel differentially methylated regions (DMRs), each individually capable of distinguishing esophageal cancer or pre-cancer from control or benign tissue. In some embodiments, the novel DMRs are capable of distinguishing high grade dysplastic Barrett's Esophagus (HGD-BE) or esophageal adenocarcinoma (EAC) from non-dysplastic Barrett's Esophagus (NDBE) or a normal esophageal control. In accordance with these embodiments, the novel DMR(s) is from a gene selected from KL, PGBD5, ROR2, and LMX1B (Example 4), including any combinations thereof. In some embodiments, the novel DMR(s) is from any gene selected from Example 4, including any combinations thereof. Each novel DMR alone is capable of distinguishing HGD-BE and/or EAC from NDBE and/or a control sample, and combining two or more of the novel DMRs can provide increased sensitivity. Therefore, combinations of two or more novel DMRs selected from Example 4 are provided.

In accordance with the above, a control sample comprises a sample from a subject that does not have cancer, a sample from a subject that does not have esophageal cancer, a sample from a subject that does not have esophageal pre-cancer, or a sample from a subject that has a type of cancer that is not an esophageal cancer or pre-cancer. In some embodiments, a control sample comprises a sample from a subject that has non-dysplastic Barrett's Esophagus (NDBE). In some embodiments, the control sample is from a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and a stool sample. In some embodiments, the tissue sample is an esophageal tissue sample. In some embodiments, the esophageal sample is obtained from an esophageal biopsy, or by swabbing, brushing, or using a sponge capsule device.

In some embodiments, the present disclosure provides compositions and methods for identifying, determining, and/or classifying esophageal cancer or pre-cancer from a biological sample (e.g., a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample). The methods generally comprise determining the methylation profile of at least one methylation marker in a biological sample isolated from a subject. In some embodiments, a change in the methylation state or profile of the marker is indicative of the presence, class, or site of esophageal cancer or pre-cancer. Generally, such methods are useful for the detection of the presence or absence of esophageal cancer (e.g., esophageal adenocarcinoma (EAC)) or pre-cancer (e.g., high grade dysplastic Barrett's Esophagus (HGD-BE)).

In some embodiments, methods are provided that comprise contacting a nucleic acid (e.g., genomic DNA) in a biological sample obtained from a subject with at least one reagent or series of reagents that distinguishes between methylated and non-methylated nucleotides (e.g., CpG dinucleotides) within at least one methylation marker; and detecting for the presence or absence of esophageal cancer or pre-cancer (e.g., afforded with a sensitivity of greater than or equal to 80% and a specificity of greater than or equal to 80%).

In some embodiments, methods are provided that comprise measuring one or both of a methylation level for one or more genes or methylated DNA markers in a biological sample from a human individual through treating genomic DNA in the biological sample with a reagent that modifies DNA in a methylation-specific manner; and determining the methylation level of the one or more genes or methylation markers.

In some embodiments, methods are provided that comprise measuring an amount of one or more methylated DNA markers or genes in DNA from a biological sample; measuring an amount of at least one reference marker in the DNA; and calculating a value for the amount of the at least one methylated marker gene measured in the DNA as a percentage of the amount of the reference marker gene measured in the DNA, wherein the value indicates the amount of the at least one methylated marker DNA measured in the biological sample.

In some embodiments, methods are provided that comprise measuring a methylation level of a CpG site for one or more genes in a biological sample of a human individual through treating genomic DNA in the biological sample with bisulfite a reagent capable of modifying DNA in a methylation-specific manner; amplifying the modified genomic DNA using a set of primers for the selected one or more genes; and determining the methylation level of the CpG site for the selected one or more genes.

In some embodiments, the present disclosure provides methods for characterizing a biological sample comprising measuring one or both of a methylation level of a CpG site for one or more genes in a biological sample of a human individual through treating genomic DNA in the biological sample with bisulfite; amplifying the bisulfite-treated genomic DNA using a set of primers for the selected one or more genes; and determining the methylation level of the CpG site. In some embodiments, the method comprises comparing one or both of the methylation level of a methylation marker to a methylation level of a corresponding set of genes in control samples without a specific type of cancer; and/or determining that a subject has an esophageal cancer or pre-cancer when one or both of the methylation level measured in the one or more genes is higher than the methylation level measured in the respective control samples.

In some embodiments, the present disclosure provides methods comprising one or both of measuring in a biological sample a methylation level of one or more genes or markers through treating genomic DNA in the biological sample with bisulfite; amplifying the bisulfite-treated genomic DNA using a set of primers for the selected one or more genes; and determining the methylation level of the one or more genes or markers.

In some embodiments, the present disclosure provides methods of screening for esophageal cancer or pre-cancer in a sample obtained from a subject. In accordance with these embodiments, the method includes one or both of assaying a methylation state or profile of one or more methylated DNA markers; and identifying the subject as having esophageal cancer or pre-cancer when the methylation state or profile of the marker is different than a methylation state or profile of the marker assayed in a subject that does not have esophageal cancer or pre-cancer.

In some embodiments, the present disclosure provides methods that comprise measuring a methylation level for one or more genes or markers in a biological sample of a human individual through treating genomic DNA in the biological sample with a reagent that modifies DNA in a methylation-specific manner; amplifying the treated genomic DNA using a set of primers for the selected one or more genes or markers; and determining the methylation level of the one or more genes or markers.

In some embodiments, the present disclosure provides methods for characterizing a biological sample comprising measuring an amount of at least one methylated DNA marker in DNA extracted from the biological sample; treating genomic DNA in the biological sample with bisulfite; amplifying the bisulfite-treated genomic DNA using primers specific for a CpG site for each marker, wherein the primers specific for each marker are capable of binding an amplicon bound by a primer sequence; and determining the methylation level of the CpG site for one or more genes.

In some embodiments, the present disclosure provides methods comprising measuring the methylation level of one or more methylated DNA markers in DNA extracted from a biological sample through extracting genomic DNA from a biological sample of a human individual suspected of having or having esophageal cancer or pre-cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Tables 1 or 2; and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having esophageal cancer or pre-cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 1; and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having esophageal cancer or pre-cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 2; and measuring the methylation level of one or more methylated markers.

In some embodiments, the present disclosure provides methods comprising measuring the methylation level of one or more methylated DNA markers in DNA extracted from a biological sample through extracting genomic DNA from a biological sample of a human individual suspected of having or having esophageal cancer or pre-cancer; treating the extracted genomic DNA with bisulfite, amplifying the bisulfite-treated genomic DNA with primers specific for the one or more markers, wherein the primers specific for the one or more markers are capable of binding at least a portion of the bisulfite-treated genomic DNA for a chromosomal region for the marker recited in Table 3; and measuring the methylation level of one or more methylated markers.

In some embodiments, the present disclosure provides methods comprising extracting genomic DNA from a biological sample of a human individual suspected of having or having cancer, treating the extracted genomic DNA with bisulfite, amplifying the bisulfite-treated genomic DNA using separate primers specific for CpG sites for one or more of the methylated DNA markers, and measuring a methylation level of the CpG site for each of the one or more markers.

As described further herein, embodiments of the present disclosure include methods and compositions for characterizing a biological sample and determining a methylation profile in at least one differentially methylated region (DMR) of a DNA sample obtained from a subject having or suspected of having an esophageal cancer or pre-cancer by treating the sample with a reagent that modifies DNA in a methylation-specific manner. In some embodiments, the method includes detecting the presence of esophageal cancer or pre-cancer from a biological sample. In some embodiments, the at least one DMR is capable of distinguishing high grade dysplastic Barrett's Esophagus (HGD-BE) or esophageal adenocarcinoma (EAC) from non-dysplastic Barrett's Esophagus (NDBE) or a normal esophageal control.

In accordance with these embodiments, the method further includes assessing the DNA sample from the subject for copy number variations (CNVs) or copy number aberrations (CNAs). In some embodiments, the CNV discriminates between a subject having or suspected of having high grade dysplastic Barrett's Esophagus or esophageal adenocarcinoma (EAC) and a control DNA sample. In some embodiments, the at least one DMR comprises an increased CNV compared to a control DNA sample.

In some embodiments, the method includes determining an aneuploidy score (AS) for the DNA sample from the subject. In some embodiments, the AS discriminates between a subject having or suspected of having high grade dysplastic Barrett's Esophagus or esophageal adenocarcinoma (EAC) and a control DNA sample. In some embodiments, the at least one DMR comprises an increased aneuploidy score as compared to a control DNA sample. As described herein, an aneuploidy score refers to the total number of altered chromosomal arms in a sample, and determining an aneuploidy score can provide an additional and/or alternative method for assessing the DNA sample for an esophageal cancer or pre-cancer as compared to a control.

In some embodiments, assessing CNV and/or determining an aneuploidy score can complement a methylation profile determination for a DNA sample obtained from a subject having or suspected of having an esophageal cancer or pre-cancer. In some embodiments, assessing CNV or determining an aneuploidy score involves obtaining sequencing reads from the same cytosine-converted genome used to assess methylation profile of a DNA sample. Whereas most currently methods use direct NGS on unconverted DNA. However, in the methods of the present disclosure, methylation and CNV/aneuploidy readouts were obtained simultaneously from the same chemistry/dataset.

As would be appreciated by one of ordinary skill in the art based on the present disclosure, the various methods described herein are not limited to the use of any one specific methylated DNA markers, methylated marker genes, methylated genes, and/or DMRs. That is, one or more of the methylated DNA markers, methylated marker genes, methylated genes, and/or DMRs of the present disclosure can be used to distinguish and/or identify esophageal cancer or pre-cancer from a control, including any combinations thereof. Additionally, the methylated DNA markers, methylated marker genes, methylated genes, and/or DMRs of the present disclosure can comprise a region or subregion (e.g., a gene on a chromosome, a single nucleotide, a CpG island, etc.) of any of the markers listed in Tables 1, 2, and 3.

In some embodiments, the DMR is from a gene selected from ACVRL1, ADAMTS8, ADAP2, ADRBK2, AKR1B1, ANK1, ANKRD13B, ANXA6, ARNT2, B4GALNT2, BACH2, BCL11A, BSCL2, C12orf53, C14orf82, C17orf107, C18orf1, C1orf95, C5orf42, CACNA1C, CAMK1D, CAMTA1, CBX6, CCDC85A, CCKBR, CD38, CDKN2A, CH25H, CHST1, CHST15, CNTLN, CRHR1, CRTC1, CXCR4, CYP1B1, DCTN2, DIDO1, DMKN, DSE, DYNC1I1, EML6, ENOX1, EPHA4, ESRRG, FAM176A, FAM78B, FBXO10, FERMT2, FHOD3, FLJ45079, FMNL1, FOXP2, FRMD4B, FZD8, GALNTL1, GALNTL4, GAS1, GBGT1, GLIPR2, GNAI1, GNAL, GPR37, GRASP, GRID1, GRM8, GSC, HAR1A, HCN2, HEY2, HIST1H2BE, HS3ST3B1, HTR7, IGFBP2, INHA, INSRR, IRX3, ISM2, KCNG3, KCNK4, KCNS2, KCTD15, KIAA1522, KIAA1614, KIF26A, KL, KLF15, KLHL10, KRT77, KSR2, LBH, LMX1A, LMX1B, LOC100526820, LONRF2, LRFN2, LRRN1, MAF, MAFB, MARK1, ADAMTSL4-AS1, PGBD5, HSPA12A, SFTPD, LOC107984507, LOC100128253, CISTR, SLC16A7, CTXND1, MAX.chr15.4912, ZNF423, RBFOX1, LOC105376772, GSE1, MAX.chr17.8070, ZNF709_6125, MAX.chr19.3439, GREB1, CYP27C1, AC068134.6, LOC388780, STOX2_8441, PPARGC1A, MAX.chr4.4552, PDZD2, MAX.chr6.6743, LPAL2, CDK14, MAX.chr8.3003, MCOLN2, MEGF11, MFSD11, MRC2, MSX1, NAT8L, NAV1, NBEA, NCRNA00092, NECAB2, NEURL, NGEF, NHLH2, NKD1, NLGN1, NOG, NPPC, NR3C1, NRXN2, NTN1, OBSCN, OCA2, OSBPL1A, OXR1, P2RY1, PDE2A, PDE9A, PDGFRA, PID1, PLCG2, PLCL1, PNPLA3, POU3F1, POU3F2, PRKACB, PRKAR2B, PRKG1, PRR18, PRR5L, PTHLH, PYGL, RARG, RHBDL3, RIMS2, ROR2, RPRML, SDK2, SOX9, STOX2_6730, SYNGR1, TAC4, TNFRSF19, TRANK1, TSPAN33, TSPAN4, TSPAN5, UBE2E2, UCHL1, UNC5A, VASH2, VIM, WNT6, ZBTB10, ZNF680, ZNF709_4918, ZNF738, ZNF808, and ZNF845 (Table 1); and the subject has or is suspected of having an esophageal cancer (e.g., esophageal adenocarcinoma (EAC)) or an esophageal pre-cancer (e.g., high grade dysplastic Barrett's Esophagus (HGD-BE)). In some embodiments, determining the methylation profile of the DMR comprises comparing the methylation profile to a corresponding region from a control DNA sample (e.g., non-dysplastic Barrett's Esophagus (NDBE) or a normal esophageal control).

In some embodiments, the DMR is from a gene selected from ACVRL1, ADAMTS8, ADAP2, ADRBK2, AKR1B1, ANK1, ANKRD13B, ANXA6, ARNT2, B4GALNT2, BACH2, BCL11A, BSCL2, C14orf82, C18orf1, C1orf95, C5orf42, CAMK1D, CAMTA1, CCDC85A, CD38, CDKN2A, CHST1, CHST15, CRHR1, CYP1B1, DIDO1, DSE, DYNC1I1, EML6, ENOX1, EPHA4, FAM176A, FBXO10, FERMT2, FHOD3, FLJ45079, FMNL1, FRMD4B, FZD8, GALNTL1, GALNTL4, GAS1, GBGT1, GLIPR2, GNAI1, GNAL, GRASP, GRID1, GRM8, GSC, HAR1A, HCN2, HEY2, HIST1H2BE, HS3ST3B1, HTR7, IGFBP2, INHA, IRX3, ISM2, KCNG3, KCNK4, KCNS2, KCTD15, KIAA1614, KL, KLHL10, KSR2, LBH, LMX1A, LMX1B, LOC100526820, LONRF2, LRFN2, MAFB, MARK1, PGBD5, HSPA12A, SFTPD, LOC107984507, LOC100128253, CISTR, SLC16A7, CTXND1, ZNF423, LOC105376772, GSE1, MAX.chr19.3439, GREB1, CYP27C1, AC068134.6, LOC388780, STOX2_8441, PPARGC1A, PDZD2, MAX.chr6.6743, LPAL2, CDK14, MCOLN2, MEGF11, MRC2, MSX1, NAT8L, NAV1, NBEA, NCRNA00092, NEURL, NGEF, NHLH2, NKD1, NLGN1, NOG, NPPC, NR3C1, NRXN2, OBSCN, OCA2, OSBPL1A, OXR1, P2RY1, PDE2A, PDGFRA, PID1, PLCG2, PLCL1, PNPLA3, POU3F1, POU3F2, PRKACB, PRKAR2B, PRKG1, PRR18, PRR5L, PTHLH, PYGL, RHBDL3, RIMS2, ROR2, RPRML, SDK2, SOX9, STOX2_6730, SYNGR1, TAC4, TRANK1, TSPAN4, UBE2E2, UCHL1, UNC5A, VASH2, ZBTB10, ZNF680, ZNF709_4918, ZNF738, ZNF808, and ZNF845 (Table 2); and the subject has or is suspected of having an esophageal cancer (e.g., esophageal adenocarcinoma (EAC)) or an esophageal pre-cancer (e.g., high grade dysplastic Barrett's Esophagus (HGD-BE)). In some embodiments, determining the methylation profile of the DMR comprises comparing the methylation profile to a corresponding region from a control DNA sample (e.g., non-dysplastic Barrett's Esophagus (NDBE) or a normal esophageal control).

In some embodiments, the DMR is from a gene selected from BACH2, C5orf42, FHOD3, HIST1H2BE, IRX3, KIAA1614, LONRF2, MAFB, PDGFRA, PID1, POU3F1, PRR5L, RHBDL3, and SDK2 (Table 3); and the subject has or is suspected of having an esophageal cancer (e.g., esophageal adenocarcinoma (EAC)) or an esophageal pre-cancer (e.g., high grade dysplastic Barrett's Esophagus (HGD-BE)). In some embodiments, determining the methylation profile of the DMR comprises comparing the methylation profile to a corresponding region from a control DNA sample (e.g., non-dysplastic Barrett's Esophagus (NDBE) or a normal esophageal control).

In some embodiments, the novel DMR(s) capable of distinguishing esophageal cancer or pre-cancer from a control sample is associated with an area under a ROC curve (AUC) greater than or equal to 0.5, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.6, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.7, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.75, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.8, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.85, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.9, wherein the ROC curve discriminates between a subject having or suspected of having esophageal cancer or pre-cancer and a control DNA sample. In some embodiments, the novel DMR(s) capable of distinguishing esophageal cancer or pre-cancer from a control sample is associated with an area under a ROC curve (AUC) greater than or equal to 0.95, wherein the ROC curve discriminates between a subject having or suspected of having esophageal cancer or pre-cancer and a control DNA sample.

In some embodiments, the novel DMR(s) capable of distinguishing esophageal cancer or pre-cancer from a control sample comprises an increased methylation percentage as compared to a control DNA sample. In some embodiments, the novel DMR(s) capable of distinguishing esophageal cancer or pre-cancer from a control sample comprises an increased hypermethylation ratio as compared to a control DNA sample.

In some embodiments, determining the methylation profile of at least one DMR comprises amplifying at least a portion of the DMR using a set of primers. In some embodiments, determining the methylation profile of at least one DMR comprises performing at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR. In some embodiments, determining the methylation profile of at least one DMR comprises determining the presence or absence of methylation at a CpG site. In some embodiments, the one or more CpG sites are present in a coding region, a non-coding region, and/or a regulatory region of a gene (e.g., any one of the genes disclosed herein). In some embodiments, the DMR(s) capable of distinguishing an esophageal cancer or pre-cancer from a control sample can be validated using at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR. In some embodiments, the DMR(s) capable of distinguishing an esophageal cancer or pre-cancer from a control sample can be assessed based on at least one of an area under a ROC curve (AUC), fold-change in methylation, methylation percentage, and/or hypermethylation ratio between a test sample and a control sample.

As one of ordinary skill in the art would understand based on the present disclosure, esophageal cancer or pre-cancer can be predicted by various combinations of markers (e.g., as identified by statistical techniques related to specificity and sensitivity of prediction). Embodiments of the present disclosure provide methods for identifying predictive combinations and validated predictive combinations for esophageal cancer or pre-cancer.

Such methods are not limited to a particular manner or technique for determining characterizing, measuring, or assaying methylation for one or more methylated markers, methylated marker genes, genes, DMRs, and/or DNA methylated markers. In some embodiments, such techniques are based upon an analysis of the methylation status (e.g., CpG methylation status) of at least one marker, region of a marker, or base of a marker comprising a DMR.

In some embodiments, measuring the methylation state or profile of a methylated DNA marker in a sample comprises determining the methylation state of one nucleotide base. In some embodiments, measuring the methylation state of a methylated DNA marker in the sample comprises determining the extent of methylation at a plurality of nucleotide bases. Moreover, in some embodiments, the methylation state or profile of a methylated DNA marker comprises an increase in methylation of the marker relative to a normal methylation state or profile of the marker. In some embodiments, the methylation state or profile of the marker comprises decreased methylation of the marker relative to a normal methylation state of the marker. In some embodiments the methylation state or profile of the marker comprises a different pattern of methylation of the marker relative to a normal methylation state or profile of the marker.

Furthermore, in some embodiments the marker is a region of 100 or fewer nucleotide bases. In some embodiments, the marker is a region of 500 or fewer nucleotide bases. In some embodiments, the marker is a region of 1000 or fewer nucleotide bases. In some embodiments, the marker is a region of 5000 or fewer nucleotide bases. In some embodiments, the marker is one nucleotide base. In some embodiments, the marker is in a high CpG density promoter region.

In certain embodiments, methods for analyzing a nucleic acid for the presence of 5-methylcytosine involves treatment of DNA with a reagent that modifies DNA in a methylation-specific manner. Examples of such reagents include, but are not limited to, a methylation-sensitive restriction enzyme, a methylation-dependent restriction enzyme, a bisulfite reagent, a TET enzyme, and a borane reducing agent.

A frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16: 431-36; Grigg G, DNA Seq. (1996) 6: 189-98),methylation-specific PCR (MSP) as is disclosed, e.g., in U.S. Pat. No. 5,786,146, or using an assay comprising sequence-specific probe cleavage, e.g., a QuARTS flap endonuclease assay (see, e.g., Zou et al. (2010) “Sensitive quantification of methylated markers with a novel methylation specific technology” Clin Chem 56: A199; and in U.S. Pat. Nos. 8,361,720; 8,715,937; 8,916,344; and 9,212,392.

In some embodiments, conventional techniques include methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26: 2255.

The bisulfite technique typically involves amplifying short, specific fragments of a known nucleic acid subsequent to a bisulfite treatment, then assaying the product by sequencing (Olek & Walter (1997) Nat. Genet. 17: 275-6) or using a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures can be used in conjunction with bisulfite treatment according to embodiments of the present disclosure. These assays allow for determination of the methylation state of one or a plurality of CpG dinucleotides (e.g., CpG islands) within a nucleic acid sequence. Such assays involve, among other techniques, sequencing of bisulfite-treated nucleic acid, PCR (for sequence-specific amplification), Southern blot analysis, and use of methylation-specific restriction enzymes, e.g., methylation-sensitive or methylation-dependent enzymes.

For example, genomic sequencing has been simplified for analysis of methylation patterns and 5-methylcytosine distributions by using bisulfite treatment (Frommer et al. (1992) Proc. Natl. Acad. Sci. USA 89: 1827-1831). Additionally, restriction enzyme digestion of PCR products amplified from bisulfite-converted DNA finds use in assessing methylation state, e.g., as described by Sadri & Hornsby (1997) Nucl. Acids Res. 24: 5058-5059 or as embodied in the method known as COBRA (Combined Bisulfite Restriction Analysis) (Xiong & Laird (1997) Nucleic Acids Res. 25: 2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, DMR, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include DNA denaturation buffer; sulfonation buffer; DNA recovery reagents or kits (e.g., precipitation, ultrafiltration, affinity column); desulfonation buffer; and DNA recovery components.

Assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bisulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase.

MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to methylated and unmethylated PCR primers for specific loci (e.g., specific genes, markers, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMan®) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g., a “TaqMan®” probe, a Lightcycler® probe, etc.) For example, in some applications double-stranded genomic DNA is treated with sodium bisulfite and subjected to one of two sets of PCR reactions using TaqMan® probes, e.g., with MSP primers and/or HeavyMethyl blocker oligonucleotides and a TaqMan® probe. The TaqMan® probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can be used with any suitable probe, e.g., “TaqMan®” probes, Lightcycler® probes, in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as MspI) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as MspI, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7: 133-6; Meissner et al. (2005) “Reduced representation bisulfite sequencing for comparative high-resolution DNA methylation analysis” Nucleic Acids Res. 33: 5868-77.

In some embodiments, a quantitative allele-specific real-time target and signal amplification (QuARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes a 5′ nuclease, e.g., a FEN-1 endonuclease, to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophores per flap, providing exponential signal amplification. QuARTS can detect multiple targets in a single reaction well by using FRET cassettes with different dyes. See, e.g., in Zou et al. (2010) “Sensitive quantification of methylated markers with a novel methylation specific technology” Clin Chem 56: A199), and U.S. Pat. Nos. 8,361,720; 8,715,937; 8,916,344; and 9,212,392, each of which is incorporated herein by reference for all purposes.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivates thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in WO 2013/116375.

In some embodiments, fragments of the treated DNA are amplified using sets of primer oligonucleotides and an amplification enzyme, according to the method and compositions described herein. The amplification of several DNA segments can be carried out simultaneously in one and the same reaction vessel. Typically, the amplification is carried out using a polymerase chain reaction (PCR). Amplicons are typically 100 to 2000 base pairs in length.

In some embodiments of the method, the methylation status or profile of CpG positions within or near a differentially methylated region (e.g., Tables 1, 2, and 3) may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primer pairs contain at least one primer that hybridizes to a bisulfite treated CpG dinucleotide. Therefore, the sequence of said primers comprises at least one CpG dinucleotide. MSP primers specific for non-methylated DNA contain a “T” at the position of the C position in the CpG.

Such methods are not limited to a specific type or kind of primer or primer pair related to the one or more methylated markers, methylated marker genes, genes, DMRs, and/or methylated DNA markers. In some embodiments, the primer or primer pair specific for each methylated marker gene are capable of binding an amplicon bound by a primer sequence, wherein the amplicon bound by the primer sequence for the marker gene is at least a portion of a genetic region for the methylated marker gene recited in Tables 1, 2, or 3. In some embodiments, the primer or primer pair for a methylated marker is a set of primers that specifically binds at least a portion of a genetic region comprising the methylated marker recited in Tables 1, 2, or 3.

In another embodiment, the present disclosure provides a method for converting an oxidized 5-methylcytosine residue in cell-free DNA to a dihydrouracil residue (see, Liu et al., 2019, Nat Biotechnol. 37, pp. 424-429; U.S. Patent Application Publication No. 202000370114). The method involves reaction of an oxidized 5mC residue selected from 5-formylcytosine (5fC), 5-carboxymethylcytosine (5caC), and combinations thereof, with a borane reducing agent. The oxidized 5mC residue may be naturally occurring or, more typically, the result of a prior oxidation of a 5mC or 5hmC residue, e.g., oxidation of 5mC or 5hmC with a TET family enzyme (e.g., TET1, TET2, or TET3), or chemical oxidation of 5 mC or 5hmC, e.g., with potassium perruthenate (KRuO₄) or an inorganic peroxo compound or composition such as peroxotungstate (see, e.g., Okamoto et al. (2011) Chem. Commun. 47:11231-33) and a copper (II) perchlorate/2,2,6,6-tetramethylpiperidine-1-oxyl (TEMPO) combination (see Matsushita et al. (2017) Chem. Commun. 53:5756-59).

The borane reducing agent may be characterized as a complex of borane and a nitrogen-containing compound selected from nitrogen heterocycles and tertiary amines. The nitrogen heterocycle may be monocyclic, bicyclic, or polycyclic, but is typically monocyclic, in the form of a 5- or 6-membered ring that contains a nitrogen heteroatom and optionally one or more additional heteroatoms selected from N, O, and S. The nitrogen heterocycle may be aromatic or alicyclic. Preferred nitrogen heterocycles herein include 2-pyrroline, 2H-pyrrole, 1H-pyrrole, pyrazolidine, imidazolidine, 2-pyrazoline, 2-imidazoline, pyrazole, imidazole, 1,2,4-triazole, 1,2,4-triazole, pyridazine, pyrimidine, pyrazine, 1,2,4-triazine, and 1,3,5-triazine, any of which may be unsubstituted or substituted with one or more non-hydrogen substituents. Typical non-hydrogen substituents are alkyl groups, particularly lower alkyl groups, such as methyl, ethyl, n-propyl, isopropyl, n-butyl, isobutyl, t-butyl, and the like. Exemplary compounds include pyridine borane, 2-methylpyridine borane (also referred to as 2-picoline borane), and 5-ethyl-2-pyridine.

The reaction of the borane reducing agent with the oxidized 5mC residue in cell-free DNA is advantageous insofar as non-toxic reagents and mild reaction conditions can be employed; there is no need for any bisulfate, nor for any other potentially DNA-degrading reagents. Furthermore, conversion of an oxidized 5mC residue to dihydrouracil with the borane reducing agent can be carried out without need for isolation of any intermediates, in a “one-pot” or “one-tube” reaction. This is quite significant, since the conversion involves multiple steps, i.e., (1) reduction of the alkene bond linking C-4 and C-5 in the oxidized 5mC, (2) deamination, and (3) either decarboxylation, if the oxidized 5mC is 5caC, or deformylation, if the oxidized 5mC is 5fC.

In addition to a method for converting an oxidized 5-methylcytosine residue in cell-free DNA to a dihydrouracil residue, the present disclosure also provides a reaction mixture related to the aforementioned method. The reaction mixture comprises a sample of cell-free DNA containing at least one oxidized 5-methylcytosine residue selected from 5caC, 5fC, and combinations thereof, and a borane reducing agent effective to effective to reduce, deaminate, and either decarboxylate or deformylate the at least one oxidized 5-methylcytosine residue. The borane reducing agent is a complex of borane and a nitrogen-containing compound selected from nitrogen heterocycles and tertiary amines, as explained above. In a preferred embodiment, the reaction mixture is substantially free of bisulfite, meaning substantially free of bisulfite ion and bisulfite salts. Ideally, the reaction mixture contains no bisulfite.

In a related aspect of the present disclosure, a kit is provided for converting 5mC residues in cell-free DNA to dihydrouracil residues, where the kit includes a reagent for blocking 5hmC residues, a reagent for oxidizing 5mC residues beyond hydroxymethylation to provide oxidized 5mC residues, and a borane reducing agent effective to reduce, deaminate, and either decarboxylate or deformylate the oxidized 5mC residues. The kit may also include instructions for using the components to carry out the above-described method.

In another embodiment, a method is provided that makes use of the above-described oxidation reaction. The method enables detecting the presence and location of 5-methylcytosine residues in cell-free DNA, and comprises the following steps: (a) modifying 5hmC residues in fragmented, adapter-ligated cell-free DNA to provide an affinity tag thereon, wherein the affinity tag enables removal of modified 5hmC-containing DNA from the cell-free DNA; (b) removing the modified 5hmC-containing DNA from the cell-free DNA, leaving DNA containing unmodified 5mC residues; (c) oxidizing the unmodified 5mC residues to give DNA containing oxidized 5mC residues selected from 5caC, 5fC, and combinations thereof, (d) contacting the DNA containing oxidized 5mC residues with a borane reducing agent effective to reduce, deaminate, and either decarboxylate or deformylate the oxidized 5mC residues, thereby providing DNA containing dihydrouracil residues in place of the oxidized 5mC residues; (e) amplifying and sequencing the DNA containing dihydrouracil residues; (f) determining a 5-methylation pattern from the sequencing results in (e).

In some embodiments, the present disclosure provides a method for identifying 5-methylcytosine (5mC) or 5-hydroxymethylcytosine (5hmC) in a target nucleic acid. In some embodiments, the method comprises providing a biological sample comprising the target nucleic acid, modifying the target nucleic acid by converting the 5mC and 5hmC in the nucleic acid sample to 5-carboxylcytosine (5caC) and/or 5-formylcytosine (5fC) by contacting the nucleic acid sample with a TET enzyme so that one or more 5caC or 5fC residues are generated, and converting the 5caC and/or 5fC to dihydrouracil (DHU) by treating the target nucleic acid with a borane reducing agent to provide a modified nucleic acid sample comprising a modified target nucleic acid, and detecting the sequence of the modified target nucleic acid; wherein a cytosine (C) to thymine (T) transition or a cytosine (C) to DHU transition in the sequence of the modified target nucleic acid compared to the target nucleic acid provides the location of either a 5mC or 5hmC in the target nucleic acid. In some embodiments, the borane reducing agent is 2-picoline borane.

In some embodiments, detecting the sequence of the modified target nucleic acid comprises one or more of chain termination sequencing, microarray, high-throughput sequencing, and restriction enzyme analysis. In some embodiments, the TET enzyme is selected from the group consisting of human TET1, TET2, and TET3; murine TET1, TET2, and TET3; Naegleria TET (NgTET); and Coprinopsis cinerea (CcTET). In some embodiments, the method further comprises a step of blocking one or more modified cytosines. In some embodiments, the step of blocking comprises adding a sugar to a 5hmC. In some embodiments, the method further comprises a step of amplifying the copy number of one or more nucleic acid sequences. In some embodiments, the oxidizing agent is potassium perruthenate or Cu(II)/TEMPO (2,2,6,6-tetramethylpiperidine-1-oxyl.)

The cell-free DNA is typically extracted from a biological sample from a subject, where the sample can be whole blood, buffy coat, plasma, urine, saliva, mucosal excretions, organ secretions, sputum, stool, or tears. In some embodiments, the cell-free DNA is derived from a tumor (e.g., an esophageal tumor). In other embodiments, the cell-free DNA is from a patient with a disease or other pathogenic condition. The cell-free DNA may or may not be derived from a tumor. In some embodiments, the cell-free DNA in which 5hmC residues are to be modified is in purified, fragmented form, and adapter-ligated. DNA purification in this context can be carried out using any suitable method known to those of ordinary skill in the art and/or described in the pertinent literature, and, while cell-free DNA can itself be highly fragmented, further fragmentation may occasionally be desirable, as described, for example, in U.S. Patent Publication No. 2017/0253924. The cell-free DNA fragments are generally in the size range of about 20 nucleotides to about 500 nucleotides, more typically in the range of about 20 nucleotides to about 250 nucleotides. The purified cell-free DNA fragments that are modified in step (a) have been end-repaired using conventional means (e.g., a restriction enzyme) so that the fragments have a blunt end at each 3′ and 5′ terminus. In a preferred method, as described in WO 2017/176630, the blunted fragments have also been provided with a 3′ overhang comprising a single adenine residue using a polymerase such as Taq polymerase. This facilitates subsequent ligation of a selected universal adapter, i.e., an adapter such as a Y-adapter or a hairpin adapter that ligates to both ends of the cell-free DNA fragments and contains at least one molecular barcode. Use of adapters also enables selective PCR enrichment of adapter-ligated DNA fragments.

In some embodiments, the “purified, fragmented cell-free DNA” comprises adapter-ligated DNA fragments. Modification of 5hmC residues in these cell-free DNA fragments with an affinity tag is done so as to enable subsequent removal of the modified 5hmC-containing DNA from the cell-free DNA. In one embodiment, the affinity tag comprises a biotin moiety, such as biotin, desthiobiotin, oxybiotin, 2-iminobiotin, diaminobiotin, biotin sulfoxide, biocytin, or the like. Use of a biotin moiety as the affinity tag allows for facile removal with streptavidin (e.g., streptavidin beads, magnetic streptavidin beads, etc.).

Tagging 5hmC residues with a biotin moiety or other affinity tag is accomplished by covalent attachment of a chemoselective group to 5hmC residues in the DNA fragments, where the chemoselective group is capable of undergoing reaction with a functionalized affinity tag so as to link the affinity tag to the 5hmC residues. In one embodiment, the chemoselective group is UDP glucose-6-azide, which undergoes a spontaneous 1,3-cycloaddition reaction with an alkyne-functionalized biotin moiety, as described in Robertson et al. (2011) Biochem. Biophys. Res. Comm. 411(1):40-3, U.S. Pat. No. 8,741,567, and WO 2017/176630. Addition of an alkyne-functionalized biotin-moiety thus results in covalent attachment of the biotin moiety to each 5hmC residue.

The affinity-tagged DNA fragments can then be pulled down using, in one embodiment, streptavidin, in the form of streptavidin beads, magnetic streptavidin beads, or the like, and set aside for later analysis, if so desired. The supernatant remaining after removal of the affinity-tagged fragments contains DNA with unmodified 5mC residues and no 5hmC residues.

In some embodiments, the unmodified 5mC residues are oxidized to provide 5caC residues and/or 5fC residues, using any suitable means. The oxidizing agent is selected to oxidize 5mC residues beyond hydroxymethylation, i.e., to provide 5caC and/or 5fC residues. Oxidation may be carried out enzymatically, using a catalytically active TET family enzyme. A “TET family enzyme” or a “TET enzyme” as those terms are used herein refer to a catalytically active “TET family protein” or a “TET catalytically active fragment” as defined in U.S. Pat. No. 9,115,386, the disclosure of which is incorporated by reference herein. A preferred TET enzyme in this context is TET2; see Ito et al. (2011) Science 333(6047):1300-1303. Oxidation may also be carried out chemically, as described in the preceding section, using a chemical oxidizing agent. Examples of suitable oxidizing agent include, without limitation: a perruthenate anion in the form of an inorganic or organic perruthenate salt, including metal perruthenates such as potassium perruthenate (KRuO₄), tetraalkylammonium perruthenates such as tetrapropylammonium perruthenate (TPAP) and tetrabutylammonium perruthenate (TBAP), and polymer supported perruthenate (PSP); and inorganic peroxo compounds and compositions such as peroxotungstate or a copper (II) perchlorate/TEMPO combination. It is unnecessary at this point to separate 5fC-containing fragments from 5caC-containing fragments, insofar as in the next step of the process, converts both 5fC residues and 5caC residues to dihydrouracil (DHU).

In some embodiments, 5-hydroxymethylcytosine residues are blocked with β-glucosyltransferase (β3GT), while 5-methylcytosine residues are oxidized with a TET enzyme effective to provide a mixture of 5-formylcytosine and 5-carboxymethylcytosine. The mixture containing both of these oxidized species can be reacted with 2-picoline borane or another borane reducing agent to give dihydrouracil. In a variation on this embodiment, 5hmC-containing fragments are not removed. Rather, “TET-Assisted Picoline Borane Sequencing (TAPS),” 5mC-containing fragments and 5hmC-containing fragments are together enzymatically oxidized to provide 5fC- and 5caC-containing fragments. Reaction with 2-picoline borane results in DHU residues wherever 5mC and 5hmC residues were originally present. “Chemical Assisted Picoline Borane Sequencing (CAPS),” involves selective oxidation of 5hmC-containing fragments with potassium perruthenate, leaving 5mC residues unchanged.

As disclosed in International PCT Appln. PCT/US2019/012627, incorporated herein by reference in its entirety, TAPS comprises the use of mild enzymatic and chemical reactions to detect 5mC and 5hmC directly and quantitatively at base-resolution without affecting unmodified cytosines. In a related embodiment, the above method further includes identifying a hydroxymethylation pattern in the 5hmC-containing DNA removed from the cell-free DNA. This can be carried out using the techniques described in detail in WO 2017/176630. The process can be carried out without removal or isolation of intermediates in a one-tube method. For example, initially, cell-free DNA fragments, preferably adapter-ligated DNA fragments, are subjected to functionalization with βGT-catalyzed uridine diphosphoglucose 6-azide, followed by biotinylation via the chemoselective azide groups. This procedure results in covalently attached biotin at each 5hmC site. In a next step, the biotinylated strands and strands containing unmodified (native) 5mC are pulled down simultaneously for further processing. The native 5mC-containing strands are pulled down using an anti-5mC antibody or a methyl-CpG-binding domain (MBD) protein, as is known in the art. Then, with the 5hmC residues blocked, the unmodified 5mC residues are selectively oxidized using any suitable technique for converting 5mC to 5fC and/or 5caC, as described elsewhere herein.

The fragments obtained by means of the amplification can carry a directly or indirectly detectable label. In some embodiments, the labels are fluorescent labels, radionuclides, or detachable molecule fragments having a typical mass that can be detected in a mass spectrometer. Where said labels are mass labels, some embodiments provide that the labeled amplicons have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

In some embodiments, the markers described herein find use in QUARTS assays performed on stool samples. In some embodiments, methods for producing DNA samples and, in particular, to methods for producing DNA samples that comprise highly purified, low-abundance nucleic acids in a small volume (e.g., less than 100, less than 60 microliters) and that are substantially and/or effectively free of substances that inhibit assays used to test the DNA samples (e.g., PCR, INVADER, QuARTS assays, etc.) are provided. Such DNA samples find use in diagnostic assays that qualitatively detect the presence of, or quantitatively measure the activity, expression, or amount of, a gene, a gene variant (e.g., an allele), or a gene modification (e.g., methylation) present in a sample taken from a patient. For example, some cancers are correlated with the presence of particular mutant alleles or particular methylation states, and thus detecting and/or quantifying such mutant alleles or methylation states has predictive value in the diagnosis and treatment of cancer.

Many valuable genetic markers are present in extremely low amounts in samples and many of the events that produce such markers are rare. Consequently, even sensitive detection methods such as PCR require a large amount of DNA to provide enough of a low-abundance target to meet or supersede the detection threshold of the assay. Moreover, the presence of even low amounts of inhibitory substances can compromise the accuracy and precision of these assays directed to detecting such low amounts of a target. Accordingly, provided herein are methods providing the requisite management of volume and concentration to produce such DNA samples.

In some embodiments, the biological sample is a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample. In some embodiments, the tissue sample is an esophageal tissue sample. In some embodiments, the tissue sample is an endoscopic esophageal brushing sample. In some embodiments, the sample includes esophageal tissue, and/or the sample is obtained through endoscopic brushing or nonendoscopic whole esophageal brushing or swabbing using a tethered device (e.g., such as a capsule sponge, balloon, or other device).

In some embodiments, the sample comprises a tissue and/or biological fluid obtained from a human patient. In some embodiments, the sample comprises esophageal tissue. In some embodiments, the sample comprises esophageal tissue obtained through whole esophageal swabbing or brushing. In some embodiments, the sample comprises a secretion. In some embodiments, the sample comprises blood, serum, plasma, gastric secretions, pancreatic juice, a gastrointestinal biopsy sample, microdissected cells from an esophageal biopsy, esophageal cells sloughed into the gastrointestinal lumen, and/or esophageal cells recovered from stool. These samples may originate from the upper gastrointestinal tract, the lower gastrointestinal tract, or comprise cells, tissues, and/or secretions from both the upper gastrointestinal tract and the lower gastrointestinal tract. In some embodiments, the sample comprises cellular fluid, ascites, urine, feces, pancreatic fluid, fluid obtained during endoscopy, blood, mucus, or saliva. In some embodiments, the sample is a stool sample. Such samples can be obtained by any number of means known in the art, such as will be apparent to the skilled person. For instance, urine and fecal samples are easily attainable, while blood, ascites, serum, or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. In some embodiments, the sample is obtained through esophageal swabbing or brushing or use of a sponge capsule device.

Such samples can be obtained by any number of means known in the art, such as will be apparent to the skilled person. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. The technology is not limited in the methods used to prepare the samples and provide a nucleic acid for testing. For example, in some embodiments, a DNA is isolated from a sample (e.g., a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample) using direct gene capture, e.g., as detailed in U.S. Pat. Nos. 8,808,990 and 9,169,511, and in WO 2012/155072, or by a related method.

The analysis of markers can be carried out separately or simultaneously with additional markers within one test sample. For example, several markers can be combined into one test for efficient processing of multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

Genomic DNA may be isolated by any means, including the use of commercially available kits. Briefly, wherein the DNA of interest is encapsulated by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types comprising neoplastic matter or pre-neoplastic matter are suitable for use in the present method, e.g., cell lines, histological slides, biopsies, endoscopic brushing sample, paraffin-embedded tissue, body fluids, stool, tissue, colonic effluent, urine, blood plasma, blood serum, whole blood, buffy coat, isolated blood cells, cells isolated from the blood, and combinations thereof.

The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Appl. Ser. No. 61/485,386 or by a related method.

The genomic DNA sample is then treated with at least one reagent, or series of reagents, which distinguishes between methylated and non-methylated CpG dinucleotides within at least one marker comprising a DMR (e.g., DMRs Tables 1, 2, or 3).

In some embodiments, the reagent converts cytosine bases which are unmethylated at the 5′-position to uracil, thymine, or another base which is dissimilar to cytosine in terms of hybridization behavior. However, in some embodiments, the reagent may be a methylation sensitive restriction enzyme.

In some embodiments, the genomic DNA sample is treated in such a manner that cytosine bases that are unmethylated at the 5′ position are converted to uracil, thymine, or another base that is dissimilar to cytosine in terms of hybridization behavior. In some embodiments, this treatment is carried out with bisulfite (e.g., hydrogen sulfite, disulfite) followed by alkaline hydrolysis.

The treated nucleic acid is then analyzed to determine the methylation state of the target gene sequences (at least one gene, genomic sequence, or nucleotide from a marker comprising a DMR, e.g., at least one DMR chosen from the DMRs in Tables 1, 2, or 3). The method of analysis may be selected from those known in the art, including those listed herein, e.g., QuARTS and MSP as described herein.

Such samples can be obtained by any number of means known in the art, such as will be apparent to the skilled person. For instance, urine and fecal samples are easily attainable, while blood, ascites, serum, or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens.

### 3. METHODS OF TREATMENT

In some embodiments, the present disclosure provides methods for treating a subject (e.g., a patient having or suspected of having esophageal cancer or pre-cancer). In accordance with these embodiments, the method includes determining a methylation state or profile of one or more methylated DNA markers provided herein, and administering a treatment to the patient based on the results of determining the methylation state. In some embodiments, the method includes assessing the DNA sample from the subject for copy number variations (CNVs) or copy number aberrations (CNAs), and administering a treatment to the patient based on the results of the assessment. In some embodiments, the method includes assessing an aneuploidy score (AS) in the DNA sample from the subject, and administering a treatment to the patient based on the results of the assessment.

The treatment may be administration of a pharmaceutical compound, a vaccine, performing a surgery, imaging the patient, performing another test. In some embodiments, treating a subject includes a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments, a method for diagnosing a specific type of cancer in a subject is provided. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example one or more biomarkers (e.g., one or more methylated markers, methylated marker genes, genes, DMRs, and/or DNA methylated markers as disclosed herein), the methylation state of which is indicative of the presence, severity, or absence of the condition. In some embodiments, a diagnosis can be made on the basis of one or more diagnostic indicators, such as for example CNV or aneuploidy, which can be indicative of the presence, severity, or absence of the condition (e.g., esophageal cancer or pre-cancer).

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment (e.g., determining methylation profile and/or aneuploidy score) of cancer biomarkers is useful to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing,” as used herein, is further inclusive of determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on measurement of the diagnostic biomarkers (e.g., DMR) and/or aneuploidy score, as disclosed herein. Further, in some embodiments of the presently disclosed subject matter, multiple determination of the biomarkers over time can be made to facilitate diagnosis and/or prognosis. A temporal change in methylation profile and/or aneuploidy score can be used to predict a clinical outcome, monitor the progression of cancer or a subtype of cancer, and/or monitor the efficacy of appropriate therapies directed against the cancer. In such an embodiment for example, one might expect to see a change in the methylation state of one or more biomarkers (e.g., DMR) and/or the aneuploidy score disclosed herein (and potentially one or more additional biomarker(s), if monitored).

The presently disclosed subject matter further provides in some embodiments a method for determining whether to initiate or continue prophylaxis or treatment of a cancer in a subject. In some embodiments, the method comprises providing a series of biological samples over a time period from the subject; analyzing the series of biological samples to determine a methylation profile and/or aneuploidy score in each of the biological samples; and comparing any measurable change in the methylation profile and/or aneuploidy score in each of the biological samples. Any changes over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation profiles and/or aneuploidy scores can be measured in each of the samples taken from different time points and qualitative and/or quantitative differences noted. A change in the methylation states of the biomarker levels and/or aneuploidy scores from the different samples can be correlated with a specific cancer risk, prognosis, determining treatment efficacy, and/or progression of the cancer in the subject. In some embodiments, the methods and compositions of the present disclosure are for treatment or diagnosis of disease at an early stage, for example, before symptoms of the disease appear. In some embodiments, the methods and compositions of the present disclosure are for treatment or diagnosis of disease at a clinical stage.

In some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker (e.g., a DMR) and/or aneuploidy score. Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., having a normal methylation state of one or more DMR, and/or a normal aneuploidy score), the chance of a given outcome (e.g., suffering from a specific type of cancer) may be very low.

In some embodiments, a statistical analysis associates a prognostic indicator with a predisposition to an adverse outcome. For example, in some embodiments, a methylation profile and/or aneuploidy score different from that in a normal control sample obtained from a patient who does not have a cancer can signal that a subject is more likely to suffer from a cancer than subjects with a level that is more similar to the methylation profile and/or aneuploidy score in the control sample, as determined by a level of statistical significance. Additionally, a change in methylation profile and/or aneuploidy score from a baseline (e.g., “normal”) level can be reflective of subject prognosis, and the degree of change in methylation state and/or aneuploidy score can be related to the severity of adverse events. Statistical significance is often determined by comparing two or more populations and determining a confidence interval and/or ap value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state of a prognostic or diagnostic biomarker disclosed herein (e.g., a DMR) and/or aneuploidy score can be established, and the degree of change in the methylation profile and/or aneuploidy score in a biological sample is simply compared to the threshold degree of change in the methylation profile and/or aneuploidy score. For example, a preferred threshold change in the methylation state for biomarkers provided herein is about 5%, about 10%, about 15%, about 20%, about 25%, about 30%, about 50%, about 75%, about 100%, and about 150%. In yet other embodiments, a “nomogram” can be established, by which a methylation state of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states of a biomarker and/or aneuploidy scores as a function of assay units, e.g., fluorescent signal intensity, if a fluorescent label is used. Using samples taken from multiple donors, standard curves can be provided for control methylation states and/or aneuploidy scores in normal tissue. In certain embodiments of the method, a subject is identified as having cancer upon identifying an aberrant methylation state of one or more DMRs provided herein and/or abnormal aneuploidy scores in a biological sample obtained from the subject. In other embodiments of the method, the detection of an aberrant methylation state of one or more of such biomarkers and/or abnormal aneuploidy scores in a biological sample obtained from the subject results in the subject being identified as having cancer.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

As mentioned above, depending on the embodiment of the method of the present disclosure, detecting a change in methylation profile and/or aneuploidy score can be a qualitative determination or it can be a quantitative determination. As such, the step of diagnosing a subject as having, or at risk of developing, a specific type of cancer indicates that certain threshold measurements are made (e.g., the methylation state of the one or more biomarkers in the biological sample varies from a predetermined control methylation state). In some embodiments of the method, the control methylation profile and/or aneuploidy score is any detectable methylation profile and/or aneuploidy score. In other embodiments of the method where a control sample is tested concurrently with the biological sample, the predetermined methylation profile and/or aneuploidy score is the methylation profile and/or aneuploidy score in the control sample. In other embodiments of the method, the predetermined methylation profile and/or aneuploidy score is based upon and/or identified by a standard curve. In other embodiments of the method, the predetermined methylation profile and/or aneuploidy score is a specific profile or range of profiles. As such, the predetermined methylation profile and/or aneuploidy score can be chosen, within acceptable limits that will be apparent to those skilled in the art, based in part on the embodiment of the method being practiced and the desired specificity, etc.

Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, embodiments of the present disclosure provide for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; and horses. Thus, also provided is the diagnosis and treatment of livestock, including, but not limited to, domesticated swine, ruminants, ungulates, horses (including racehorses), and the like.

### 4. SAMPLES, KITS, AND CONTROLS

Embodiments of the present disclosure provide technology for screening multiple types of esophageal cancer or pre-cancer from a biological sample. In accordance with these embodiments, the present disclosure includes, but is not limited to, methods and compositions for detecting the presence of multiple types and/or subtypes of esophageal cancer or pre-cancer from a biological sample. In some embodiments, the biological sample is a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample. In some embodiments, the tissue sample is an esophageal tissue sample. In some embodiments, the esophageal sample is obtained from an esophageal biopsy, or by swabbing, brushing, or using a sponge capsule device. In some embodiments, the subject is a human.

In other embodiments, “sample,” “test sample,” and “biological sample” refer to fluid sample containing or suspected of containing a methylated DNA marker of the present disclosure. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis. In a particular example, the source is a mammalian (e.g., human) bodily substance (e.g., bodily fluid, blood such as whole blood, buffy coat, serum, plasma, urine, saliva, sweat, sputum, semen, mucus, lacrimal fluid, lymph fluid, amniotic fluid, interstitial fluid, cerebrospinal fluid, feces, tissue, organ, one or more dried blood spots, or the like). Tissues may include, but are not limited to, esophageal tissue, stomach tissue, pancreatic tissue, bile duct/liver tissue, and colorectal tissue. The sample may be a liquid sample or a liquid extract of a solid sample. In some embodiments, the source of the sample may be an organ or tissue, such as a biopsy sample and/or an endoscopic brushing sample (e.g., endoscopic esophageal brushing sample), which may be solubilized by tissue disintegration/cell lysis.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL.

In some cases, the fluid sample may be diluted prior to use in an assay. For example, in embodiments where the source containing a methylated DNA marker is a human body fluid (e.g., blood, serum, secretion), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

In some cases, the sample may undergo pre-analytical processing. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art. In some cases, the fluid sample may be concentrated prior to use in an assay. For example, in embodiments where the source containing a methylated DNA marker is a human body fluid (e.g., blood, serum, secretion), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

It may be desirable to include a control. The control may be analyzed concurrently with the sample from the subject as described above. The results obtained from the subject sample can be compared to the results obtained from the control sample. Standard curves may be provided, with which assay results for the sample may be compared. Such standard curves present levels of one or more methylated DNA markers as a function of assay units. Using samples taken from multiple donors, standard curves can be provided for reference levels of a methylated DNA marker in normal healthy tissue, as well as for “at-risk” levels of the methylated DNA marker in tissue taken from donors, who may have one or more characteristics of an esophageal cancer or pre-cancer.

Embodiments of the present disclosure also include a kit for performing the methods described herein. The kits comprise embodiments of the compositions, devices, apparatuses, etc. described herein, and instructions for use of the kit. Such instructions describe appropriate methods for preparing an analyte from a sample, e.g., for collecting a sample and preparing a nucleic acid from the sample. Individual components of the kit are packaged in appropriate containers and packaging (e.g., vials, boxes, blister packs, ampules, jars, bottles, tubes, and the like) and the components are packaged together in an appropriate container (e.g., a box or boxes) for convenient storage, shipping, and/or use by the user of the kit. It is understood that liquid components (e.g., a buffer) may be provided in a lyophilized form to be reconstituted by the user. Kits may include a control or reference for assessing, validating, and/or assuring the performance of the kit. For example, a kit for assaying the amount of a nucleic acid present in a sample may include a control comprising a known concentration of the same or another nucleic acid for comparison and, in some embodiments, a detection reagent (e.g., a primer) specific for the control nucleic acid. The kits are appropriate for use in a clinical setting and, in some embodiments, for use in a user's home. The components of a kit, in some embodiments, provide the functionalities of a system for preparing a nucleic acid solution from a sample. In some embodiments, certain components of the system are provided by the user.

In some embodiments, the present disclosure provides compositions (e.g., reaction mixtures). In some embodiments, the present disclosure provides a composition comprising a nucleic acid comprising a DMR and a reagent capable of modifying DNA in a methylation-specific manner (e.g., a methylation-sensitive restriction enzyme, a methylation-dependent restriction enzyme, and a bisulfite reagent) (e.g., a methylation-sensitive restriction enzyme, a methylation-dependent restriction enzyme, Ten Eleven Translocation (TET) enzyme (e.g., human TET, human TET2, human TET3, murine TET1, murine TET2, murine TET3, Naegleria TET (NgTET), Coprinopsis cinerea (CcTET)), or a variant thereof), borane reducing agent). Some embodiments provide a composition comprising a nucleic acid comprising a DMR and an oligonucleotide as described herein. Some embodiments provide a composition comprising a nucleic acid comprising a DMR and a methylation-sensitive restriction enzyme. Some embodiments provide a composition comprising a nucleic acid comprising a DMR and a polymerase.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. In some embodiments, a microprocessor is part of a system for determining a methylation profile (e.g., of one or more DMRs in Tables 1, 2, or 3) and/or an aneuploidy score; comparing methylation profile and/or an aneuploidy score; generating standard curves; determining a Ct value; calculating a fraction, frequency, or percentage of methylation; identifying a CpG island; determining a specificity and/or sensitivity of an assay or marker; calculating an ROC curve and an associated AUC; sequence analysis; all as described herein or is known in the art. In some embodiments, a microprocessor is part of a system for determining a methylation profile (e.g., of one or more DMRs in Tables 1, 2, or 3) and/or an aneuploidy score; comparing methylation profile and/or an aneuploidy score; generating standard curves; determining a Ct value; calculating a fraction, frequency, or percentage of methylation; identifying a CpG island; determining a specificity and/or sensitivity of an assay or marker; calculating an ROC curve and an associated AUC; sequence analysis; all as described herein or is known in the art.

In some embodiments, a software or hardware component receives the results of multiple assays and determines a single value result to report to a user that indicates a cancer risk based on the results of the multiple assays (e.g., determining the methylation state of one or more DMRs in Tables 1, 2, or 3, and/or an aneuploidy score). Related embodiments calculate a risk factor based on a mathematical combination (e.g., a weighted combination, a linear combination) of the results from the multiple assays (e.g., determining the methylation state of one or more DMRs in Tables 1, 2, or 3, and/or an aneuploidy score). In some embodiments, the methylation state of a DMR defines a dimension and may have values in a multidimensional space and the coordinate defined by the methylation states of multiple DMRs is a result (e.g., to report to a user, or related to a cancer risk).

The various embodiments of the present disclosure are associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, California and Motorola Corporation of Schaumburg, Illinois. Such processors include, or may be in communication with, media, for example computer-readable media, which stores instructions that, when executed by the processor, cause the processor to perform the steps described herein.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device. All such components, computers, and systems described herein as associated with the technology may be logical or virtual.

In some embodiments, the present disclosure provides systems for screening for esophageal cancer or pre-cancer in a sample obtained from a subject. Exemplary embodiments of systems include, e.g., a system for screening for esophageal cancer or pre-cancer in a sample obtained from a subject (e.g., a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and/or a stool sample). In some embodiments, the system comprises an analysis component configured to determine the methylation state of one or more methylated markers and/or an aneuploidy score in a sample, a software component configured to compare the methylation state of the one or more methylated markers and/or an aneuploidy score in the sample with a control sample or a reference sample recorded in a database, and an alert component configured to alert a user of a cancer associated state.

In some embodiments, an alert is determined by a software component that receives the results from multiple assays (e.g., determining the methylation states of the one or more methylated markers and/or an aneuploidy scores) and calculating a value or result to report based on the multiple results.

Some embodiments provide a database of weighted parameters associated with each methylated marker provided herein for use in calculating a value or result and/or an alert to report to a user (e.g., such as a physician, nurse, clinician, etc.). In some embodiments all results from multiple assays are reported. In some embodiments, one or more results are used to provide a score, value (e.g., aneuploidy score), or result based on a composite of one or more results from multiple assays that is indicative of a cancer risk in a subject. Such methods are not limited to particular methylation markers. In such methods and systems, the one or more methylation markers comprise a base in a DMR selected from the DMRs in Tables 1, 2, and 3.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

The various components of the kit optionally are provided in suitable containers as necessary. The kit can further include containers for holding or storing a sample (e.g., a container or cartridge for a urine, whole blood, buffy coat, plasma, serum sample, tissue, or bodily secretion sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like. In some embodiments, the instrument is a collection device. In some embodiments, the kit includes a collection device for endoscopic brushing or non-endoscopic whole esophageal brushing or swabbing using a tethered device (e.g., such as a capsule sponge, balloon, or other device). In some embodiments, the biological sample is obtained from the subject, and the method further comprises extracting the DNA sample from the biological sample. In some embodiments, the biological sample is collected with a collection device having an absorbing member capable of collecting the biological sample upon contact. In some embodiments, the absorbing member is a sponge configured for insertion into an orifice.

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Experiments were conducted to identify a panel of genetic markers capable of distinguishing non-dysplastic Barrett's Esophagus (NDBE) from precancerous conditions, such as low grade dysplasia (LGD) or high grade dysplasia (HGD), and/or cancerous conditions, such as esophageal adenocarcinoma (EAC), based on differentially methylated regions (DMRs) and/or DNA copy number variation (CNV) in candidate genetic markers.

A reduced representation bisulfite sequencing (RRBS) study was performed on tissue biopsy samples along with a whole genome sequencing (WGS) study on endoscopic brushing samples. Briefly, with respect to the RRBS tissue biopsy study, ~50 million mapped reads were generated per sample on average, and 4 million CpGs were generated per sample at 10× or greater coverage. After employing a proprietary marker discovery pipeline algorithm, logistic analysis, and the initial filtering, as described below in the materials and methods, 4501 DMRs were identified with an average length of 156 bp. These mapped to 1549 annotated genes and 758 non-annotated regions of the genome. Further filtering to remove control sample CpG methylation and imposing a case/control FC cut-off brought the number of annotated and non-annotated regions to 1196. For the endoscopic brushing WGS study, ~500 million mapped deduplicated reads were generated per sample on average, and 24 million CpGs were generated per sample at 10× or greater coverage. A total of 7345 DMRs were identified with an average length of 72 bp. These mapped to 2351 genes and 1374 non-annotated regions. A total of 3534 regions remained after applying subsequent filters and cut-offs. It was expected that the WGS numbers would exceed the RRBS numbers given that RRBS is an enzyme-mediated enrichment of the genome from 3.2 billion bases to less than 5 million. RRBS, due to the size selection step and the MspI enzyme CCGG specificity misses potential CpG island and promoter regions which don't harbor these tetramers in close proximity.

The genomic regions for the 1196 RRBS DMRs were merged with the 3534 WGS DMRs to determine common sites of hypermethylation. Regions were required to overlap or have endpoints within 500 bases of each other. A total of 227 DMRs met these criteria. These were subsequently narrowed down to 156 DMRs. Most of the exclusions were for regions that did not display any substantial concordant CpG methylation, which is a hallmark of bona fide, functional event in methylation mediated tumor suppressor gene inactivation or oncogene, enhancer element activation. Others were for regions which had strong concordant methylation in the NDBE cohort. Some regions were unusually short (e.g., <60 bases) which limits their informativeness and imposes constraints on capture and methylation specific amplification. In addition to the 156 merged regions, 43 additional candidate WGS DMRs were identified which were not in the RRBS data (possibly due to enrichment exclusion step in the protocol). The complete set of 199 DMRs with gene annotations or SEQ ID NOs are listed in Table 1.

TABLE 1
Differentially methylated regions (DMRs) capable of discriminating Barrett's Esophagus
(BE) and/or normal esophageal samples from precancerous high grade dysplasia (HGD)
and/or esophageal adenocarcinoma (EAC). These DMRs were identified from the whole
genome sequencing (WGS) study using esophageal endoscopic brushing samples.

| | | | Accession No. | |
|---|---|---|---|---|
| | | | (NCBI Entrez | |
| DMR | Gene | | Gene/Ensembl) or | Chr. |
| No. | Annotation | Gene Name | SEQ ID NO | No. |
| 1 | ACVRL1 | activin A receptor type II-like 1 | 94 | 12 |
| 2 | ADAMTS8 | ADAM metallopeptidase with | 11095 | 11 |
| | | thrombospondin type 1 motif, 8 | | |
| 3 | ADAP2 | ArfGAP with dual PH domains 2 | 55803 | 17 |
| 4 | ADRBK2 | adrenergic, beta, receptor kinase 2 | 157 | 22 |
| 5 | AKR1B1 | aldo-keto reductase family 1, | 231 | 7 |
| | | member B1 (aldose reductase) | | |
| 6 | ANK1 | ankyrin 1, erythrocytic | 286 | 8 |
| 7 | ANKRD13B | ankyrin repeat domain 13B | 124930 | 17 |
| 8 | ΛNXΛ6 | annexin Λ6 | 309 | 5 |
| 9 | ARNT2 | aryl-hydrocarbon receptor nuclear | 9915 | 15 |
| | | translocator 2 | | |
| 10 | B4GALNT2 | beta-1,4-N-acetyl-galactosaminyl | 124872 | 17 |
| | | transferase 2 | | |
| 11 | BACH2 | BTB and CNC homology 1, basic leucine | 60468 | 6 |
| | | zipper transcription factor 2 | | |
| 12 | BCL11A | B-cell CLL/lymphoma 11A (zinc finger | 53335 | 2 |
| | | protein) | | |
| 13 | BSCL2 | Berardinelli-Seip congenital | 26580 | 11 |
| | | lipodystrophy 2 (seipin) | | |
| 14 | C12orf53 | chromosome 12 open reading frame 53 | 196500 | 12 |
| 15 | C14orf82 | chromosome 14 open reading frame 82 | 145438 | 14 |
| 16 | C17orf107 | chromosome 17 open reading frame 107 | 100130311 | 17 |
| 17 | C18orf1 | chromosome 18 open reading frame 1 | 753 | 18 |
| 18 | C1orf95 | chromosome 1 open reading frame 95 | 375057 | 1 |
| 19 | C5orf42 | chromosome 5 open reading frame 42 | 65250 | 5 |
| 20 | CACNA1C | calcium channel, voltage-dependent, | 775 | 12 |
| | | L type, alpha 1C subunit | | |
| 21 | CAMK1D | calcium/calmodulin-dependent protein | 57118 | 10 |
| | | kinase ID | | |
| 22 | CAMTA1 | calmodulin binding transcription | 23261 | 1 |
| | | activator 1 | | |
| 23 | CBX6 | chromobox homolog 6 | 23466 | 22 |
| 24 | CCDC85A | coiled-coil domain containing 85A | 114800 | 2 |
| 25 | CCKBR | cholecystokinin B receptor | 887 | 11 |
| 26 | CD38 | CD38 molecule | 952 | 4 |
| 27 | CDKN2A | cyclin-dependent kinase inhibitor | 1029 | 9 |
| | | 2A (melanoma, p16, inhibits CDK4) | | |
| 28 | CH25H | cholesterol 25-hydroxylase | 9023 | 10 |
| 29 | CHST1 | carbohydrate (kcratan sulfate Gal-6) | 8534 | 11 |
| | | sulfotransferase 1 | | |
| 30 | CHST15 | carbohydrate (N-acetylgalactosamine | 51363 | 10 |
| | | 4-sulfate 6-O) sulfotransferase 15 | | |
| 31 | CNTLN | centlein, centrosomal protein | 54875 | 9 |
| 32 | CRHR1 | corticotropin releasing hormone | 1394 | 17 |
| | | receptor 1 | | |
| 33 | CRTC1 | CREB regulated transcription | 23373 | 19 |
| | | coactivator 1 | | |
| 34 | CXCR4 | chemokine (C-X-C motif) receptor 4 | 7852 | 2 |
| 35 | CYP1B1 | cytochrome P450, family 1, subfamily | 1545 | 2 |
| | | B, polypeptide 1 | | |
| 36 | DCTN2 | dynactin 2 (p50) | 10540 | 12 |
| 37 | DIDO1 | death inducer-obliterator 1 | 11083 | 20 |
| 38 | DMKN | dermokine | 93099 | 19 |
| 39 | DSE | dermatan sulfate epimerase | 29940 | 6 |
| 40 | DYNC1I1 | dynein, cytoplasmic 1, intermediate | 1780 | 7 |
| | | chain 1 | | |
| 41 | EML6 | echinoderm microtubule associated | 400954 | 2 |
| | | protein like 6 | | |
| 42 | ENOX1 | ecto-NOX disulfide-thiol exchanger 1 | 55068 | 13 |
| 43 | EPHA4_8030 | EPH receptor A4 | 2043 | 2 |
| 44 | EPHA4_6154 | EPH receptor A4 | 2043 | 2 |
| 45 | ESRRG | estrogen-related receptor gamma | 2104 | 1 |
| 46 | FAM176A | family with sequence similarity 176, | 84141 | 2 |
| | | member A | | |
| 47 | FAM78B | family with sequence similarity 78, | 149297 | 1 |
| | | member B | | |
| 48 | FBXO10 | F-box protein 10 | 26267 | 9 |
| 49 | FERMT2 | fermitin family member 2 | 10979 | 14 |
| 50 | FHOD3 | formin homology 2 domain containing 3 | 80206 | 18 |
| 51 | FLJ45079 | FLJ45079 protein | 400624 | 17 |
| 52 | FMNL1 | formin-like 1 | 752 | 17 |
| 53 | FOXP2 | forkhead box P2 | 93986 | 7 |
| 54 | FRMD4B | FERM domain containing 4B | 23150 | 3 |
| 55 | FZD8 | frizzled homolog 8 (Drosophila) | 8325 | 10 |
| 56 | GALNTL1 | UDP-N-acetyl-alpha-D- | 57452 | 14 |
| | | galactosamine:polypeptide N- | | |
| | | acetylgalactosaminyltransferase-like 1 | | |
| 57 | GALNTL4 | UDP-N-acetyl-alpha-D- | 374378 | 11 |
| | | galactosamine:polypeptide N- | | |
| | | acetylgalactosaminyltransferase-like 4 | | |
| 58 | GAS1 | growth arrest-specific 1 | 2619 | 9 |
| 59 | GBGT1 | globoside alpha-1,3-N- | 26301 | 9 |
| | | acetylgalactosaminyltransferase 1 | | |
| 60 | GLIPR2 | GLI pathogenesis-related 2 | 152007 | 9 |
| 61 | GNAI1 | guanine nucleotide binding protein (G | 2770 | 7 |
| | | protein), alpha inhibiting activity | | |
| | | polypeptide 1 | | |
| 62 | GNAL | guanine nucleotide binding protein (G | 2774 | 18 |
| | | protein), alpha activating activity | | |
| | | polypeptide, olfactory type | | |
| 63 | GPR37 | G protein-coupled receptor 37 | 2861 | 7 |
| | | (endothelin receptor type B-like) | | |
| 64 | GRASP | GRP1 (general receptor for | 160622 | 12 |
| | | phosphoinositides 1)-associated | | |
| | | scaffold protein | | |
| 65 | GRID1 | glutamate receptor, ionotropic, delta 1 | 2894 | 10 |
| 66 | GRM8 | glutamate receptor, metabotropic 8 | 2918 | 7 |
| 67 | GSC | goosecoid homeobox | 145258 | 14 |
| 68 | HAR1A | highly accelerated region 1A (non- | 768096 | 20 |
| | | protein coding) | | |
| 69 | HCN2 | hyperpolarization activated cyclic | 610 | 19 |
| | | nucleotide-gated potassium channel 2 | | |
| 70 | HEY2 | hairy/enhancer-of-split related with | 23493 | 6 |
| | | YRPW motif 2 | | |
| 71 | HIST1H2BE | histone cluster 1, H2be | 8344 | 6 |
| 72 | HS3ST3B1 | heparan sulfate (glucosamine) 3-O- | 9953 | 17 |
| | | sulfotransferase 3B1 | | |
| 73 | HTR7 | 5-hydroxytryptamine (serotonin) | 3363 | 10 |
| | | receptor 7 (adenylate cyclase-coupled) | | |
| 74 | IGFBP2 | insulin-like growth factor binding | 3485 | 2 |
| | | protein 2, 36 kDa | | |
| 75 | INHA | inhibin, alpha | 3623 | 2 |
| 76 | INSRR | insulin receptor-related receptor | 3645 | 1 |
| 77 | IRX3 | iroquois homeobox 3 | 79191 | 16 |
| 78 | ISM2 | isthmin 2 homolog (zebrafish) | 145501 | 14 |
| 79 | KCNG3 | potassium voltage-gated channel, | 170850 | 2 |
| | | subfamily G, member 3 | | |
| 80 | KCNK4 | potassium channel, subfamily K, member | 50801 | 11 |
| | | 4 | | |
| 81 | KCNS2 | potassium voltage-gated channel, delayed- | 3788 | 8 |
| | | rectifier, subfamily S, member 2 | | |
| 82 | KCTD15 | potassium channel tetramerisation domain | 79047 | 19 |
| | | containing 15 | | |
| 83 | KIAA1522 | KIAA1522 | 57648 | 1 |
| 84 | KIAA1614 | KIAA1614 | 57710 | 1 |
| 85 | KIF26A | kinesin family member 26A | 26153 | 14 |
| 86 | KL | klotho | 9365 | 13 |
| 87 | KLF15 | Kruppel-like factor 15 | 28999 | 3 |
| 88 | KLHL10 | kelch-like 10 (Drosophila) | 317719 | 17 |
| 89 | KRT77 | keratin 77 | 374454 | 12 |
| 90 | KSR2 | kinase suppressor of ras 2 | 283455 | 12 |
| 91 | LBH | limb bud and heart development homolog | 81606 | 2 |
| | | (mouse) | | |
| 92 | LMX1A | LIM homeobox transcription factor 1, | 4009 | 1 |
| | | alpha | | |
| 93 | LMX1B | LIM homeobox transcription factor 1, beta | 4010 | 9 |
| 94 | LOC100526820 | hypothetical LOC100526820 | 100526820 | 6 |
| 95 | LONRF2 | LON peptidase N-terminal domain and | 164832 | 2 |
| | | ring finger 2 | | |
| 96 | LRFN2 | leucine rich repeat and fibronectin type | 57497 | 6 |
| | | III domain containing 2 | | |
| 97 | LRRN1 | leucine rich repeat neuronal 1 | 57633 | 3 |
| 98 | MAF | v-maf musculoaponeurotic fibrosarcoma | 4094 | 16 |
| | | oncogene homolog (avian) | | |
| 99 | MAFB | v-maf musculoaponeurotic fibrosarcoma | 9935 | 20 |
| | | oncogene homolog B (avian) | | |
| 100 | MARK1 | MAP/microtubule affinity-regulating | 4139 | 1 |
| | | kinase 1 | | |
| 101 | ADAMTSL4-AS1 | ADAMTSL4 antisense RNA 1 | 574406 | 1 |
| 102 | PGBD5 | piggyBac transposable element derived 5 | 79605 | 1 |
| 103 | HSPA12A | heat shock protein 12A | 259217 | 10 |
| 104 | SFTPD | surfactant protein D | ENSG00000133661 | 10 |
| 105 | LOC107984507 | hypothetical LOC107984507 | 107984507 | 12 |
| 106 | LOC100128253 | hypothetical LOC100128253 | 100128253 | 12 |
| 107 | CISTR | chondrogenesis-associated transcript | 102216268 | 12 |
| 108 | SLC16A7 | solute carrier family 16 member 7 | 9194 | 12 |
| 109 | CTXND1 | cortexin domain containing 1 | 100996492 | 15 |
| 110 | MAX.chr15.4912 | — | SEQ ID NO: 1 | 15 |
| 111 | ZNF423 | zinc finger protein 423 | 23090 | 16 |
| 112 | RBFOX1 | RNA binding fox-1 homolog 1 | ENSG00000078328 | 16 |
| 113 | LOC105376772 | hypothetical LOC105376772 | 105376772 | 16 |
| 114 | GSE1 | Gsel coiled-coil protein | ENSG00000131149 | 16 |
| 115 | MAX.chr17.8070 | — | SEQ ID NO: 2 | 17 |
| 116 | ZNF709_6125 | zinc finger protein 709 | 163051 | 19 |
| 117 | MAX.chr19.3439 | — | SEQ ID NO: 3 | 19 |
| 118 | GREB1 | growth regulating estrogen receptor | ENSG00000196208 | 2 |
| | | binding 1 | | |
| 119 | CYP27C1 | cytochrome P450 family 27 subfamily C | 339761 | 2 |
| | | member 1 | | |
| 120 | AC068134.6 | — | ENSG00000237087 | 2 |
| 121 | LOC388780 | hypothetical LOC388780 | 388780 | 20 |
| 122 | STOX2_8441 | Storkhead Box 2 | 56977 | 4 |
| 123 | PPARGC1A | PPARG coactivator 1 alpha | 10891 | 4 |
| 124 | MAX.chr4.4552 | — | SEQ ID NO: 4 | 4 |
| 125 | PDZD2 | PDZ domain containing 2 | 23037 | 5 |
| 126 | MAX.chr6.6743 | — | SEQ ID NO: 5 | 6 |
| 127 | LPAL2 | lipoprotein(a) like 2 (pseudogene) | ENSG00000290613 | 6 |
| 128 | CDK 14 | Cyclin Dependent Kinase 14 | ENSG00000058091 | 7 |
| 129 | MAX.chr8.3003 | — | SEQ ID NO: 6 | 8 |
| 130 | MCOLN2 | mucolipin 2 | 255231 | 1 |
| 131 | MEGF11 | multiple EGF-like-domains 11 | 84465 | 15 |
| 132 | MFSD11 | major facilitator superfamily domain | 79157 | 17 |
| | | containing 11 | | |
| 133 | MRC2 | mannose receptor, C type 2 | 9902 | 17 |
| 134 | MSX1 | msh homeobox 1 | 4487 | 4 |
| 135 | NAT8L | N-acetyltransferase 8-like (GCN5-related, | 339983 | 4 |
| | | putative) | | |
| 136 | NAV1 | neuron navigator 1 | 89796 | 1 |
| 137 | NBEA | neurobeachin | 26960 | 13 |
| 138 | NCRNA00092 | non-protein coding RNA 92 | 100188953 | 9 |
| 139 | NECAB2 | N-terminal EF-hand calcium binding | 54550 | 16 |
| | | protein 2 | | |
| 140 | NEURL | neuralized homolog (Drosophila) | 9148 | 10 |
| 141 | NGEF | neuronal guanine nucleotide exchange | 25791 | 2 |
| | | factor | | |
| 142 | NHLH2 | nescient helix loop helix 2 | 4808 | 1 |
| 143 | NKD1 | naked cuticle homolog 1 (Drosophila) | 85407 | 16 |
| 144 | NLGN1 | neuroligin 1 | 22871 | 3 |
| 145 | NOG | noggin | 9241 | 17 |
| 146 | NPPC | natriuretic peptide precursor C | 4880 | 2 |
| 147 | NR3C1 | nuclear receptor subfamily 3, group C, | 2908 | 5 |
| | | member 1 (glucocorticoid receptor) | | |
| 148 | NRXN2 | neurexin 2 | 9379 | 11 |
| 149 | NTN1 | netrin 1 | 9423 | 17 |
| 150 | OBSCN | obscurin, cytoskeletal calmodulin and | 84033 | 1 |
| | | titin-interacting RhoGEF | | |
| 151 | OCA2 | oculocutaneous albinism II | 4948 | 15 |
| 152 | OSBPL1A | oxysterol binding protein-like 1A | 114876 | 18 |
| 153 | OXR1 | oxidation resistance 1 | 55074 | 8 |
| 154 | P2RY1 | purinergic receptor P2Y, G-protein | 5028 | 3 |
| | | coupled, 1 | | |
| 155 | PDE2A | phosphodiesterase 2A, cGMP-stimulated | 5138 | 11 |
| 156 | PDE9A | phosphodiesterase 9A | 5152 | 21 |
| 157 | PDGFRA_3555 | platelet-derived growth factor receptor, | 5156 | 4 |
| | | alpha polypeptide | | |
| 158 | PDGFRA_5942 | platelet-derived growth factor receptor, | 5156 | 4 |
| | | alpha polypeptide | | |
| 159 | PDGFRA_9128 | platelet-derived growth factor receptor, | 5156 | 4 |
| | | alpha polypeptide | | |
| 160 | PID1 | phosphotyrosine interaction domain | 55022 | 2 |
| | | containing 1 | | |
| 161 | PLCG2 | phospholipase C, gamma 2 | 5336 | 16 |
| | | (phosphatidylinositol-specific) | | |
| 162 | PLCL1 | phospholipase C-like 1 | 5334 | 2 |
| 163 | PNPLA3 | patatin-like phospholipase domain | 80339 | 22 |
| | | containing 3 | | |
| 164 | POU3F1 | POU class 3 homeobox 1 | 5453 | 1 |
| 165 | POU3F2 | POU class 3 homeobox 2 | 5454 | 6 |
| 166 | PRKACB | protein kinase, cAMP-dependent, catalytic, | 5567 | 1 |
| | | beta | | |
| 167 | PRKAR2B | protein kinase, cAMP-dependent, | 5577 | 7 |
| | | regulatory, type II, beta | | |
| 168 | PRKG1 | protein kinase, cGMP-dependent, type I | 5592 | 10 |
| 169 | PRR18 | proline rich 18 | 285800 | 6 |
| 170 | PRR5L | proline rich 5 like | 79899 | 11 |
| 171 | PTHLH | parathyroid hormone-like hormone | 5744 | 12 |
| 172 | PYGL | phosphory lase, glycogen, liver | 5836 | 14 |
| 173 | RARG | retinoic acid receptor, gamma | 5916 | 12 |
| 174 | RHBDL3 | rhomboid, veinlet-like 3 (Drosophila) | 162494 | 17 |
| 175 | RIMS2 | regulating synaptic membrane exocytosis 2 | 9699 | 8 |
| 176 | ROR2 | receptor tyrosine kinase-like orphan | 4920 | 9 |
| | | receptor 2 | | |
| 177 | RPRML | reprimo-like | 388394 | 17 |
| 178 | SDK2 | sidekick homolog 2 (chicken) | 54549 | 17 |
| 179 | SOX9 | SRY (sex determining region Y)-box 9 | 6662 | 17 |
| 180 | STOX2_6730 | storkhead box 2 | 56977 | 4 |
| 181 | SYNGR1 | synaptogyrin 1 | 9145 | 22 |
| 182 | TAC4 | tachykinin 4 (hemokinin) | 255061 | 17 |
| 183 | TNFRSF19 | tumor necrosis factor receptor | 55504 | 13 |
| | | superfamily, member 19 | | |
| 184 | TRANK1 | tetratricopeptide repeat and ankyrin | 9881 | 3 |
| | | repeat containing 1 | | |
| 185 | TSPAN33 | tetraspanin 33 | 340348 | 7 |
| 186 | TSPAN4 | tetraspanin 4 | 7106 | 11 |
| 187 | TSPAN5 | tetraspanin 5 | 10098 | 4 |
| 188 | UBE2E2 | ubiquitin-conjugating enzyme E2E 2 | 7325 | 3 |
| | | (UBC4/5 homolog, yeast) | | |
| 189 | UCHL1 | ubiquitin carboxyl-terminal esterase L1 | 7345 | 4 |
| | | (ubiquitin thiolesterase) | | |
| 190 | UNC5A | unc-5 homolog A (C. elegans) | 90249 | 5 |
| 191 | VASH2 | vasohibin 2 | 79805 | 1 |
| 192 | VIM | vimentin | 7431 | 10 |
| 193 | WNT6 | wingless-type MMTV integration site | 7475 | 2 |
| | | family, member 6 | | |
| 194 | ZBTB10 | zinc finger and BTB domain containing 10 | 65986 | 8 |
| 195 | ZNF680 | zinc finger protein 680 | 340252 | 7 |
| 196 | ZNF709_4918 | zinc finger protein 709 | 163051 | 19 |
| 197 | ZNF738 | zinc finger protein 738 | 148203 | 19 |
| 198 | ZNF808 | zinc finger protein 808 | 388558 | 19 |
| 199 | ZNF845 | zinc finger protein 845 | 91664 | 19 |

In Table 2, DMR performance characteristics from the whole genome sequencing (WGS) study are provided. Additionally, 156 DRs were also identified in the separate reduced representation bisulfite sequencing (RRBS) study using independent tissue biopsies, thus validating the results of the WGS study.

TABLE 2
Performance characteristics (AUC and fold-change) of DMRs capable of discriminating
Barrett's Esophagus (BE) and/or normal esophageal samples from precancerous
high grade dysplasia (HGD) and/or esophageal adenocarcinoma.

| DMR | Gene | | | | Identification |
|---|---|---|---|---|---|
| No. | Annotation | AUC | FC | P value | Method |
| 1 | ACVRL1 | 0.8287 | 7.04 | 0.002219 | WGS and RRBS |
| 2 | ADAMTS8 | 0.821 | 5.271 | 0.01576 | WGS and RRBS |
| 3 | ADAP2 | 0.7207 | 6.157 | 0.01985 | WGS and RRBS |
| 4 | ADRBK2 | 0.7963 | 7.635 | 0.007314 | WGS and RRBS |
| 5 | AKR1B1 | 0.7377 | 4.686 | 0.08372 | WGS and RRBS |
| 6 | ANK1 | 0.7299 | 19.99 | 0.1993 | WGS and RRBS |
| 7 | ANKRD13B | 0.7847 | 5.949 | 0.0194 | WGS and RRBS |
| 8 | ANXA6 | 0.7315 | 6.239 | 0.04294 | WGS and RRBS |
| 9 | ARNT2 | 0.7525 | 10.61 | 0.09539 | WGS and RRBS |
| 10 | B4GALNT2 | 0.8225 | 6.747 | 0.001132 | WGS and RRBS |
| 11 | BACH2 | 0.7963 | 7.393 | 0.01527 | WGS and RRBS |
| 12 | BCL11A | 0.7261 | 19.5 | 0.1107 | WGS and RRBS |
| 13 | BSCL2 | 0.7168 | 116 | 0.3048 | WGS and RRBS |
| 14 | C12orf53 | 0.75 | 12.57 | 0.03578 | WGS |
| 15 | C14orf82 | 0.7029 | 16.13 | 0.159 | WGS and RRBS |
| 16 | C17orf107 | 0.6227 | 6.113 | 0.03399 | WGS |
| 17 | C18orf1 | 0.7292 | 9.244 | 0.05688 | WGS and RRBS |
| 18 | C1orf95 | 0.7407 | 3.115 | 0.04963 | WGS and RRBS |
| 19 | C5orf42 | 0.767 | 8.671 | 0.04022 | WGS and RRBS |
| 20 | CACNA1C | 0.7407 | 18.41 | 0.03286 | WGS |
| 21 | CAMK1D | 0.6914 | 3.071 | 0.03296 | WGS and RRBS |
| 22 | CAMTA1 | 0.8272 | 24.99 | 0.1039 | WGS and RRBS |
| 23 | CBX6 | 0.7531 | 7.864 | 0.02758 | WGS |
| 24 | CCDC85A | 0.7515 | 5.42 | 0.01691 | WGS and RRBS |
| 25 | CCKBR | 0.804 | 6.057 | 0.01132 | WGS |
| 26 | CD38 | 0.7407 | 8.125 | 0.00571 | WGS and RRBS |
| 27 | CDKN2A | 0.8287 | 15.83 | 0.002328 | WGS and RRBS |
| 28 | CH25H | 0.7716 | 5.081 | 0.002563 | WGS |
| 29 | CHST1 | 0.8441 | 6.22 | 0.00402 | WGS and RRBS |
| 30 | CHST15 | 0.7446 | 8.939 | 0.1162 | WGS and RRBS |
| 31 | CNTLN | 0.8611 | 8.667 | 0.003266 | WGS |
| 32 | CRHR1 | 0.8063 | 7.34 | 0.00645 | WGS and RRBS |
| 33 | CRTC1 | 0.777 | 22.86 | 0.1381 | WGS |
| 34 | CXCR4 | 0.6597 | 32.18 | 0.2377 | WGS |
| 35 | CYP1B1 | 0.787 | 6.307 | 0.002258 | WGS and RRBS |
| 36 | DCTN2 | 0.7246 | 6.358 | 0.05421 | WGS |
| 37 | DIDO1 | 0.7793 | 9.404 | 0.01428 | WGS and RRBS |
| 38 | DMKN | 0.8117 | 25.21 | 0.03412 | WGS |
| 39 | DSE | 0.6991 | 12.63 | 0.1568 | WGS and RRBS |
| 40 | DYNC1I1 | 0.7307 | 6.937 | 0.02997 | WGS and RRBS |
| 41 | EML6 | 0.7114 | 12.33 | 0.07832 | WGS and RRBS |
| 42 | ENOX1 | 0.8194 | 8.668 | 0.00108 | WGS and RRBS |
| 43 | EPHA4_8030 | 0.7446 | 17.78 | 0.1322 | WGS |
| 44 | EPHA4_6154 | 0.8063 | 10.79 | 0.03819 | WGS and RRBS |
| 45 | ESRRG | 0.8503 | 4.314 | 0.04048 | WGS |
| 46 | FAM176A | 0.7994 | 6.394 | 0.04177 | WGS and RRBS |
| 47 | FAM78B | 0.6552 | 7.213 | 0.06662 | WGS |
| 48 | FBXO10 | 0.7215 | 10.45 | 0.05047 | WGS and RRBS |
| 49 | FERMT2 | 0.8063 | 5.575 | 0.003481 | WGS and RRBS |
| 50 | FHOD3 | 0.8403 | 14.72 | 0.01122 | WGS and RRBS |
| 51 | FLJ45079 | 0.6744 | 7.054 | 0.06199 | WGS and RRBS |
| 52 | FMNL1 | 0.7415 | 34.42 | 0.1368 | WGS and RRBS |
| 53 | FOXP2 | 0.8596 | 8.099 | 0.004965 | WGS |
| 54 | FRMD4B | 0.7346 | 9.195 | 0.007747 | WGS and RRBS |
| 55 | FZD8 | 0.733 | 10.1 | 0.04281 | WGS and RRBS |
| 56 | GALNTL1 | 0.7654 | 5.151 | 0.01165 | WGS and RRBS |
| 57 | GALNTL4 | 0.8056 | 4.857 | 0.006806 | WGS and RRBS |
| 58 | GAS1 | 0.706 | 9.754 | 0.0796 | WGS and RRBS |
| 59 | GBGT1 | 0.75 | 6.037 | 0.006868 | WGS and RRBS |
| 60 | GLIPR2 | 0.6667 | 97910000 | 0.995 | WGS and RRBS |
| 61 | GNAI1 | 0.7577 | 8.411 | 0.0121 | WGS and RRBS |
| 62 | GNAL | 0.7639 | 4.168 | 0.03524 | WGS and RRBS |
| 63 | GPR37 | 0.6235 | 10.46 | 0.07179 | WGS |
| 64 | GRASP | 0.8333 | 8.254 | 0.001828 | WGS and RRBS |
| 65 | GRID1 | 0.8056 | 7.666 | 0.001441 | WGS and RRBS |
| 66 | GRM8 | 0.8302 | 5.385 | 0.009953 | WGS and RRBS |
| 67 | GSC | 0.8009 | 6.229 | 0.005581 | WGS and RRBS |
| 68 | HAR1A | 0.7384 | 4.553 | 0.05431 | WGS and RRBS |
| 69 | HCN2 | 0.8056 | 6.387 | 0.01634 | WGS and RRBS |
| 70 | HEY2 | 0.7346 | 9.638 | 0.05959 | WGS and RRBS |
| 71 | HIST1H2BE | 0.7716 | 32.13 | 0.04715 | WGS and RRBS |
| 72 | HS3ST3B1 | 0.6404 | 9.757 | 0.0191 | WGS and RRBS |
| 73 | HTR7 | 0.7562 | 7.269 | 0.04768 | WGS |
| 74 | IGFBP2 | 0.7114 | 9.034 | 0.0939 | WGS and RRBS |
| 75 | INHA | 0.8426 | 7.12 | 0.001955 | WGS and RRBS |
| 76 | INSRR | 0.7384 | 3.735 | 0.04924 | WGS |
| 77 | IRX3 | 0.7546 | 10.26 | 0.03043 | WGS and RRBS |
| 78 | ISM2 | 0.7191 | 4.958 | 0.1061 | WGS and RRBS |
| 79 | KCNG3 | 0.8395 | 23.25 | 0.01075 | WGS and RRBS |
| 80 | KCNK4 | 0.7639 | 4.528 | 0.03311 | WGS and RRBS |
| 81 | KCNS2 | 0.7984 | 9.082 | 0.002112 | WGS and RRBS |
| 82 | KCTD15 | 0.7137 | 8.901 | 0.01359 | WGS and RRBS |
| 83 | KIAA1522 | 0.8071 | 6.611 | 0.01875 | WGS |
| 84 | KIAA1614 | 0.7554 | 5.669 | 0.03151 | WGS and RRBS |
| 85 | KIF26A | 0.8627 | 6.51 | 0.001629 | WGS |
| 86 | KL | 0.8167 | 14.04 | 0.001855 | WGS and RRBS |
| 87 | KLF15 | 0.8009 | 20.1 | 0.1942 | WGS |
| 88 | KLHL10 | 0.7269 | 14.13 | 0.2248 | WGS and RRBS |
| 89 | KRT77 | 0.6968 | 6.079 | 0.02858 | WGS |
| 90 | KSR2 | 0.6698 | 36.66 | 0.1328 | WGS and RRBS |
| 91 | LBH | 0.7083 | 5.019 | 0.02499 | WGS and RRBS |
| 92 | IMX1A | 0.8349 | 6.408 | 0.001447 | WGS and RRBS |
| 93 | LMX1B | 0.8812 | 7.868 | 0.000888 | WGS and RRBS |
| 94 | LOC100526820 | 0.7292 | 3.786 | 0.04844 | WGS and RRBS |
| 95 | LONRF2 | 0.7832 | 7.785 | 0.04936 | WGS and RRBS |
| 96 | LRFN2 | 0.7531 | 35.62 | 0.206 | WGS and RRBS |
| 97 | LRRN1 | 0.6744 | 5.213 | 0.01324 | WGS |
| 98 | MAF | 0.6944 | 5.611 | 0.06278 | WGS |
| 99 | MAFB | 0.9306 | 28.11 | 0.01776 | WGS and RRBS |
| 100 | MARK1 | 0.7762 | 4.404 | 0.04193 | WGS and RRBS |
| 101 | ADAMTSL4-AS1 | 0.8079 | 5.891 | 0.00317 | WGS |
| 102 | PGBD5 | 0.7176 | 7.344 | 0.04185 | WGS and RRBS |
| 103 | IISPA12A | 0.7654 | 6.691 | 0.005638 | WGS and RRBS |
| 104 | SFTPD | 0.7747 | 21.33 | 0.03021 | WGS and RRBS |
| 105 | LOC107984507 | 0.7377 | 15.88 | 0.04043 | WGS and RRBS |
| 106 | LOC100128253 | 0.8086 | 12.75 | 0.0254 | WGS and RRBS |
| 107 | CISTR | 0.6813 | 6.176 | 0.02384 | WGS and RRBS |
| 108 | SLC16A7 | 0.6397 | 5.315 | 0.03028 | WGS and RRBS |
| 109 | CTXND1 | 0.8225 | 5.394 | 0.01201 | WGS and RRBS |
| 110 | MAX.chr15.4912 | 0.8056 | 13.53 | 0.008849 | WGS |
| 111 | ZNF423 | 0.7114 | 5.17 | 0.03415 | WGS and RRBS |
| 112 | RBFOX1 | 0.7307 | 5.041 | 0.03037 | WGS |
| 113 | LOC105376772 | 0.692 | 7.421 | 0.01115 | WGS and RRBS |
| 114 | GSE1 | 0.7022 | 5.724 | 0.021 | WGS and RRBS |
| 115 | MAX.chr17.8070 | 0.8056 | 9.185 | 0.07304 | WGS |
| 116 | ZNF709_6125 | 0.6813 | 5.778 | 0.1457 | WGS |
| 117 | MAX.chr19.3439 | 0.7616 | 15.05 | 0.08682 | WGS and RRBS |
| 118 | GREB1 | 0.7215 | 6.75 | 0.02349 | WGS and RRBS |
| 119 | CYP27C1 | 0.7068 | 3.296 | 0.04444 | WGS and RRBS |
| 120 | AC068134.6 | 0.6871 | 8.009 | 0.0934 | WGS and RRBS |
| 121 | LOC388780 | 0.6921 | 39.14 | 0.1991 | WGS and RRBS |
| 122 | STOX2_8441 | 0.7523 | 34.09 | 0.13 | WGS and RRBS |
| 123 | PPARGC1A | 0.7222 | 3.573 | 0.02185 | WGS and RRBS |
| 124 | MAX.chr4.4552 | 0.7677 | 12.97 | 0.04582 | WGS |
| 125 | PDZD2 | 0.7654 | 11.6 | 0.02463 | WGS and RRBS |
| 126 | MAX.chr6.6743 | 0.7337 | 10.93 | 0.06234 | WGS and RRBS |
| 127 | LPAL2 | 0.6065 | 20.66 | 0.2297 | WGS and RRBS |
| 128 | CDK14 | 0.6551 | 12.87 | 0.05557 | WGS and RRBS |
| 129 | MAX.chr8.3003 | 0.7801 | 4.706 | 0.01352 | WGS |
| 130 | MCOLN2 | 0.8171 | 6.798 | 0.01497 | WGS and RRBS |
| 131 | MEGF11 | 0.8086 | 8.369 | 0.009733 | WGS and RRBS |
| 132 | MFSD11 | 0.8025 | 7.374 | 0.05609 | WGS |
| 133 | MRC2 | 0.7438 | 33.4 | 0.1286 | WGS and RRBS |
| 134 | MSX1 | 0.8133 | 18.68 | 0.101 | WGS and RRBS |
| 135 | NAT8L | 0.6921 | 44.65 | 0.1631 | WGS and RRBS |
| 136 | NAV1 | 0.8403 | 7.671 | 0.002609 | WGS and RRBS |
| 137 | NBEA | 0.7315 | 4.685 | 0.01885 | WGS and RRBS |
| 138 | NCRNA00092 | 0.7284 | 3.91 | 0.02402 | WGS and RRBS |
| 139 | NECAB2 | 0.7845 | 8.305 | 0.01135 | WGS |
| 140 | NEURL | 0.7755 | 5.794 | 0.009554 | WGS and RRBS |
| 141 | NGEF | 0.7608 | 16.58 | 0.138 | WGS and RRBS |
| 142 | NHLH2 | 0.8179 | 11.2 | 0.01127 | WGS and RRBS |
| 143 | NKD1 | 0.6111 | 49.59 | 0.323 | WGS and RRBS |
| 144 | NLGN1 | 0.8406 | 7.439 | 0.009903 | WGS and RRBS |
| 145 | NOG | 0.7191 | 10 | 0.0161 | WGS and RRBS |
| 146 | NPPC | 0.7917 | 5.54 | 0.01113 | WGS and RRBS |
| 147 | NR3C1 | 0.7315 | 6.953 | 0.04844 | WGS and RRBS |
| 148 | NRXN2 | 0.7681 | 14.14 | 0.03384 | WGS and RRBS |
| 149 | NTN1 | 0.6435 | 5.516 | 0.05691 | WGS |
| 150 | OBSCN | 0.7855 | 8.194 | 0.01644 | WGS and RRBS |
| 151 | OCA2 | 0.7346 | 5.178 | 0.009414 | WGS and RRBS |
| 152 | OSBPL1A | 0.6798 | 5.13 | 0.1036 | WGS and RRBS |
| 153 | OXR1 | 0.7006 | 9.448 | 0.04784 | WGS and RRBS |
| 154 | P2RY1 | 0.7407 | 12.65 | 0.06068 | WGS and RRBS |
| 155 | PDE2A | 0.6613 | 6.054 | 0.09965 | WGS and RRBS |
| 156 | PDE9A | 0.6397 | 9.226 | 0.07582 | WGS |
| 157 | PDGFRA_3555 | 0.7407 | 3.065 | 0.02858 | WGS |
| 158 | PDGFRA_5942 | 0.8102 | 5.404 | 0.02282 | WGS |
| 159 | PDGFRA_9128 | 0.7747 | 14.64 | 0.03034 | WGS and RRBS |
| 160 | PID1 | 0.8318 | 11.3 | 0.05873 | WGS and RRBS |
| 161 | PLCG2 | 0.7222 | 4.086 | 0.02395 | WGS and RRBS |
| 162 | PLCL1 | 0.804 | 4.322 | 0.06646 | WGS and RRBS |
| 163 | PNPLA3 | 0.7014 | 8.44 | 0.01044 | WGS and RRBS |
| 164 | POU3F1 | 0.8202 | 7.979 | 0.007761 | WGS and RRBS |
| 165 | POU3F2 | 0.607 | 4.37 | 0.05324 | WGS and RRBS |
| 166 | PRKACB | 0.7377 | 5.762 | 0.04194 | WGS and RRBS |
| 167 | PRKAR2B | 0.7917 | 6.517 | 0.01218 | WGS and RRBS |
| 168 | PRKG1 | 0.8565 | 8.025 | 0.000766 | WGS and RRBS |
| 169 | PRR18 | 0.8025 | 5.382 | 0.003298 | WGS and RRBS |
| 170 | PRR5L | 0.7793 | 33.53 | 0.07633 | WGS and RRBS |
| 171 | PTHLH | 0.7978 | 23.26 | 0.09052 | WGS and RRBS |
| 172 | PYGL | 0.8009 | 3.879 | 0.00091 | WGS and RRBS |
| 173 | RARG | 0.8143 | 10.89 | 0.002373 | WGS |
| 174 | RHBDL3 | 0.7924 | 16.98 | 0.02684 | WGS and RRBS |
| 175 | RIMS2 | 0.8086 | 6.884 | 0.001134 | WGS and RRBS |
| 176 | ROR2 | 0.8009 | 4.83 | 0.000729 | WGS and RRBS |
| 177 | RPRML | 0.7963 | 11.33 | 0.03475 | WGS and RRBS |
| 178 | SDK2 | 0.7809 | 14.26 | 0.06459 | WGS and RRBS |
| 179 | SOX9 | 0.8719 | 6.713 | 0.000413 | WGS and RRBS |
| 180 | STOX2_6730 | 0.8534 | 6.04 | 0.000143 | WGS and RRBS |
| 181 | SYNGR1 | 0.7662 | 7.99 | 0.005426 | WGS and RRBS |
| 182 | TAC4 | 0.7685 | 6.92 | 0.05388 | WGS and RRBS |
| 183 | TNFRSF19 | 0.7083 | 11.16 | 0.06492 | WGS |
| 184 | TRANK1 | 0.75 | 7.474 | 0.01273 | WGS and RRBS |
| 185 | TSPAN33 | 0.7724 | 7.268 | 0.02252 | WGS |
| 186 | TSPAN4 | 0.8105 | 5.189 | 0.005198 | WGS and RRBS |
| 187 | TSPAN5 | 0.8395 | 7.777 | 0.01024 | WGS |
| 188 | UBE2E2 | 0.7153 | 11.73 | 0.0118 | WGS and RRBS |
| 189 | UCHL1 | 0.7793 | 6.55 | 0.005504 | WGS and RRBS |
| 190 | UNC5A | 0.7647 | 4.793 | 0.009149 | WGS and RRBS |
| 191 | VASH2 | 0.7377 | 5.452 | 0.02439 | WGS and RRBS |
| 192 | VIM | 0.7299 | 5.984 | 0.03557 | WGS |
| 193 | WNT6 | 0.7207 | 5.76 | 0.01222 | WGS |
| 194 | ZBTB10 | 0.7508 | 18.75 | 0.05552 | WGS and RRBS |
| 195 | ZNF680 | 0.6042 | 13.72 | 0.2049 | WGS and RRBS |
| 196 | ZNF709_4918 | 0.7052 | 13.96 | 0.06003 | WGS and RRBS |
| 197 | ZNF738 | 0.6458 | 3.894 | 0.07532 | WGS and RRBS |
| 198 | ZNF808 | 0.6389 | 5.045 | 0.07821 | WGS and RRBS |
| 199 | ZNF845 | 0.7153 | 8.821 | 0.02854 | WGS and RRBS |

Table 3 highlights the top DMRs with AUCs>0.75, and FC>5 in both independent WGS and RRBS datasets. The DMRs in Table 3 represent a subset of DMRs that demonstrated the highest performance in both studies.

TABLE 3
Performance characteristics (AUC and fold-change) of highest
performing DMRs capable of discriminating Barrett's Esophagus
(BE) and/or normal esophageal samples from precancerous high
grade dysplasia (HGD) and/or esophageal adenocarcinoma.

| Gene | AUC | FC | AUC | FC |
|---|---|---|---|---|
| Annotation | (Brushing) | (Brushing) | (Tissue) | (Tissue) |
| BACH2 | 0.7963 | 7.393 | 0.7682 | 12.21 |
| C5orf42 | 0.767 | 8.671 | 0.7604 | 6.469 |
| FHOD3 | 0.8403 | 14.72 | 0.7526 | 8.868 |
| HIST1H2BE | 0.7716 | 32.13 | 0.7578 | 8.577 |
| IRX3 | 0.7546 | 10.26 | 0.7812 | 7.428 |
| KIAA1614 | 0.7554 | 5.669 | 0.7917 | 7.187 |
| LONRF2 | 0.7832 | 7.785 | 0.7956 | 5.7 |
| MAFB | 0.9306 | 28.11 | 0.8359 | 55.45 |
| PDGFRA_9128 | 0.7747 | 14.64 | 0.8229 | 6.588 |
| PID1 | 0.8318 | 11.3 | 0.862 | 5.101 |
| POU3F1 | 0.7801 | 13.24 | 0.7865 | 11.7 |
| PRR5L | 0.7793 | 33.53 | 0.7969 | 9.496 |
| RHBDL3 | 0.7924 | 16.98 | 0.776 | 5.366 |
| SDK2 | 0.7809 | 14.26 | 0.8802 | 8.905 |

Taken together, the merged data is a validation of the 156 DMRs. The two sequencing studies employed independent sample sets that were also different in type. The RRBS data were from clinical tissue biopsies while the WGS data were endoscopic esophageal brushings. Yet common genes were found in both, and not simply widely disparate regions of these genes, but in many cases overlapping sequence fragments. And with similar performance characteristics.

### Example 2

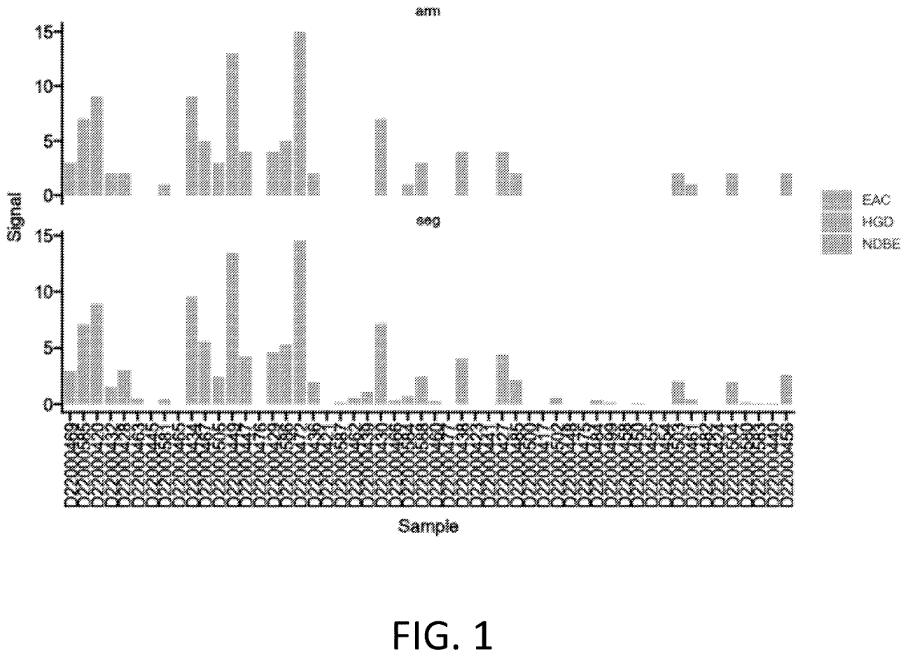

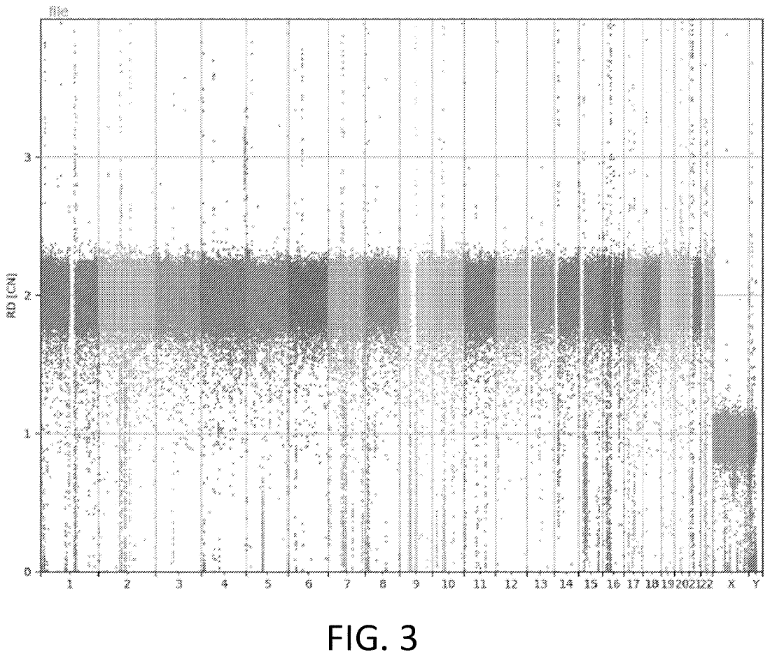

Experiments were also conducted to assess copy number variation (CNV), including polyploidy and aneuploidy determinations, for discriminating Barrett's Esophagus (BE) and/or normal esophageal samples from precancerous high grade dysplasia (HGD) and/or esophageal adenocarcinoma. In particular, another important aspect of the present disclosure is the ability to utilize sequencing reads from a cytosine converted genome to make copy number aberration (CNA) determinations, including polyploidy and aneuploidy. Most technologies use direct NGS on wild-type, unconverted DNA. Here, methylation and CNV read outs were obtained simultaneously from the same chemistry/dataset. For the WGS brushing study, CNVkit and AneuploidyScore software were used to make aneuploidy calls and generate numerical scores with the NE cohort as the reference. These normal esophageal (NE) samples are defined as euploid (2 copies of the 22 somatic chromosomes) and were confirmed with chromosomal self-reference scatter plots (see, e.g., FIG. 3). The mapped deduplicated reads were used for the analysis, which were apriori corrected for repetitive sequences, GC content, and PCR duplicates. The calculated aneuploidy score (AS) for each sample is the total number of chromosomal arm-level gains and losses, adjusted for ploidy. See, e.g., FIG. 1 for the EAC, HGD, and NDBE sample arm-level and segmented aneuploidy scores (y-axis, signal).

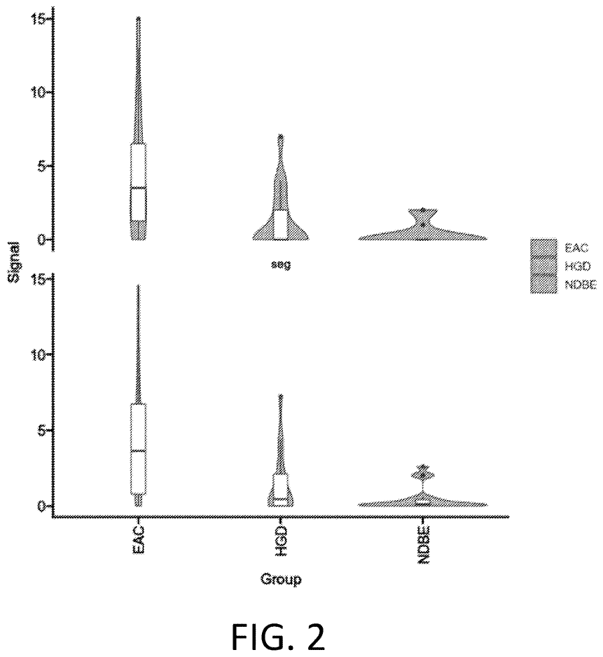

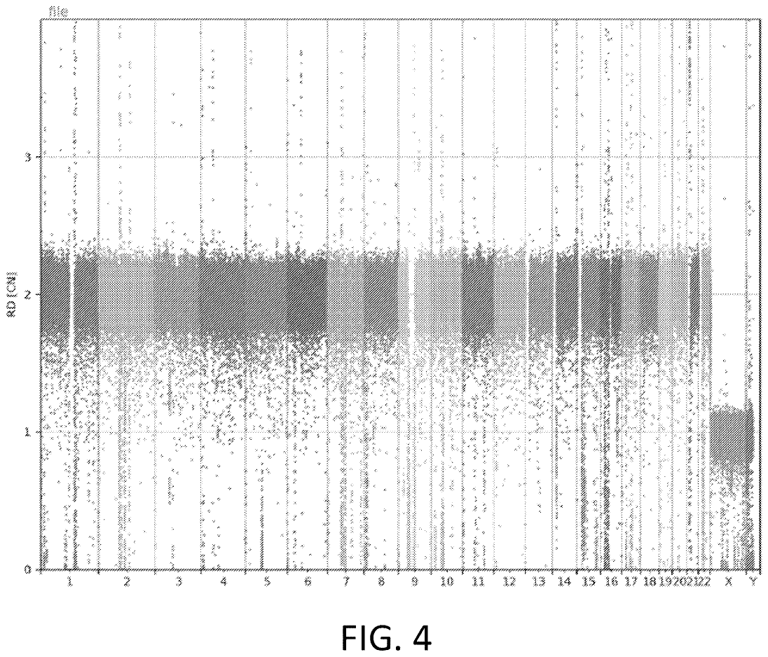

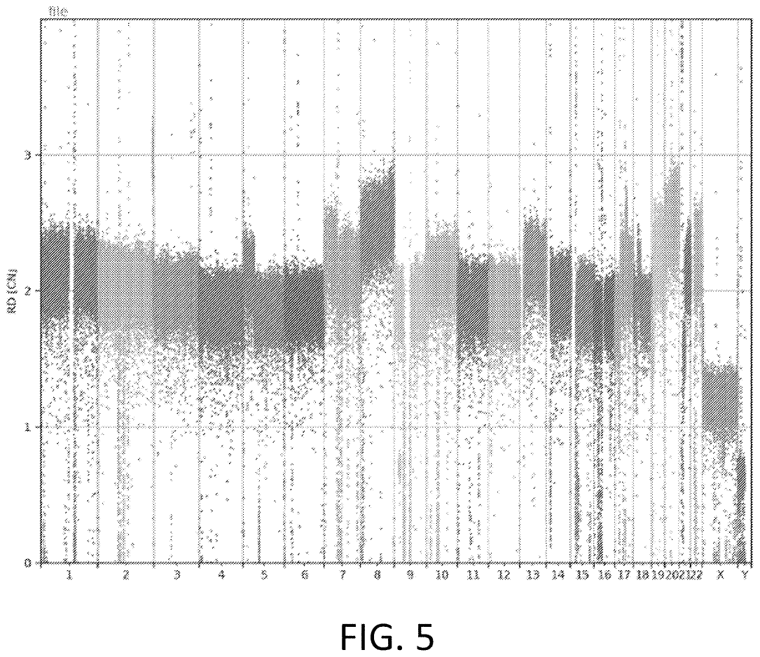

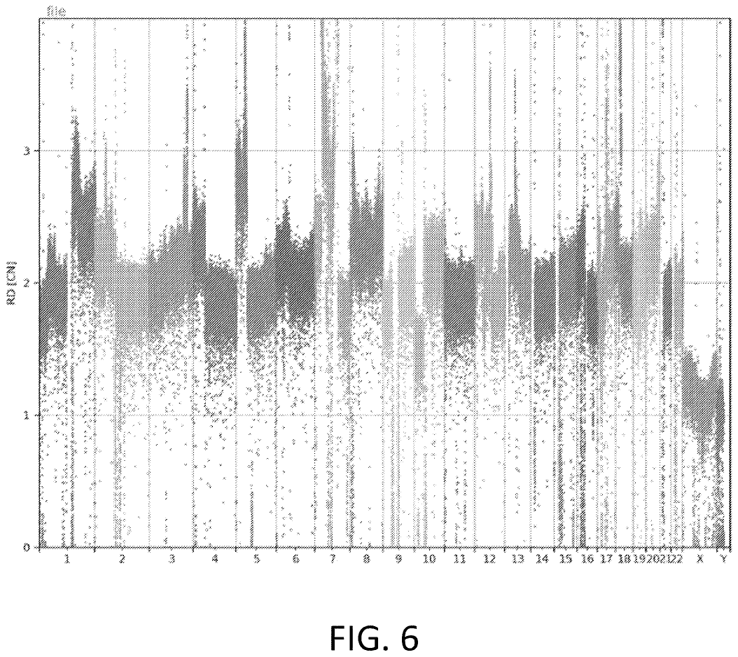

FIG. 2 is a violin plot of the data in FIG. 1, demonstrating the increasing level and frequency of aneuploidy from non-dysplasia to HGD to EAC. The presence of chromosome arm gains or losses was observed in 14 (78% [52-94%]) EAC, 7 (39% [17-64%]) HGD, and 4 (22% [6-48%]) NDBE patients (specificity of 78%). The calls were confirmed by visual analysis of CNVpytor chromosomal scatter plots for every sample. See representative plots in FIG. 3 (AS 0), FIG. 4 (AS 0), FIG. 5 (AS 4), and FIG. 6 (AS 9).

### Example 3

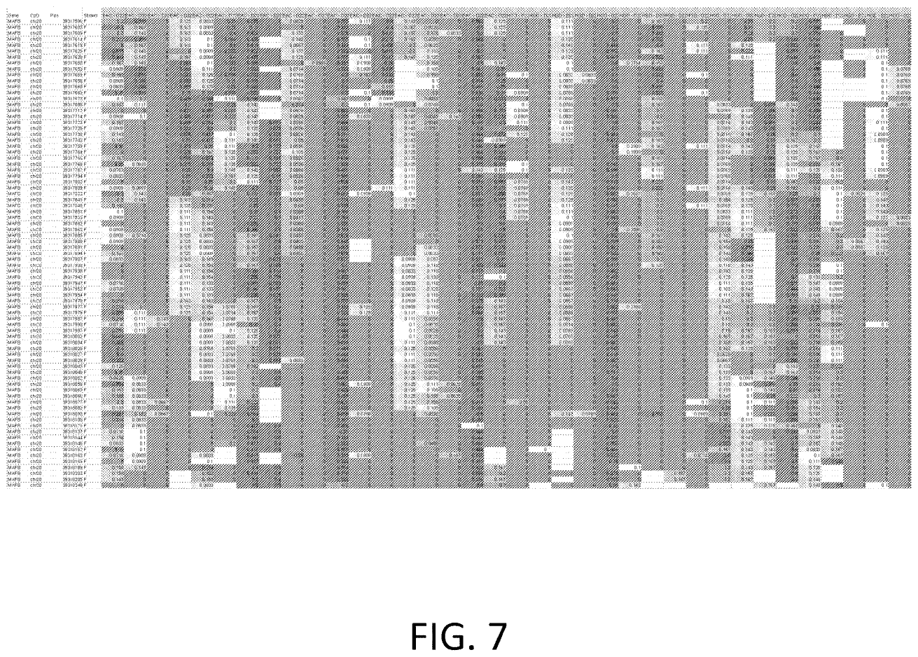

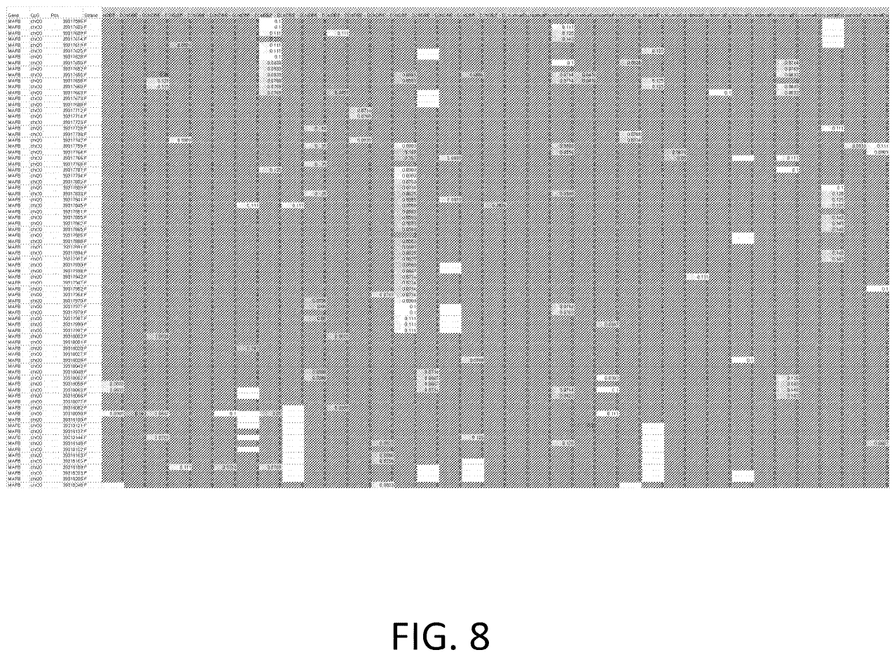

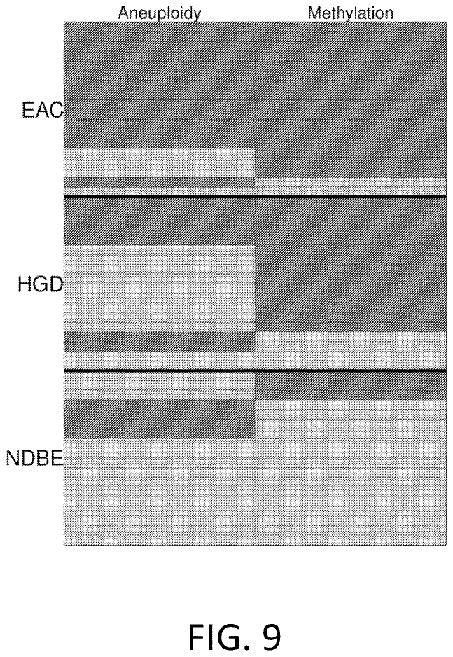

Experiments were also conducted to determine if copy number aberrations (CNAs) and DRMs might complement each other for the detection of BE-associated HGD and EAC from NDBE (and NE) samples. For this example, one DMR was assessed, MAFB (v-maf musculoaponeurotic fibrosarcoma oncogene homolog B), the marker with the highest AUC in the data described above, and detected both in RRBS and WGS studies. The discriminate region is ~1400 bp in length and highly informative (FIGS. 7 and 8). Hypermethylation of MABF was seen in 16 (89% [65-99%]) EAC, 14 (78% [52-94%]) HGD, and 3 (17% [4-42%]) NDBE brushing patients (specificity of 83%). Combining methylation and aneuploidy, 17 (94% [73-100%]) EAC and 16 (89% [65-99%]) HGD were correctly classified, at a fixed specificity of 85% [58-96%] in NDBE samples. One EAC and two HGDs were missed by both marker classes (Table 4 and FIG. 9).

TABLE 4
MAFB methylation complements CNV results. Samples that were negative (i.e., “neg”
in CNV Call column) were those that were missed by CNV, but were identified by
assessing methylation status (i.e., “pos” in Methylation Call column).

| | MABF Avg Fractional | Methylation | CNV | Signal | |
|---|---|---|---|---|---|
| Sample | Methylation | Call | Call | Type | Signal |
| EAC - D22000449 | 0.780118367 | pos | pos | arm | 13 |
| EAC - D22000585 | 0.404614063 | pos | pos | arm | 7 |
| EAC - D22000467 | 0.403146324 | pos | pos | arm | 5 |
| EAC - D22000586 | 0.291672015 | pos | pos | arm | 5 |
| EAC - D22000447 | 0.284567073 | pos | pos | arm | 4 |
| EAC - D22000432 | 0.186296016 | pos | neg | arm | 2 |
| EAC - D22000434 | 0.144443874 | pos | pos | arm | 9 |
| EAC - D22000420 | 0.124481273 | pos | pos | arm | 9 |
| EAC - D22000445 | 0.114021941 | pos | neg | arm | 0 |
| EAC - D22000476 | 0.077308779 | pos | neg | arm | 0 |
| EAC - D22000428 | 0.07101573 | pos | neg | arm | 2 |
| EAC - D22000472 | 0.054672727 | pos | pos | arm | 15 |
| EAC - D22000463 | 0.032661957 | pos | neg | arm | 0 |
| EAC - D22000505 | 0.021073091 | pos | pos | arm | 3 |
| EAC - D22000469 | 0.013040239 | pos | pos | arm | 3 |
| EAC - D22000581 | 0.012033459 | pos | neg | arm | 1 |
| EAC - D22000465 | 0.006739695 | neg | neg | arm | 0 |
| EAC - D22000429 | 0.005411355 | neg | pos | arm | 4 |
| HGD - D22000430 | 0.521568797 | pos | pos | arm | 7 |
| HGD - D22000477 | 0.253069547 | pos | neg | arm | 0 |
| HGD - D22000466 | 0.17209537 | pos | neg | arm | 0 |
| HGD - D22000438 | 0.160744322 | pos | pos | arm | 4 |
| HGD - D22000485 | 0.152476096 | pos | neg | arm | 2 |
| HGD - D22000500 | 0.132259746 | pos | neg | arm | 0 |
| HGD - D22000423 | 0.108790756 | pos | neg | arm | 0 |
| HGD - D22000460 | 0.09278963 | pos | neg | arm | 0 |
| HGD - D22000462 | 0.080694424 | pos | neg | arm | 0 |
| HGD - D22000587 | 0.040969373 | pos | neg | arm | 0 |
| HGD - D22000421 | 0.017221642 | pos | neg | arm | 0 |
| HGD - D22000441 | 0.015931136 | pos | neg | arm | 0 |
| HGD - D22000588 | 0.014146992 | pos | pos | arm | 3 |
| HGD - D22000584 | 0.011062109 | pos | neg | arm | 1 |
| HGD - D22000417 | 0.00894529 | neg | neg | arm | 0 |
| HGD - D22000436 | 0.00557971 | neg | neg | arm | 2 |
| HGD - D22000427 | 0.004768116 | neg | pos | arm | 4 |
| HGD - D22000439 | 0.002964444 | neg | neg | arm | 0 |
| NDBE - D22000502 | 0.016109542 | pos | neg | arm | () |
| NDBE - D22000475 | 0.009644727 | neg | neg | arm | 0 |
| NDBE - D22000458 | 0.009335531 | neg | neg | arm | 0 |
| NDBE - D22000450 | 0.00841845 | neg | neg | arm | 0 |
| NDBE - D22000504 | 0.005989313 | neg | pos | arm | 2 |
| NDBE - D22000580 | 0.005332319 | neg | neg | arm | 0 |
| NDBE - D22000448 | 0.004393015 | neg | neg | arm | 0 |
| NDBE - D22000461 | 0.003903333 | neg | pos | arm | 1 |
| NDBE - D22000456 | 0.003828464 | neg | pos | arm | 2 |
| NDBE - D22000424 | 0.003577068 | neg | neg | arm | 0 |
| NDBE - D22000499 | 0.002989416 | neg | neg | arm | 0 |
| NDBE - D22000583 | 0.002960595 | neg | neg | arm | 0 |
| NDBE - D22000482 | 0.00290942 | neg | neg | arm | 0 |
| NDBE - D22000455 | 0.002838235 | neg | neg | arm | 0 |
| NDBE - D22000503 | 0.001999213 | neg | pos | arm | 2 |
| NDBE - D22000440 | 0.001688806 | neg | neg | arm | 0 |
| NDBE - D22000484 | 0.00150438 | neg | neg | arm | 0 |
| NDBE - D22000454 | 0 | neg | neg | arm | 0 |
| NormalEso - D22000452 | 0.007873962 | neg | neg | arm | 0 |
| NormalEso - D22000443 | 0.007815636 | neg | neg | arm | 0 |
| NormalEso - D22000493 | 0.006165428 | neg | neg | arm | 0 |
| NormalEso - D22000490 | 0.005940741 | neg | neg | arm | 0 |
| NormalEso - D22000473 | 0.004667033 | neg | neg | arm | 0) |
| NormalEso - D22000464 | 0.003137453 | ncg | neg | arm | 0 |
| NormalEso - D22000501 | 0.002865091 | neg | neg | arm | 0 |
| NormalEso - D22000474 | 0.002689641 | neg | neg | arm | 0 |
| NormalEso - D22000451 | 0.002602273 | neg | neg | arm | 0) |
| NormalEso - D22000433 | 0.001721852 | neg | neg | arm | 0 |
| NormalEso - D22000468 | 0.001549587 | neg | neg | arm | 0 |
| NormalEso - D22000489 | 0.00081749 | neg | neg | arm | 0 |
| NormalEso - D22000486 | 0.000720377 | neg | neg | arm | 0 |
| NormalEso - D22000426 | 0.000655797 | neg | neg | arm | 0 |
| NormalEso - D22000491 | 0.000625468 | neg | neg | arm | 0 |
| NormalEso - D22000478 | 0.000559701 | neg | neg | arm | 0 |
| NormalEso - D22000496 | 0.000302909 | neg | neg | arm | 0 |
| NormalEso - D22000452 | 0.007873962 | neg | neg | arm | 0 |

If an additional DMR (e.g., LONRF2) is added to the panel at 100% specificity, one of the missed HGD patients is correctly called without a false positivity hit. Additionally, subsequent estimated cell fraction analysis was performed (using TGCA epigenetic profiles of different cell types) on all the WGS samples (data not shown), and it was found that for the two remaining miscalled cases no columnar epithelial signal was evident, while the squamous signal in both was quite strong. These two brushings may have been either improperly sampled or a mix-up occurred in the classification. The other cases and controls in the WGS study had the appropriate epigenetic profiles.

Taken together, CNV and DMR analysis complement the classification of BE-HGD/EAC from NDBE. Both CNV and DMR analysis can be assayed from endoscopic brush specimens and can augment endoscopic surveillance. For example, aneuploidy develops in the later phase of tumorigenesis; DNA methylation is a very early event, which is why even NDBE samples are generally (but not always) heavily hypermethylated, and is also why the numbers of dysplastic specific DMRs are less than the DMR numbers typically observed with cancer/normal cohorts. Combining these genomic and epigenetic alterations is an advantageous strategy for the detection of dysplasia.

### Example 4

Validation of markers identified in Example 1 was performed by applying the 199 identified DMRs from Table 1 to an independent set of 169 endoscopic esophageal brushing samples. Whole methylome sequencing (shallow) was also performed on these samples to confirm the prior aneuploidy results. Based on a cross-validated DMR selection, a model of four DMRs, including KL, PGBD5, ROR2, and LMX1B, achieved a cross-validated AUC of 0.87 (0.81-0.94, 95% CI) for the identification of HGD/EAC. Incorporating the tMAD score for CNA achieved a cross-validated AUC of 0.92 (0.87-0.98) for the identification of HGD/EAC. At a specificity threshold of 80%, the 4-DMR model identified 35 (85% [71-94%]) EAC, 23 (88% [70-98%]) HGD, 14 (63% [41-83%]) LGD, and 8 (19% [9-34%]) NDBE patients (observed specificity of 81%). The 4-DMR+tMAD model identified 38 (93% [80-98%]) EAC, 23 (88% [70-98%]) HGD, 11 (50% [28-72%]) LGD, and 8 (19% [9-34%]) NDBE patients (observed specificity of 81%).

In this validation experiment, CNA and methylated DNA markers (individually and in combination) showed promising discrimination for the differentiation of BE-HGD/EAC from NDBE. Since both data types can be assayed from endoscopic brush specimens, they could be utilized for the molecular augmentation of histologic analysis in endoscopic surveillance.

### 6. MATERIALS AND METHODS

Marker Discovery. The following materials and methods were used to identify the various DNA methylation markers capable of distinguishing NDBE, HGD, and EAC in a biological sample from a subject. Briefly, DNA was extracted using prospectively collected esophageal brushing samples from patients with NDBE (18), HGD (18), and EAC (18), along with 17 non-BE squamous epithelia samples (NE). High-capacity endoscopic brushings were obtained from the distal 5 cm of the squamous epithelium and cardia in NE patients and from the visible BE mucosa in those with BE. Clinical, endoscopic, and histological data were abstracted and histology was validated by an expert gastrointestinal pathologist prior to execution of the experiment. Enzyme converted methyl-seq (New England Biolabs) libraries were prepared from extracted DNA and sequenced on the Illumina NovaSeq 6000 system. Differentially methylated regions (DMRs) were identified from CpGs with 5× or greater read coverage. Positivity of a DMR was set at a specificity of 85% in the NDBE group. CNAs were called by CNVkit using NE group as reference and aneuploidy events were determined by AneuploidyScore using their default settings. Combinations of methylation and aneuploid status were evaluated using logistic regression.

Patient Samples. Tissues samples were obtained from Mayo Clinic biospecimen repositories with institutional IRB oversight. Endoscopic esophageal brushing samples were collected under Mayo RB protocol 15-004540. The two sample types were not patient-matched and were from completely independent individuals. Samples included esophageal biopsies (FFPE) from patients with and without Barrett's esophagus/EAC; esophageal brushings from patients with and without Barrett's esophagus/EAC; and normal gastric cardia biopsies (FFPE).

Samples were chosen with strict adherence to subject research authorization and inclusion/exclusion criteria. Exclusion criteria included: patient is <18 years old; patient has received chemotherapy class drugs to treat primary esophageal cancer prior to tissue collection; patient has received therapeutic radiation or ablation (photodynamic therapy, radiofrequency or cryotherapy) for the primary esophageal cancer or LGD or HGD or squamous dysplasia/carcinoma prior to sample collection (Note: EMR or ESD alone are not exclusions); patient has history of higher grade disease (i.e., Low grade/High grade/Adenocarcinoma) than the target pathology; and patient has history of pancreatic, gastric, liver (HCC or cholangiocarcinoma), ampullary, or duodenal cancer in last 5 years prior to sample collection.

Exclusion criteria for normal squamous and normal cardia included: patient has a history of esophageal squamous dysplasia or carcinoma or history of BE prior to sample collection; patient has a history of eosinophilic esophagitis prior to sample collection; patient has erosive esophagitis at the time of sample collection; patient has a history of gastric intestinal metaplasia or dysplasia prior to sample collection; patient has had ablation (photodynamic therapy, radiofrequency or cryotherapy) for esophageal or gastric disease.

Exclusion criteria for Non-Dysplastic BE (NDBE) included: patient has documented dysplasia (before or after the date of sample used for this project) sampled from Barrett's Esophagus; patient has had surgical treatment/biopsy/ablation (photodynamic therapy, radiofrequency or cryotherapy) to treat (not just sample but to eradicate) the neoplastic lesions in the esophagus (Note: EMR or ESD are not exclusions).

Inclusion criteria for normal squamous and normal cardia included: patient has normal histology in squamous and/or cardia tissue (biopsies or other sample types).

Inclusion criteria for Non-Dysplastic BE (NDBE) included: patient has histology of Barrett's without dysplasia; patient has one visit before target visit and one visit after target visit with no evidence of dysplasia.

Inclusion criteria for LEG, HGD, and EAC included: patient has histology of Barrett's with dysplasia or esophageal adenocarcinoma.

Brushings consisted of 18 esophageal adenocarcinomas (EAC), 18 Barrett's with high grade dysplasia (HGD), 18 non-dysplastic Barrett's esophagus (NDBE), and 17 normal esophageal squamous epithelia (NE). The latter were sampled from the distal 5 cm of the esophagus and included some cardia. Clinical, endoscopic, and histological data were abstracted, and the latter validated by an independent pathologist.

TABLE 5
Patient Characteristics.

| | NE | NDBE | HGD | EAC |
|---|---|---|---|---|
| | (N = 17) | (N = 18) | (N = 18) | (N = 18) |
| Mean (SD) age | 61 (12) | 63 (8) | 66 (8) | 64 (10) |
| Male sex, N (%) | 10 (59) | 11 (61) | 16 (89) | 15 (83) |
| Mean (SD) BMI | 25.4 (4.6) | 34.1 (6.9) | 29.2 (6.7) | 30.3 (6.3) |
| Ever smoker, N (%) | 8 (47) | 9 (50) | 14 (78) | 13 (28) |
| Mean (SD) maximal | — | 5 (2.7) | 6 (3.2) | 5.5 (4.5) |
| BE segment length | | | | |

Tissues consisted of 3 EACs, 3 gastroesophageal junction cancers (GEJC), 18 HGDs, 13 Barrett's with low grade dysplasia (LGD), 16 NDBEs, 10 NEs, and 17 cystic gastric cardia samples (GC). Tissues were macro-dissected and histology reviewed by an expert gastrointestinal pathologist. Samples were age matched, randomized, and blinded. DNA from tissues were purified using the Qiagen QIAmp FFPE tissue kit (Qiagen, Germantown MD). DNA was re-purified with AMPure XP beads (Beckman-Coulter, Brea CA) and quantified by PicoGreen (Thermo-Fisher, Waltham MA). DNA integrity was assessed using qPCR. Brushing samples were collected and stored in 1 mL of Qiagen cell lysis solution at stored at −80° C. Subsequently, DNA was extracted with the Qiagen Puregene kit modified for a 1 ml sample volume.

Sequencing. Reduced representation bisulfite sequencing (RRBS) sequencing libraries from tissue genomic DNA were prepared using the NuGEN Ovation RRBS Methyl-Seq kit with modifications (Tecan Genomics, Redwood City CA). Samples were combined in a 4-plex format and sequenced by the Mayo Genomics Facility on the Illumina HiSeq 4000 instrument (Illumina, San Diego CA). Reads were processed by Illumina pipeline modules for image analysis and base calling. Secondary analysis was performed using SAAP-RRBS, a Mayo developed bioinformatics suite. Briefly, reads were cleaned-up using Trim-Galore and aligned to the GRCh37/hg19 reference genome build with BSMAP. Methylation ratios were determined by calculating C/(C+T) or conversely, G/(G+A) for reads mapping to reverse strand, for CpGs with coverage≥10× and base quality score≥20.

For the endoscopic brushing samples, 120 ng of DNA was sheared with a Covaris LE220 sonicator (Covaris, Woburn MA) to ~300 bp. Samples were concentrated to 50 uL and whole genome sequencing (WGS) libraries prepared using the NEBNext Enzymatic Methyl-seq kit (New England Biolabs, Ipswich MA). Briefly, the sheared samples underwent end repair, A-tailing, and indexed adaptor ligation. Libraries were then subjected to an enzymatic conversion step, amplified, and discrimination between methylated and unmethylated cytosines. Converted libraries were amplified, quantified, and pooled to a normalized input in a 24-plex format. A total of 3 pools were prepared and sent to the Mayo Genome Analysis Core for sequencing on the Illumina NovaSeq system (Illumina, San Diego CA) using a S4 flow cell (PE 150 cycles). Primary and secondary analysis was performed as above.

Biomarker Selection. RRBS and WGS data were analyzed separately, but with similar metrics. CpGs for inclusion had a depth of coverage of 5 reads (or more) and a variance across subgroups>0. CpGs were then ranked by hypermethylation ratio, namely the number of methylated cytosines at a given locus over the total cytosine count at that site. For NE and GC controls, the ratios were required to be ≤0.01 (1%) and ≤0.05 (5%) for NDBE samples. CpGs which did not meet these criteria were discarded from the results. Subsequently, candidate CpGs were binned by genomic location into DMRs (differentially methylated regions) ranging from approximately 40-2200 bp with a minimum cut-off of 5 CpGs/200 bp region. AUCs for these regions had to be >0.60 and methylation fold-change ratios (FC) between cases and NDBE controls>3 (>5 for the RRBS data; tissues have higher cellular purity). For each candidate region, a 2-D matrix was created which compared individual CpGs in a sample-to-sample fashion for both cases and controls. Final selections required cases to exhibit stretches of coordinated and contiguous hypermethylation of individual CpGs across a DMR sequence on a per sample level. Conversely, for control samples the methylated CpG patterns had to be highly stochastic. DMRs for which one or more NDBE samples have a CpG average methylation value greater than the average methylation of all EACs and HGDs together for that DMR were discarded.

Following regression, DMRs were ranked area under the receiver operating characteristic curve (AUC) and fold-change difference between cases and NDBE controls. No adjustments for false discovery were made during this phase as independent validation was planned apriori.

Biomarker Merging/Validation. A subset of the DMRs was chosen for further development. These were identified by merging the filtered DMRs from two independent patient and sample-type discovery datasets, which had been generated and analyzed separately. This was not simply done at the gene annotation level but at specific genomic locations within the gene. A qualified region was, therefore, one whose genomic coordinates from the two discoveries overlapped or whose endpoints were no more than 500 bp apart.

Copy Number Variation. Copy number variation events in the WGS next generation sequencing (NGS) brushing data were called by CNVkit (github.com/etal/cnvkit) using the NE group as reference and aneuploidy events determined by AneuploidyScore using default settings (github.com/quevedor2/aneuploidy_score). Scatterplots were generated with CNVpytor software (github.com/abyzovlab/CNVpytor) with each sample as its own reference, using a genomic bin size of 10K.

Statistics. RRBS and WGS results were analyzed logistically for individual DMR/MDM performance, for CNV based on chromosomal ARM gain/loss log 2 score, and for combinations of methylation and aneuploidy status.

Validation. For the validation experiment, DNA was extracted from prospectively collected esophageal brushing samples from 169 patients who had NDBE (42), LGD (23), HGD (26), and EAC (41), along with 37 non-BE squamous epithelia samples (NE). Endoscopic brushings were obtained from distal 5 cm of the squamous epithelium and cardia in NE patients and from the visible BE mucosa in those with BE, with one brush used for each 5 cm. Clinical and endoscopic data were abstracted, and histology was confirmed by a gastrointestinal pathologist.

The same sequencing library preparation was used for both methylation and aneuploidy assays, however for the methylation assay, a target capture step was performed prior to sequencing. For the target capture step, custom DNA capture probes were prepared by IDT for the 199 DMRs in Table 1. Targeted (DMR) and shallow whole genome (CNA) sequencing on enzyme converted DNA libraries was performed using the Illumina NextSeq platform. The number of DMRs was reduced by removing those with low coverage and those with high variability in the control group (NDBE). Further variable selection was performed using the VSURF package in R and the final model was generated using random forests comparing NDBE vs HGD/EAC. CNA burden was estimated with the trimmed median absolute deviation (tMAD) score from the ichorCNA package.

TABLE 6
Patient characteristics for validation experiment. One LGD sample was excluded
due to poor sequencing. NE: normal esophagus, NDBE: non-dysplastic BE, LGD: Low
grade dysplasia, HGD: High grade dysplasia, EAC: esophageal adenocarcinoma

| | NE | NDBE | LGD | HGD | EAC |
|---|---|---|---|---|---|
| | (N = 37) | (N = 42) | (N = 23) | (N = 26) | (N = 41) |
| Mean (SD) Age | 47(13) | 62(11) | 69(10) | 67(8) | 68(11) |
| Male Sex, N (%) | 23(62%) | 28(67%) | 17(74%) | 23(88%) | 35(85%) |
| Mean (SD) BMI | 27(7) | 29(6) | 31(7) | 30(5) | 32(6) |
| Ever Smoker, N (%) | 16(43%) | 18(43%) | 17(74%) | 19(73%) | 29(71%) |
| Mean (SD) of BE | — | 4(4) | 5(3) | 4(3) | 5(3) |
| segment length (cm) | | | | | |

Sequences. The various nucleotide sequences referenced in the present disclosure are provided below (see, e.g., Human February 2009 (GRCh37/hg19) Assembly).

MAX.chr15.4912 (DMR 110):
(SEQ ID NO: 1)
CGTGGTTGCTGCGTGTGTCCCGCAGCCCCCTGCAGCCAGCGTCTCCTCGGCCCCGGCCCGACCCTGCCGT
CCCTGCTCTGCTCCCTCAGCGACCGCAGACCCCTCACGCACATGCCCAGCCCTGCAGTCCTACTCTGCCA
CCCCAAGGGGCTTGGGCTTCAGTGGGGGCAGCGTGCGGGGCGTGGAGAGGGAGACGTGTAAAGCCCTGAG
AGGCTCTGCAGCCACAGTCCCGCAGTCAGCGTTCCCTCTCGGCCAAGCCTCCTGGCTGCACATCGTTTCC
CGGGCATCTCAGTGAGACCGGCCTCGGCTCACAGCGGCACGTTGTTTTTACACTTCGGGGACTCTGGTGG
GACCGCGGGAAGCCAGGAGGCGGGCGCGCCCCGGGAGCCGATAGGAAAGTGCAACAGCGCCATCTAGTGG
CCGCGCGGGGAGGCTGCGGAGCGCGCGCCGCGACCCGGGATCCACCGTCCGCAGGAAAACGTGTTCCCTC
CGATGCGTGGTCACAGCGCGGCAGCGCGGCCTTGGGAGCTTCTTAAACAACAGGTTAGCTATCGCTGCCG
CCTCAGTAAGAGAAGTCAAGGCCAAAACGCTTAAAGGATTTTGACCTATTCGGCTTGGTGGAGAGCGCTA
AAAGGGGACGCTATAGCGGGATTCTCAGCTCTCCTGGGCTAGTGGGACCCCCGGTGCGCCGCCGCCGATG
GGGTCTTAGGGCCTGTCTGTCTGGGCTAGAGCCGGCCCGGGAGCCTGTTTGCGGGGAGTGCG.
MAX.chr17.8070 (DMR 115):
(SEQ ID NO: 2)
ATCTCCGATACTCCTCTCCTCAGCTTCCCGGGGGCAGTATGTCCCTTTGGGTCATGGCCTGTGGAGTGGC
CATGACCCAAAGTTCAATTAAGGCTGGCGGGTCCCCAGAGGGGCCTGCTTCCATTCCCCCTTCCCCGCAG
CCCTGCAGGCGTCAGCATGGGGCGAGGTTACCAGTTCCTGACCCAGCAGCTCGTCCTCAGGTGGTCCTGC
TGCTCGGGAGGTCAGCTGCGTGGGGAGCCTGTCCACCTGGCTGATGGGGGTGATGGCCGCGTGGCTGGGA
TTCCTCCTCCAGCCCTGCCCTTCACCGCCATACAGTCTGGGCAGCCCCTTCCTGTCCCGTCCTCCTGCCT
CGCCCTCTGCTGGGGTCTACGGGGCCCAAGTCTTGCTGGAACACAGGCAGGACAAGGGTTCTGGAGGTTC
AGGCCGGAAGTGAAATAAAAGTCGAGAGGGGCCGGGCCCGGCACCCACCGCATCCTGCCTCCGGGCCTCC
TCGGGCCCCTCACACGGTGGGGCAGCCCCTCCTCCAGTGGAGCGGAACGGTGGGCCCCGCTGCCCCTCCA
CCCCTGCGTGTGACACGGACGTGCTTGGAAGCTGGGTTCGTCCTTTCAGTTTTGTTTCCTGGTTGGAAAC
CCCTGAGACTCCTCCCCCTCCCCCCGCCCCTGCCCACCTGTGCTTTCCCAGGCTGGAAGGGGCAGCCCCT
CTCAGCACGTGGGTGGTTAGGCTGTGACGTCCCCAGGCCTCCTGGGCCTGCACCGGGGTCCACCCAGGTG
TGGGCCATGCCCAGGTGCGGG.
MAX.chr19.3439 (DMR 117):
(SEQ ID NO: 3)
CGGTTTCGCTCCCTATCGGGGCCAGGGACGCCTCAGGCTGTCTCGGTCAGGACCTACAGCTCCGGTTGTT
GTCCCAGGCTCTTCCGCGAGGTGCTCTCCTGTCTCCTGACCACCCCCATTCCTCCCCACTCCAGCTCCTC
AGCGAGCGGCTGCGAAGGACGCGCACAACAACACTCCGCGCAGCGGGAAGGTACCGAACTCGCATCGCAG
CCTGAAACTGCTCAACTAAGCTCCCGCCCCGCTGCTCTCTGGTCAATCTAAAGCGAAGACGAGCCTTAGG
GCCAATCAGAAGCGACAGCGGTGGAGTCATGCCCGCCTGCTTGAGGCGCCCTGGCGTCTCATTGGCTATG
CTTGAGAACGAATCCCAGGCTAAGCCACTTAGAAAGGAGCGGAGCCAGCCAATCAGCGGTGCAACCGCCG
CGGGGGCCGGGCCAGAAGCCCCGCAGACAAGCACCTCGGGAGACTGGCGAGGGGCGAGCTCGCAGCTTGT
TAGCCCCGAAGCCCTGCCCAGGGGGAAACCCTGCTGGAGGGAGTCTAACCCCCGGGCCAGTTAATGTTGG
GGCAGCGCAATCGGCCGTTCCCTCTGCGGTATGGTTGGAGGTGGGAGTGCTGACACGTCCGCGCG.
MAX.chr4.4552 (DMR 124):
(SEQ ID NO: 4)
GCCTGGGGCCACTGCTCCTGGGTCCTCAGGACTGCCTGGGGGAAGGTAGTGCATTGTGCAGCGCGCGGTC
CAGAAGTGAAAAGGGAGGCGCGGAGATAAGCTGCCGGCGGAAGTTCCCTCTCCTGCCTGGGCCGACCCGG
CGCTTTACTGCTTCTCACGAAGGTGCGCCGGCTGCTCCAGAAATCGCAGACTGCCTCCAGGAAGAACTTG
CTGGAGTCACAGCAGCTTCTCAGCGACTTGACAGCAGTGATTCAGACTTCAACTTGGGCGGAGGGGCGGG
GGAGGAGAAAGAGATTTCCAGAGAAAACGACTGAGCGGTAGGGAGGGGAAGAGAGACCGAGCCACGCGCC
TCGAGAAGCAGTGCAGAGAGCGGAAGAGACAGAGGCTGCGAGACCTACCCACAGAGACCAAGAGAGACGC
TCGGAGGGGAGACCGCCTTAGGCGCAGAGATTCAGAGGCAGACAGACAGGCAGACAGAAGGATACAGGAA
AGGAATGTCGCCGAAAGGCAGGGACAAACCTGAGTCCCAGAAAAATAAGAGACAACTCCCACACACCAGG
CTGTCCGCGGGCCGCCTTGTCACAGAAAGGCAGCTCCCCAGCCCCGCAGAGTCCCGACAGCTGCCCCCGC
GAAGGTGGGGCGAGGGGGGCTTTTCCG.
MAX.chr6.6743 (DMR 126):
(SEQ ID NO: 5)
CGGACCAAATATAACCATTTCTAAAAGCATTCCTCAGCGCTTGGAACTTGCGAGCTGATGGCTTTTGGTG
GAGCCCAGGAGGGGCGCTGTGGGGCACAGACGTGTGCGGGGCCGGCAGCTGCGGAGAGAGCAGCGCACCC
CAGCCTTCAATTCCCCGCCGAAGATGGGACTCTGCCCAGAGCCGAGGCCCCTGGCTGCTGCCCCGCTCCC
TTGCGGCCGCAGTGACACTCAGAGCCAGATTCCTCAGACAATCTTCACTCCTCCGTGGTGGCTGCCTTGG
GAGGCGTTGCGGGGTGAAGGAAGTCGCAGCTGGTCCCAAGCAGTGGGGAGGGCAGGACAGGGAGTAAGAC
ACCGCCTTCCTGGGGTGCACACGGAGGAGGGGCGCTGCCCAGGAGCGGAGGGGAGGGGAGCGGAGCGCGG
AGAGGAGACCGGTGCCAGACTTCACCCGCCTAGCTCTCGGAACCCCCGGGGGCCGCGCTCGGCTCCGCCT
GGGGGCTACCGGTTCTGGGCCAGGAGAAGAAGTGGCATCCGCGGGGCAGGGCGGGCGGTGACGGCCGGCT
CAGGTGGCGGCCCCACCCCAGCGCCCTTCAGCAGGCCTGGGTCGGTCGGCCTCGGACCCCGTAGGCGCCG
GCGGAGCTGGCGGCAGGGACCCGACAGCTCTGCCGCGGACTCCGCAGGGGACATCCCCCACCCTGGCGGC
CCACCCAGGCGGAGCGCGGCGGGGACCGTGCATCTAGCGCGCCCTGGTGACCTGCGGGCCGAGGCGGGCT
GTGGGGCTTGGCCCCGCGACTCAGCCTTCCGGGCCCACAGCGGCCCTGACCCGGGAAGTGCGGAGGTTTG
GCCGCCTTCAACAGCACCTGCCAGGCCACTGGGAGCAGCGGGGGAGAAGTGGGCTCGCACACCGACGGCC
TCCCGGGTTCGGCGGGGACGACCCGAGCTAGGAGCCCGCGGGGGCCGTGGGAGCTGCTCCGCCGAGGTGG
ACCCGGGGCGCCCGCACCCCTCACCTTCTTTCCGTGGGGTCCGGGGCCAGGGACGCAGCGGGGGACGGCG
GGCATCAGCCGGGCAGCGCCCGAGCCGTCCCTGGCCGGGTCCCCACACCTGCGTGCTCGGGCATCCCCAG
GGTCGGGGGCGCGGTGAATCGTGGTCCCCGCAGCCCGGGGCCCGCCAGGCCCGCGGAGTCAGCCCACCCT
GGCGAGGGGCCCAGGCACCCGCATTTAACCAGTCCCGACCTCGGCACGTGGCGGCGCCGGTGTCGCAGAC
TCGGGCCCACTTCTGGGAGCTTTGGTGCGAGGGGGAAAGTGCCCCTGCGGACGGGCTCGGGTGCCCGGTG
CTGCCCCGCCGCCATCCCCGGCACG.
MAX.chr8.3003 (DMR 129):
(SEQ ID NO: 6)
CGCGCCTCCCGGAACCACGCGTCTCTGTGCACAGACATTCCTGGGGGCAGGCTCCTGTCCTTTAACACAG
TCTCAAAGGAGTCTTCAAAAACAAAAAGTTTACAAGCACTGATAGGGAAAACAGAAGGATGATAACACCA
AGAGGGACTTTCCCGGGAGAGGACGGCTTCCAGGGACTGAGAAAGGATGGGCAAGTGGGCGGGGCCCGGC
GCCGTGCGGGCAGGGCTGGCGCCGGGAGTCCCCAGACTCCCCCGCAGTGGGAAGCACCTCTCCCATTCAC
GCCGGGCAGGACACCTGGCCGGGCGGGGGAGGCAGCGCAAGGGCCGGCCGGGGAGTACGGGACTCGAGCC
GGGGACCTGAGGCAGGAGCCAAGCATCGTCGCAGGGCAACCAGCAGAACGGAGAGGGAGGCGCGGGGGCG
AAGGCTGGCGGGAGCCGCGCTGAGGGCAGGAGCCCGGAGCCCCCTAGGGCAGCGCCGATCCGCCCGCCCC
GTCCCGCCGAGCTGGGCCTCCGTCTGTGGCCTGCGCAGCCAGGGTCGCCAAGCCG.

What is claimed is:

1. A method of characterizing a biological sample, the method comprising:
determining a methylation profile in at least one differentially methylated region (DMR) of a DNA sample obtained from a subject having or suspected of having an esophageal cancer or pre-cancer by treating the sample with a reagent that modifies DNA in a methylation-specific manner.

2. The method of claim 1, wherein the methylation profile in the at least one DMR indicates the subject has or is suspected of having high grade dysplastic Barrett's Esophagus or esophageal adenocarcinoma (EAC).

3. The method of claim 1 or claim 2, wherein the at least one DMR is from a gene selected from ACVRL1, ADAMTS8, ADAP2, ADRBK2, AKR1B1, ANK1, ANKRD13B, ANXA6, ARNT2, B4GALNT2, BACH2, BCL11A, BSCL2, C12orf53, C14orf82, C17orf107, C18orf1, C1orf95, C5orf42, CACNA1C, CAMK1D, CAMTA1, CBX6, CCDC85A, CCKBR, CD38, CDKN2A, CH25H, CHST1, CHST15, CNTLN, CRHR1, CRTC1, CXCR4, CYP1B1, DCTN2, DIDO1, DMKN, DSE, DYNC1I1, EML6, ENOX1, EPHA4, ESRRG, FAM176A, FAM78B, FBXO10, FERMT2, FHOD3, FLJ45079, FMNL1, FOXP2, FRMD4B, FZD8, GALNTL1, GALNTL4, GAS1, GBGT1, GLIPR2, GNAI1, GNAL, GPR37, GRASP, GRID1, GRM8, GSC, HAR1A, HCN2, HEY2, HIST1H2BE, HS3ST3B1, HTR7, IGFBP2, INHA, INSRR, IRX3, ISM2, KCNG3, KCNK4, KCNS2, KCTD15, KIAA1522, KIAA1614, KIF26A, KL, KLF15, KLHL10, KRT77, KSR2, LBH, LMX1A, LMX1B, LOC100526820, LONRF2, LRFN2, LRRN1, MAF, MAFB, MARK1, ADAMTSL4-AS1, PGBD5, HSPA12A, SFTPD, LOC107984507, LOC100128253, CISTR, SLC16A7, CTXND1, MAX.chr15.4912, ZNF423, RBFOX1, LOC105376772, GSE1, MAX.chr17.8070, ZNF709, MAX.chr19.3439, GREB1, CYP27C1, AC068134.6, LOC388780, STOX2, PPARGC1A, MAX.chr4.4552, PDZD2, MAX.chr6.6743, LPAL2, CDK14, MAX.chr8.3003, MCOLN2, MEGF11, MFSD11, MRC2, MSX1, NAT8L, NAV1, NBEA, NCRNA00092, NECAB2, NEURL, NGEF, NHLH2, NKD1, NLGN1, NOG, NPPC, NR3C1, NRXN2, NTN1, OBSCN, OCA2, OSBPL1A, OXR1, P2RY1, PDE2A, PDE9A, PDGFRA, PID1, PLCG2, PLCL1, PNPLA3, POU3F1, POU3F2, PRKACB, PRKAR2B, PRKG1, PRR18, PRR5L, PTHLH, PYGL, RARG, RHBDL3, RIMS2, ROR2, RPRML, SDK2, SOX9, SYNGR1, TAC4, TNFRSF19, TRANK1, TSPAN33, TSPAN4, TSPAN5, UBE2E2, UCHL1, UNC5A, VASH2, VIM, WNT6, ZBTB10, ZNF680, ZNF738, ZNF808, and ZNF845.

4. The method of claim 1 or claim 2, wherein the at least one DMR is from a gene selected from ACVRL1, ADAMTS8, ADAP2, ADRBK2, AKR1B1, ANK1, ANKRD13B, ANXA6, ARNT2, B4GALNT2, BACH2, BCL11A, BSCL2, C14orf82, C18orf1, C1orf95, C5orf42, CAMK1D, CAMTA1, CCDC85A, CD38, CDKN2A, CHST1, CHST15, CRHR1, CYP1B1, DIDO1, DSE, DYNC1I1, EML6, ENOX1, EPHA4, FAM176A, FBXO10, FERMT2, FHOD3, FLJ45079, FMNL1, FRMD4B, FZD8, GALNTL1, GALNTL4, GAS1, GBGT1, GLIPR2, GNAI1, GNAL, GRASP, GRID1, GRM8, GSC, HAR1A, HCN2, HEY2, HIST1H2BE, HS3ST3B1, HTR7, IGFBP2, INHA, IRX3, ISM2, KCNG3, KCNK4, KCNS2, KCTD15, KIAA1614, KL, KLHL10, KSR2, LBH, LMX1A, LMX1B, LOC100526820, LONRF2, LRFN2, MAFB, MARK1, PGBD5, HSPA12A, SFTPD, LOC107984507, LOC100128253, CISTR, SLC16A7, CTXND1, ZNF423, LOC105376772, GSE1, MAX.chr19.3439, GREB1, CYP27C1, AC068134.6, LOC388780, STOX2, PPARGC1A, PDZD2, MAX.chr6.6743, LPAL2, CDK14, MCOLN2, MEGF11, MRC2, MSX1, NAT8L, NAV1, NBEA, NCRNA00092, NEURL, NGEF, NHLH2, NKD1, NLGN1, NOG, NPPC, NR3C1, NRXN2, OBSCN, OCA2, OSBPL1A, OXR1, P2RY1, PDE2A, PDGFRA, PID1, PLCG2, PLCL1, PNPLA3, POU3F1, POU3F2, PRKACB, PRKAR2B, PRKG1, PRR18, PRR5L, PTHLH, PYGL, RHBDL3, RIMS2, ROR2, RPRML, SDK2, SOX9, SYNGR1, TAC4, TRANK1, TSPAN4, UBE2E2, UCHL1, UNC5A, VASH2, ZBTB10, ZNF680, ZNF709, ZNF738, ZNF808, and ZNF845.

5. The method of claim 1 or claim 2, wherein the at least one DMR is from a gene selected from BACH2, C5orf42, FHOD3, HIST1H2BE, IRX3, KIAA1614, LONRF2, MAFB, PDGFRA, PID1, POU3F1, PRR5L, RHBDL3, and SDK2.

6. The method of claim 1 or claim 2, wherein the at least one DMR is from a gene selected from KL, PGBD5, ROR2, and LMX1B.

7. The method of any one of claims 1 to 6, wherein the at least one DMR is associated with an area under a ROC curve (AUC) greater than or equal to 0.5, and wherein the ROC curve discriminates between a subject having or suspected of having high grade dysplastic Barrett's Esophagus or esophageal adenocarcinoma (EAC) and a control DNA sample.

8. The method of any one of claims 1 to 6, wherein the at least one DMR is associated with an area under a ROC curve (AUC) greater than or equal to 0.75, and wherein the ROC curve discriminates between a subject having or suspected of having high grade dysplastic Barrett's Esophagus or esophageal adenocarcinoma (EAC) and a control DNA sample.

9. The method of any one of claims 1 to 6, wherein the at least one DMR is associated with a methylation fold-change (FC) ratio greater than or equal to 3.0, and wherein the FC ratio discriminates between a subject having or suspected of having high grade dysplastic Barrett's Esophagus or esophageal adenocarcinoma (EAC) and a control DNA sample.

10. The method of any one of claims 1 to 6, wherein the at least one DMR is associated with a methylation fold-change (FC) ratio greater than or equal to 5.0, and wherein the FC ratio discriminates between a subject having or suspected of having high grade dysplastic Barrett's Esophagus or esophageal adenocarcinoma (EAC) and a control DNA sample.

11. The method of any one of claims 1 to 10, wherein the method further comprises determining copy number variation (CNV) for the DNA sample from the subject; and wherein the CNV discriminates between a subject having or suspected of having high grade dysplastic Barrett's Esophagus or esophageal adenocarcinoma (EAC) and a control DNA sample.

12. The method of any one of claims 1 to 10, wherein the method further comprises determining an aneuploidy score (AS) for the DNA sample from the subject; and wherein the AS discriminates between a subject having or suspected of having high grade dysplastic Barrett's Esophagus or esophageal adenocarcinoma (EAC) and a control DNA sample.

13. The method of any one of claims 1 to 12, wherein the at least one DMR comprises an increased methylation percentage as compared to a control DNA sample.

14. The method of any one of claims 1 to 12, wherein the at least one DMR comprises an increased hypermethylation ratio as compared to a control DNA sample.

15. The method of any one of claims 1 to 12, wherein the at least one DMR comprises an increased AS as compared to the control DNA sample.

16. The method of any one of claims 7 to 15, wherein the control DNA sample is from a subject that does not have an esophageal cancer or pre-cancer.

17. The method of any one of claims 7 to 15, wherein the control DNA sample is from a subject that has non-dysplastic Barrett's Esophagus (NDBE).

18. The method of any one of claims 7 to 17, wherein the control DNA sample is selected from a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and a stool sample.

19. The method of claim 18, wherein the tissue sample is an esophageal tissue sample or a gastric cardia sample.

20. The method of any one of claims 1 to 19, wherein the biological sample is selected from a tissue sample, a blood sample, a plasma sample, a serum sample, a whole blood sample, a buffy coat sample, a secretion sample, an organ secretion sample, a cerebrospinal fluid (CSF) sample, a saliva sample, a urine sample, and a stool sample.

21. The method of claim 20, wherein the tissue sample is an esophageal tissue sample.

22. The method of claim 20, wherein the tissue sample is an endoscopic esophageal brushing sample.

23. The method of any one of claims 1 to 13, wherein the subject is a human.

24. The method of any one of claims 1 to 13, wherein the biological sample is obtained from the subject, and wherein the method further comprises extracting the DNA sample from the biological sample.

25. The method of any one of claims 1 to 24, wherein the biological sample is collected with a collection device.

26. The method of any one of claims 1 to 25, wherein the reagent that modifies DNA in a methylation-specific manner is a borane reducing agent.

27. The method of any one of claims 1 to 25, wherein the reagent that modifies DNA in a methylation-specific manner comprises one or more of a methylation-sensitive restriction enzyme, a methylation-dependent restriction enzyme, and a bisulfite reagent.

28. The method of any one of claims 1 to 27, wherein determining the methylation profile of at least one DMR comprises amplifying at least a portion of the DMR using a set of primers.

29. The method of any one of claims 1 to 28, wherein determining the methylation profile of at least one DMR comprises performing at least one of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

30. The method of any one of claims 1 to 29, wherein determining the methylation profile of at least one DMR comprises determining the presence or absence of methylation at one or more CpG sites.

31. The method of claim 30, wherein the one or more CpG sites are present in a coding region, a non-coding region, and/or a regulatory region of a gene.

32. The method of any one of claims 1 to 31, wherein determining the methylation profile of at least one DMR comprises determining a methylation frequency.

33. The method of any one of claims 1 to 31, wherein determining the methylation profile of at least one DMR comprises determining a methylation pattern.

34. The method of any one of claims 1 to 33, wherein the methylation profile in the at least one DMR and the CNV and/or the AS are determined using the same DNA sample obtained from the subject.

35. The method of any one of claims 1 to 33, wherein the methylation profile in the at least one DMR and the CNV and/or the AS are determined using a single DNA sample obtained from the subject.

36. The method of claim 34 or claim 35, wherein the sample has been treated with a reagent that modifies DNA in a methylation-specific manner
